SEQUENCE LISTING (51 sequences)

SEQ ID NO: 1 - protein, 145 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 2 - DNA, 438 bp, Glycine max
  source 1..438: mol_type = other DNA
atgggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaa

SEQ ID NO: 3 - protein, 154 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 4 - DNA, 465 bp, Bos taurus
  source 1..465: mol_type = other DNA
atgggcctctctgacggcgagtggcagcttgtccttaacgcctggggcaaggtcgaggctgacgtcgctggtcacggccaggaggtccttatccgtcttttcaccggccaccccgagaccctcgagaagttcgacaagttcaagcacctcaagaccgaggccgagatgaaggcctccgaggacctcaagaagcacggcaacaccgtcctcaccgcccttggcggcatccttaagaagaagggccaccacgaggccgaggtcaagcacctcgccgagtcccacgccaacaagcacaagatccccgtcaagtacctcgagttcatctccgacgccatcatccacgtcctccacgccaagcacccctctgacttcggcgctgacgctcaggctgccatgtctaaggccctcgagctcttccgcaacgacatggccgcccagtacaaggtcctcggcttccacggctaa

SEQ ID NO: 5 - protein, 181 aa, Hordeum vulgare
ADPPPVHDTDGHELRADANYYVLSANRAHGGGLTMAPGHGRHCPLFVSQDPNGQHDGFPVRITPYGVAPSDKIIRLSTDVRISFRAYTTCLQSTEWHIDSELAAGRRHVITGPVKDPSPSGRENAFRIEKYHGAEVSEYKLMSCGDWCQDLGVFRDLKGGAWFLGATEPYHVVVFKKAPPA

SEQ ID NO: 6 - DNA, 549 bp, Hordeum vulgare
  source 1..549: mol_type = other DNA
atggcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgcctaa

SEQ ID NO: 7 - protein, 479 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPT

SEQ ID NO: 8 - DNA, 1571 bp, Trichoderma reesei
  source 1..1571: mol_type = other DNA
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacctaa

SEQ ID NO: 9 - protein, 514 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 10 - DNA, 1676 bp, Trichoderma reesei
  source 1..1676: mol_type = other DNA
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 11 - DNA, 1521 bp, Trichoderma reesei
  source 1..1521: mol_type = other DNA
gagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcg

SEQ ID NO: 12 - DNA, 1460 bp, Trichoderma reesei
  source 1..1460: mol_type = other DNA
ctacaacatcgtcctgcagttcctatatgatacgtataacccattgatacatccaagtatccagaggtgtatggaaatatcagatcaagacctctctcttctaagaaacctagaaccagacgctggtagtataataagcacactgtgactcgcttaggcccttaagcttaggccggcttgcttactattaacctctcataaacgctactgcaatgattggaaacttcttatagtagaatgaggcaataagacgcatctcaggtcacatatagtcttatgtttgaaacccctcactactgccatttatcttgtggaaatatctattatttcagtctatacgtaatgaaggcacttttcaggatctcttccctaagcttgtataagcaggtttgttgccgtaaccattctgtctcctcgcctaatacctgtgaagcacagaatacgtttattctataagagacgtcttaccttccatcgagattgaaagcttaaaccgtctacaacggatgccctcatcatgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacactgaattctaggctaggtatgcgaggcacgcggatctagggcagactgggcattgcatagctatggtgtagtagaactcccgtcaacggctattctcacctagactttccccttcgaactgacaagttgttatattgcctgtgtaccaagcgctaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctctgtgtattg

SEQ ID NO: 13 - DNA, 51 bp, Trichoderma reesei
  source 1..51: mol_type = other DNA
atgtatcggaagttggccgtcatctcggccttcttggccacagctcgtgct

SEQ ID NO: 14 - DNA, 138 bp, Trichoderma reesei
  source 1..138: mol_type = other DNA
atgcagaccttcggtgcttttctcgtttccttcctcgccgccaggtaagtagacactcactggaattcgttcctttcccgatcatcatgaaagcaagtagactgactgaaccaaacaactagcggcctggccgcggcc

SEQ ID NO: 15 - DNA, 375 bp, Trichoderma reesei
  source 1..375: mol_type = other DNA
acccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccag

SEQ ID NO: 16 - DNA, 711 bp, Trichoderma reesei
  source 1..711: mol_type = other DNA
atggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactga

SEQ ID NO: 17 - DNA, 1900 bp, Aspergillus nidulans
  source 1..1900: mol_type = other DNA
atgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatag

SEQ ID NO: 18 - DNA, 21 bp, Saccharomyces cerevisiae
  source 1..21: mol_type = other DNA
aaggtcgccgtcgagaagcga

SEQ ID NO: 19 - protein, 146 aa, Phaseolus vulgaris
MGAFTEKQEALVNSSWEAFKGNIPQYSVVFYTSILEKAPAAKNLFSFLANGVDPTNPKLTAHAESLFGLVRDSAAQLRANGAVVADAALGSIHSQKGVNDSQFLVVKEALLKTLKEAVGDKWTDELSTALELAYDELAAAIKKAYA

SEQ ID NO: 20 - DNA, 441 bp, Phaseolus vulgaris
  source 1..441: mol_type = other DNA
atgggcgccttcactgagaagcaggaggccctggtcaacagcagctgggaggccttcaagggcaacatcccccagtacagcgtcgtcttctacaccagcatcctggagaaggcccctgccgccaagaacctgttcagcttcctggccaacggcgtcgaccctaccaaccctaagctgactgcccacgccgagtctctgttcggcctggtccgcgactctgctgctcagcttagggctaacggcgctgtcgtcgctgatgctgcccttggcagcatccacagccagaagggcgtcaacgacagccagttcctggtcgtcaaggaggccctgctgaagaccctgaaggaggccgtcggcgacaagtggactgacgagctttctactgccctggagctggcctacgacgagctggccgccgccatcaagaaggcctacgcctaa

SEQ ID NO: 21 - DNA, 23 bp, synthetic construct
  source 1..23: mol_type = other DNA
tgtgcgagatccatgactgcgtt

SEQ ID NO: 22 - DNA, 31 bp, synthetic construct
  source 1..31: mol_type = other DNA
atagtatcgtcttgagttattccgagttccg

SEQ ID NO: 23 - DNA, 25 bp, synthetic construct
  source 1..25: mol_type = other DNA
tgatcgcgccagctccgtgcgaaag

SEQ ID NO: 24 - DNA, 27 bp, synthetic construct
  source 1..27: mol_type = other DNA
cctctaaacaagtgtacctgtgcattc

SEQ ID NO: 25 - DNA, 20 bp, synthetic construct
  source 1..20: mol_type = other DNA
cccagattccggccagatag

SEQ ID NO: 26 - DNA, 20 bp, synthetic construct
  source 1..20: mol_type = other DNA
tcgaactgttgtgagcccag

SEQ ID NO: 27 - DNA, 6083 bp, synthetic construct
  source 1..6083: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggacaagcgtaactagctagcatttggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctggcgccttcactgagaagcaggaggccctggtcaacagcagctgggaggccttcaagggcaacatcccccagtacagcgtcgtcttctacaccagcatcctggagaaggcccctgccgccaagaacctgttcagcttcctggccaacggcgtcgaccctaccaaccctaagctgactgcccacgccgagtctctgttcggcctggtccgcgactctgctgctcagcttagggctaacggcgctgtcgtcgctgatgctgcccttggcagcatccacagccagaagggcgtcaacgacagccagttcctggtcgtcaaggaggccctgctgaagaccctgaaggaggccgtcggcgacaagtggactgacgagctttctactgccctggagctggcctacgacgagctggccgccgccatcaagaaggcctacgcctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtacactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaagtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggc

SEQ ID NO: 28 - DNA, 6167 bp, synthetic construct
  source 1..6167: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggacaagcgtaactagctagcatttggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgcagaccttcggtgcttttctcgtttccttcctcgccgccaggtaagtagacactcactggaattcgttcctttcccgatcatcatgaaagcaagtagactgactgaaccaaacaactagcggcctggccgcggccggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtacactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaagtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggc

SEQ ID NO: 29 - DNA, 8059 bp, synthetic construct
  source 1..8059: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctaccaaggtcgccgtcgagaagcgaggcgccttcaccgagaagcaggaggccctcgtcagcagcagcttcgaggccttcaaggccaacatcccccagtacagcgtcgtcttttacaccagcatcctcgagaaggcccctgccgccaaggacctcttcagcttcctcagcaacggcgtcgaccccagcaaccccaagctcaccggccacgccgagaagctgtttggcctcgtccgcgactctgccggccagctcaaggccaacggcaccgtcgtcgccgacgccgccctcggcagcatccacgcccagaaggccattaccgacccccagttcgtcgtcgtcaaggaggccctcctcaagaccatcaaggaggccgtcggcgacaagtggtccgacgagctgtctagcgcctgggaggtcgcctacgacgagctggccgccgccatcaagaaggcctttcatcaccatcaccaccactaataagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggc

SEQ ID NO: 30 - DNA, 8143 bp, synthetic construct
  source 1..8143: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggc

SEQ ID NO: 31 - DNA, 8144 bp, synthetic construct
  source 1..8144: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttccatcaccatcaccaccactaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactat

SEQ ID NO: 32 - DNA, 8153 bp, synthetic construct
  source 1..8153: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcctctctgacggcgagtggcagcttgtccttaacgcctggggcaaggtcgaggctgacgtcgctggtcacggccaggaggtccttatccgtcttttcaccggccaccccgagaccctcgagaagttcgacaagttcaagcacctcaagaccgaggccgagatgaaggcctccgaggacctcaagaagcacggcaacaccgtcctcaccgcccttggcggcatccttaagaagaagggccaccacgaggccgaggtcaagcacctcgccgagtcccacgccaacaagcacaagatccccgtcaagtacctcgagttcatctccgacgccatcatccacgtcctccacgccaagcacccctctgacttcggcgctgacgctcaggctgccatgtctaaggccctcgagctcttccgcaacgacatggccgcccagtacaaggtcctcggcttccacggctaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactat

SEQ ID NO: 33 - DNA, 8171 bp, synthetic construct
  source 1..8171: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcctctctgacggcgagtggcagcttgtccttaacgcctggggcaaggtcgaggctgacgtcgctggtcacggccaggaggtccttatccgtcttttcaccggccaccccgagaccctcgagaagttcgacaagttcaagcacctcaagaccgaggccgagatgaaggcctccgaggacctcaagaagcacggcaacaccgtcctcaccgcccttggcggcatccttaagaagaagggccaccacgaggccgaggtcaagcacctcgccgagtcccacgccaacaagcacaagatccccgtcaagtacctcgagttcatctccgacgccatcatccacgtcctccacgccaagcacccctctgacttcggcgctgacgctcaggctgccatgtctaaggccctcgagctcttccgcaacgacatggccgcccagtacaaggtcctcggcttccacggccatcaccatcaccaccactaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactat

SEQ ID NO: 34 - DNA, 6820 bp, synthetic construct
  source 1..6820: mol_type = other DNA
gggtgctgcttattcgcttgtttggttttggctgaggaggcctccctctctctgacatccgtgtgagcatgagtgtaggtagtatgaggcatttggatttggggccggggcagggctgtgctggccggtttctgtgtgctttgttgcggtgaaggggcctttcgctgtttggggtttaccgtgtctagaccggtcgatgtgctgccattgacgggaacgagcagctggcgctggtgtatgtaagtcgggcaaagtggtgggctggtggactggtggattgaacaagattgttactccatgtctttgctttggcagtgtctgtctatggtcattctatacgtgtaagtgatttgatggacttgattctcatagacaagtctctaagttggctctttgcatgtggtcttactccagggccttatcagtatcgagcaataggtaccctttgatgctacggtgtatacatgtacgtaggtaggttgaatgcgagcattctgtgttgtgtaggcatgtaaggcattgcttcaaacagtgtatgactgctaccacttgctatcctgcatgctgaccgcttgatatgataatacggaacgacatacaaaggacacacaaacagctgcaaaagaggtagaaccagagaacacctgctctgggggcccagccggtggaagcttgcccgctgtttgcagctcttacgccatgcccgtcagcgaacccccccagctcgtacccgcgactttggcgagcccatcagggccagtgccgactcgagggagtccgcactgtctggaccattacgcacgaggctggagcactgtagcgtataattgcatattcttgcatcttcctgcataggtacgcctgcagcagaacccatgacggctcctcgtcccttgggttgagagatgcgggagagggctattctcacctagactttccccttcgaactgacaagttgttatattgcctgtgtaccaagcgctaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctctgtgtattgcaccatgcagaccttcggtgcttttctcgtttccttcctcgccgccaggtaagttggccttgatgaaccatatcatatatcgccgagaagtggaccgcgtgctgagactgagacagcggcctggccgcggccgcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgccggcggcggtagcggcggtggcagcggtggcgtcgccgttgagaagcgcgcccctaccaagaagaagccctggaagtgctgcgacaacatcgagcgcctccccaccaagaccaacccgccccagtggcgctgcaacgacgagctcgagcccagcaagtgcgtcgctcagtgcgaggtctgccaggaggctcccggtcccttccccggaccgctcatctgcagcgacgtctactggggcgctgaccccggccccttctgcactcctcgcccctggggcgactgctgcaccaacaccacctgcacccgctctatccctcccatctgccgctgcaacgacaaggtcaagaagtgcgccgctgcctgcaaggactgcaagcgcgtcaagtccagcaagccgccccgctacgtctgccaggaccagttcaccggccagcctggacccaagtgcaagcactcctgcgagaactaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccaggaatagctcgcccgctggaggcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagaggtaccgcaaggatcggtgatgttgtcgaaccggatcgctgcccacgcctcgcctcatgtggcatggcaagcaatgttgcctccccatactttgccatgagcttatcacaaagcgaggccttggcatctaggaacacgcccacgcccccttgaagcgagctgcctctggcctcctccctctgtctcacctccccgccttcagtctcttggaggattcacgtccgagtatgagcggcactctgaactcttctcagtgtacgccaacataagaaatcacgagtttcttgaagcccggacgcttcgatcataagtcaaaagggtcagtgagagagaggaacttgctactactattcacaggtatccgatctcagggcacaacctcccagccctaaacacctaaaagcgggctcggcagcgaggccatttagtggccgctaactgcgtcccttgatctgggtagtgctgccctcttggcctatcacaggcatcgttgaccagcaagggcgacaaatgcagcaacaggagattgacggagggtcccgagtgaaatgcttggcatcgtggctgtttgtctgcgtgtgttgtgtgtgtgtgtgtgtgtgtttgtgtgtgtgtgaaggcagaaattcataaaagcctatatcaaatacgaagaacggctcgtcgagctgcttctgttgctctggtttcgaattcaccatgggcaagctgtcaagtgtgtttatgtctgttgtagagtcgctacactggagatgatctctggattatacaacagctcaatctcttgcccttctcccctcctctcctcgaaaccgatgggctaacatgaattgccctcagctcgtcatgtcatcatagcaaattcagcagccacgcggccatgccagtcgcgccgacagaccaagacctagtcagcctcgaacacaccctcagctagggcaaccagaaaatgtccctcagcctacggaatactctcagggcataccttacgatagcagcgccaacgccaatgttctagcgctcgaacaacaggcccgttctgggttgcgccacttgtcaaagttttccttctgctgtcctatccgttacaatggaattccgtcttgacgcatagctacctggaccggctgagccagcatctaccaagatgaccacggcaaccagcggcattataagtcatggatattgctcctgtcaacgcttagacggtcgagtgctaagttgattgcggccgcggta

SEQ ID NO: 35 - DNA, 7193 bp, synthetic construct
  source 1..7193: mol_type = other DNA
tgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgcagaccttcggtgcttttctcgtttccttcctcgccgccaggtaagttggccttgatgaaccatatcatatatcgccgagaagtggaccgcgtgctgagactgagacagcggcctggccgcggccgcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgcccctacccagtctcactacggccagtgcggcggtattggctacagcggccccacggtctgcgccagcggcacaacttgccaggtcctgaacccttactactctcagtgcctgaaggtcgccgtcgagaagcgaggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactat

SEQ ID NO: 36 - DNA, 7932 bp, synthetic construct
  source 1..7932: mol_type = other DNA
caagcgtaactagctagcatttggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtacactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaagtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcgga

SEQ ID NO: 37 - DNA, 8019 bp, synthetic construct
  source 1..8019: mol_type = other DNA
caagcgtaactagctagcatttggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgcagaccttcggtgcttttctcgtttccttcctcgccgccaggtaagtagacactcactggaattcgttcctttcccgatcatcatgaaagcaagtagactgactgaaccaaacaactagcggcctggccgcggccggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtacactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaagtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcgga

SEQ ID NO: 38 - DNA, 9911 bp, synthetic construct
  source 1..9911: mol_type = other DNA
cagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctaccaaggtcgccgtcgagaagcgaggcgccttcaccgagaagcaggaggccctcgtcagcagcagcttcgaggccttcaaggccaacatcccccagtacagcgtcgtcttttacaccagcatcctcgagaaggcccctgccgccaaggacctcttcagcttcctcagcaacggcgtcgaccccagcaaccccaagctcaccggccacgccgagaagctgtttggcctcgtccgcgactctgccggccagctcaaggccaacggcaccgtcgtcgccgacgccgccctcggcagcatccacgcccagaaggccattaccgacccccagttcgtcgtcgtcaaggaggccctcctcaagaccatcaaggaggccgtcggcgacaagtggtccgacgagctgtctagcgcctgggaggtcgcctacgacgagctggccgccgccatcaagaaggcctttcatcaccatcaccaccactaataagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

SEQ ID NO: 39 - DNA, 9995 bp, synthetic construct
  source 1..9995: mol_type = other DNA
cagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

SEQ ID NO: 40 - DNA, 10013 bp, synthetic construct
  source 1..10013: mol_type = other DNA
cagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttccatcaccatcaccaccactaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

SEQ ID NO: 41 - DNA, 10022 bp, synthetic construct
  source 1..10022: mol_type = other DNA
cagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcctctctgacggcgagtggcagcttgtccttaacgcctggggcaaggtcgaggctgacgtcgctggtcacggccaggaggtccttatccgtcttttcaccggccaccccgagaccctcgagaagttcgacaagttcaagcacctcaagaccgaggccgagatgaaggcctccgaggacctcaagaagcacggcaacaccgtcctcaccgcccttggcggcatccttaagaagaagggccaccacgaggccgaggtcaagcacctcgccgagtcccacgccaacaagcacaagatccccgtcaagtacctcgagttcatctccgacgccatcatccacgtcctccacgccaagcacccctctgacttcggcgctgacgctcaggctgccatgtctaaggccctcgagctcttccgcaacgacatggccgcccagtacaaggtcctcggcttccacggctaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

SEQ ID NO: 42 - DNA, 10040 bp, synthetic construct
  source 1..10040: mol_type = other DNA
cagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgaaacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgctatcttcttgttggctcccagctgactggccaattcaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgaaacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgaaacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgaaggtcgccgtcgagaagcgaggcctctctgacggcgagtggcagcttgtccttaacgcctggggcaaggtcgaggctgacgtcgctggtcacggccaggaggtccttatccgtcttttcaccggccaccccgagaccctcgagaagttcgacaagttcaagcacctcaagaccgaggccgagatgaaggcctccgaggacctcaagaagcacggcaacaccgtcctcaccgcccttggcggcatccttaagaagaagggccaccacgaggccgaggtcaagcacctcgccgagtcccacgccaacaagcacaagatccccgtcaagtacctcgagttcatctccgacgccatcatccacgtcctccacgccaagcacccctctgacttcggcgctgacgctcaggctgccatgtctaaggccctcgagctcttccgcaacgacatggccgcccagtacaaggtcctcggcttccacggccatcaccatcaccaccactaagcttacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

SEQ ID NO: 43 - DNA, 8795 bp, synthetic construct
  source 1..8795: mol_type = other DNA
cagcaggcgtatttagcggacaagcgtaactagctagcatttggatccgtgggactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccggtacccaccatgtaagcttctacaacatcgtcctgcagttcctatatgatacgtataacccattgatacatccaagtatccagaggtgtatggaaatatcagatcaagacctctctcttctaagaaacctagaaccagacgctggtagtataataagcacactgtgactcgcttaggcccttaagcttaggccggcttgcttactattaacctctcataaacgctactgcaatgattggaaacttcttatagtagaatgaggcaataagacgcatctcaggtcacatatagtcttatgtttgaaacccctcactactgccatttatcttgtggaaatatctattatttcagtctatacgtaatgaaggcacttttcaggatctcttccctaagcttgtataagcaggtttgttgccgtaaccattctgtctcctcgcctaatacctgtgaagcacagaatacgtttattctataagagacgtcttaccttccatcgagattgaaagcttaaaccgtctacaacggatgccctcatcatgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacactgaattctaggctaggtatgcgaggcacgcggatctagggcagactgggcattgcatagctatggtgtagtagaactcccgtcaacggctattctcacctagactttccccttcgaactgacaagttgttatattgcctgtgtaccaagcgctaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctctgtgtattgcaccatgcagaccttcggtgcttttctcgtttccttcctcgccgccaggtaagttggccttgatgaaccatatcatatatcgccgagaagtggaccgcgtgctgagactgagacagcggcctggccgcggccgcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgcctaagtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttcttaactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatggttgcctagtgaatgctccgtaacacccaatacgccggccgaaacttttttacaactctcctatgagtcgtttacccagaatgcacaggtacacttgtttagagggtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

SEQ ID NO: 44 - DNA, 9062 bp, synthetic construct
  source 1..9062: mol_type = other DNA
cagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgcagaccttcggtgcttttctcgtttccttcctcgccgccaggtaagttggccttgatgaaccatatcatatatcgccgagaagtggaccgcgtgctgagactgagacagcggcctggccgcggccgcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgcccctacccagtctcactacggccagtgcggcggtattggctacagcggccccacggtctgcgccagcggcacaacttgccaggtcctgaacccttactactctcagtgcctgaaggtcgccgtcgagaagcgaggcgccttcaccgagaagcaggaggccctcgtctcctcctctttcgaggccttcaaggccaacatcccccagtactccgtcgtcttctacacctccatcctcgagaaggcccccgccgccaaggacctcttctctttcctctccaacggcgtcgacccttctaaccccaagctcaccggccacgccgagaagctcttcggccttgtccgtgactctgctggtcagctcaaggccaacggcaccgtcgtcgctgacgctgctcttggctctatccacgcccagaaggccatcaccgacccccagttcgtcgtcgtcaaggaggccctcctcaagaccatcaaggaggccgtcggcgacaagtggtctgacgagctctcttctgcctgggaggtcgcctacgacgagcttgccgccgccatcaagaaggccttctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

SEQ ID NO: 45 - RNA, 23 nt, synthetic construct
  source 1..23: mol_type = other RNA
gctttcgccttacttctgcaggg

SEQ ID NO: 46 - DNA, 648 bp, synthetic construct
  source 1..648: mol_type = other DNA
gtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttcttaactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatggttgcctagtgaatgctccgtaacacccaatacgccggccgaaacttttttacaactctcctatgagtcgtttacccagaatgcacaggtacacttgtttagagg

SEQ ID NO: 47 - DNA, 7935 bp, synthetic construct
  source 1..7935: mol_type = other DNA
caagcgtaactagctagcatttggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgtatcggaagttggccgtcatctcggccttcttggccacagctcgtgctggcgccttcactgagaagcaggaggccctggtcaacagcagctgggaggccttcaagggcaacatcccccagtacagcgtcgtcttctacaccagcatcctggagaaggcccctgccgccaagaacctgttcagcttcctggccaacggcgtcgaccctaccaaccctaagctgactgcccacgccgagtctctgttcggcctggtccgcgactctgctgctcagcttagggctaacggcgctgtcgtcgctgatgctgcccttggcagcatccacagccagaagggcgtcaacgacagccagttcctggtcgtcaaggaggccctgctgaagaccctgaaggaggccgtcggcgacaagtggactgacgagctttctactgccctggagctggcctacgacgagctggccgccgccatcaagaaggcctacgcctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtacactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaagtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaacagcaggcgtatttagcgga

SEQ ID NO: 48 - DNA, 3123 bp, synthetic construct
  source 1..3123: mol_type = other DNA
tgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgttgttgtcagcttggcaaattgtggctttcgaaaacacaaaacgattccttagtagccatgcattttaagataacggaatagaagaaagaggaaattaaaaaaaataaaaaaacaaacatcccgttcataacccgtagaatcgccgctcttcgtgtatcccagtaccagctacaacatcgtcctgcagttcctatatgatacgtataacccattgatacatccaagtatccagaggtgtatggaaatatcagatcaagacctctctcttctaagaaacctagaaccagacgctggtagtataataagcacactgtgactcgcttaggcccttaagcttaggccggcttgcttactattaacctctcataaacgctactgcaatgattggaaacttcttatagtagaatgaggcaataagacgcatctcaggtcacatatagtcttatgtttgaaacccctcactactgccatttatcttgtggaaatatctattatttcagtctatacgtaatgaaggcacttttcaggatctcttccctaagcttgtataagcaggtttgttgccgtaaccattctgtctcctcgcctaatacctgtgaagcacagaatacgtttattctataagagacgtcttaccttccatcgagattgaaagcttaaaccgtctacaacggatgccctcatcatgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacactgaattctaggctaggtatgcgaggcacgcggatctagggcagactgggcattgcatagctatggtgtagtagaactcccgtcaacggctattctcacctagactttccccttcgaactgacaagttgttatattgcctgtgtaccaagcgctaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctctgtgtattgcaccatgcagaccttcggtgcttttctcgtttccttcctcgccgccaggtaagttggccttgatgaaccatatcatatatcgccgagaagtggaccgcgtgctgagactgagacagcggcctggccgcggccgcggaccctccgcctgtccatgatactgatggccacgagctccgagcggacgccaactactatgtgctgagcgccaaccgagcccacggcggaggcctcaccatggcccccggccatggccgccactgccccctgttcgtgtcccaagaccctaacggccagcacgacggctttccggttcgcatcaccccctacggtgtcgccccttccgacaagattatccgcctcagcacggacgttcgcattagcttccgagcgtacactacgtgcctccagagcacggagtggcacatcgacagcgagctcgccgcgggacgccgacacgtgattaccggccctgttaaggatcccagcccctcgggtcgagagaatgctttccgcatcgagaagtactccggcgcggaggtccacgagtacaagctgatgagctgcggcgactggtgccaagacctgggcgttttccgcgacctgaagggcggtgcgtggtttctcggcgccaccgagccttaccacgtggtcgtgtttaagaaggcccctcctgcctaagtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttcttaactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatggttgcctagtgaatgctccgtaacacccaatacgccggccgaaacttttttacaactctcctatgagtcgtttacccagaatgcacaggta

SEQ ID NO: 49 - DNA, 462 bp, synthetic construct
  source 1..462: mol_type = other DNA
ggtctctcagacggcgagtggcagcttgttctcaatgcttggggcaaggtcgaagccgacgttgccggacacggccaggaggtcctcatccgattgtttaccggacatcccgagacccttgagaagttcgacaagtttaagcacctgaagacagaagcagagatgaaggcttccgaggacttgaaaaagcacggtaataccgtgctcacggcactcggcggtattctcaagaaaaagggccatcacgaggccgaggttaagcaccttgcagagtcccacgcgaacaaacacaagatccccgttaagtacctggaattcatttcagatgccattatccatgtccttcacgccaagcatccttccgatttcggcgctgacgcccaagctgccatgtcgaaggctctggagctctttcgcaacgacatggccgcacagtacaaggtccttggctttcacggctaa

SEQ ID NO: 50 - DNA, 367 bp, Trichoderma reesei
  source 1..367: mol_type = other DNA
gttgacttgcctctggtctgtccagacgggggcacgatagaatgcgggcacgcagggagctcgtagacattgggcttaatatataagacatgctatgttgtatctacattagcaaatgacaaacaaatgaaaaagaacttatcaagcactgtaccaaggaagctcattatgcgtctggcgggttcaaatgatccgtgataggttatgccagctgattgtttgcccgcagggttgacaccaccaggggataatggccactttcatctgaatcaaggacaggagccgttgatgatttccagatatcctatcttcatcgctagtactaataacaagtaagcaaacagccagctacactcgtacacaccgc

SEQ ID NO: 51 - DNA, 10239 bp, synthetic construct
  source 1..10239: mol_type = other DNA
cagcaggcgtatttagcggaggatccgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacagcttgttgaatcatggcgttccattcttcgacaagcaaagcgttccgtcgcagtagcaggcactcattcccgaaaaaactcggagattcctaagtagtgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgtgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaacaagacagaggtaagaatgtctgactcggagcgttttgcatacaaccaagggcagtgatgcaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgcagaccttcggtgcttttctcgtttccttcctcgccgccaggtaagttggccttgatgaaccatatcatatatcgccgagaagtggaccgcgtgctgagactgagacagcggcctggccgcggccggcctctctgacggcgagtggcagcttgtccttaacgcctggggcaaggtcgaggctgacgtcgctggtcacggccaggaggtccttatccgtcttttcaccggccaccccgagaccctcgagaagttcgacaagttcaagcacctcaagaccgaggccgagatgaaggcctccgaggacctcaagaagcacggcaacaccgtcctcaccgcccttggcggcatccttaagaagaagggccaccacgaggccgaggtcaagcacctcgccgagtcccacgccaacaagcacaagatccccgtcaagtacctcgagttcatctccgacgccatcatccacgtcctccacgccaagcacccctctgacttcggcgctgacgctcaggctgccatgtctaaggccctcgagctcttccgcaacgacatggccgcccagtacaaggtcctcggcttccacggctaagttgacttgcctctggtctgtccagacgggggcacgatagaatgcgggcacgcagggagctcgtagacattgggcttaatatataagacatgctatgttgtatctacattagcaaatgacaaacaaatgaaaaagaacttatcaagcactgtaccaaggaagctcattatgcgtctggcgggttcaaatgatccgtgataggttatgccagctgattgtttgcccgcagggttgacaccaccaggggataatggccactttcatctgaatcaaggacaggagccgttgatgatttccagatatcctatcttcatcgctagtactaataacaagtaagcaaacagccagctacactcgtacacaccgcgacccgtctaactcgaacatctgccacattagtctcgggtaacaggaggagtaacacgaccagtgtaacacgttaagcatacaattgaacgagaatggtgaggactgagataaaagaattctgttaaggatctaaaattatagtgcatacaaggtagatgttagtaggtggtttcagttttcctttcctttacgttggtatagagcagcgttcaccaaatgttagcagagttctatctatgtcgtatccattctgccttatatctctcaagggcgccgagctcatcctacgaagctctcaggccatcgtaggaaatacaggatagacactgaattctaggctaggtatgcgaggcacgcggatctagggcagactgggcattgcatagctatggtgtagtagaactcccgtcaacggctattctcacctagactttccccttcgaactgacaagttgttatattgcctgtgtaccaagcgctaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctctgtgtattgcaccatgtatcggaagttggccgtcatctcggccttcttggccacagctcgtgctggtctctcagacggcgagtggcagcttgttctcaatgcttggggcaaggtcgaagccgacgttgccggacacggccaggaggtcctcatccgattgtttaccggacatcccgagacccttgagaagttcgacaagtttaagcacctgaagacagaagcagagatgaaggcttccgaggacttgaaaaagcacggtaataccgtgctcacggcactcggcggtattctcaagaaaaagggccatcacgaggccgaggttaagcaccttgcagagtcccacgcgaacaaacacaagatccccgttaagtacctggaattcatttcagatgccattatccatgtccttcacgccaagcatccttccgatttcggcgctgacgcccaagctgccatgtcgaaggctctggagctctttcgcaacgacatggccgcacagtacaaggtccttggctttcacggctaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaactagtgctagacacgtataacggcacgatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataagtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagaaagactgaaaaggtggaaagaagaggcaaattgttgttattattattattctatctcgaatcttctagatcttgtcgtaaataaacaagcgtaactagctagcctccgtacaactgcttgaatttgatacccgtatggagggcagttattttattttgtttttcaagattttccattcgccgttgaactcgtctcacatcgcgtgtattgcccggttgcccatgtgttctcctactaccccaagtccctcacgggttgtctcactttctttctcctttatcctccctattttttttcaagtcagcgacagagcagtcatatggggatacgtgcaactgggactcacaacaggccatcttatggcctaatagccggcgttggcaattgcttgctttcgccagtctgttgatactctcgtgagccactccgctcacgatatcccttcattgtgcagcttccttttcccttccaattcctgctgcttccgtctcagatgcagcagagtgagttaaaagtgaggtgaactcatgggcacaagcgtccattcacctcgatccttcacttttatttcacttgcagagagtagaaccttagcaacctgaagaagaccccacacggactcggcggccccttcgcagacctgttattctcaccttcaacatcccactgatcccataccctcatactctggggatttcttcatcaccactcgaagcaaactgattcaataatcaaccttgaccaacttctgaaggacaccaattttcgcagccatgtcaactaccaaactcccaccaattactgctctcaaagccctggagccacgccccggtggccactggacagtccagggtcaggaaggggatgccatctctctttcatacgtgttgaatggcgcagaggccatgacattcgatctccgccagtgtaaaaccgaggaagatgttcgaaagttttgttcttcatttgtctacgagtacactgacaacggtatgattacttctctgaagcttatcatctcccctctaacagttctcatctctagcttctggaagtgaatggcaccatcaacctacctctgaagtccctaccgataatgtccaacctcaagaagctgcatcactgttatatcactcgcagtcacagagcaccatggggcaatactcaacagttcaaattcagcaaccgccgccacaacagcttctctttggcgctcaggcagctatggtgccgccatccgaatcgcaacctcagggaaatttgacatttggagagacggctataccttcgaactctcagttctaccaacaagaccaaaattattcgttacctaatggccaaggcctttcacatgacagtttcgctcctggaactggcgagtggccactaggcaatgagattttccccgtaggcgtaggagaaattcctatttctatcccgaacccggaactcggaataactcaagacgatactatgttcttgggtgcgtggcatttaaatggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttaaattgtgcgagatccatgactgcgttgactgatggtgcgggcttgctctggtaatatatcgcgagaagatgggatatgaacgttgatgcaggagttggaagataggaggaacttgagaggtaggaagttttcggtcgcctaggtaatgctttgaagaattgaaaaggctgtgggtggagggtgagctacaagtgagccggaacgtgccgtggtgattcaacttactggcctttattgatgagagctgccaacccaaagcgagtgaatctgaatagtgcccccccagcccctccgttatttagcgccaggtcagtgcgcgctccccacctatctctcttttctcaccctccctccttggggaaaaatgactaggtactactgatatgtaagaaggtaaggtagaaacctaggtactactaggtaatatgtatttctgcttgccaacgacaagctacctcctaaggaaccttgcaagcgtacctaggtaccttctaaaggaagtgttggggctagaatctagcagtcccacccgcccatcgccagagggtgtgcatcttcgtaacaatacgcccaccgtggagatatcctcatttcgcacaccagcaaaatgctctccaggtgtgcatgacgttcgaggcagtacttcacccaagatgcgcgtgaagtttagctttgggaacaacctgaaagttcgtgaaggacatttatgtacataccatttgcatcaaatcagaatatatacgatctccatggaaaggataagatcaagcaacatgtcttcgtccgttccgccagagagcgcaatcacttatgccaatggtttcacacaaatgcagttgcagtttatcactttccaggggcgcaatgttgctgggacatctagttgcgaaagctcgattctgttatttcatacgattcagatctgagattccaaacggacagtgtacgatccatgaagaatttcaacatggaggtagttcatgacaacgtttgacttgaaatactggcagaagctgaaattgatcttggcaagagccaacacaaa

(19) United States
(12) Patent Application Publication
(10) Pub. No.: US 2026/0226401 A1
(43) Pub. Date: Aug. 6, 2026

(54) COMPOSITIONS AND METHODS FOR PRODUCING HETEROLOGOUS GLOBINS IN FILAMENTOUS FUNGAL CELLS

(71) Applicant: DANISCO US INC., Palo Alto, CA (US)

(72) Inventors: Lixuan Lisa Huang, Palo Alto, CA (US); Cherry Lin, Palo Alto, CA (US); Michael J. Pepsin, CastroValley, CA (US); Chuanbin Liu, Menlo Park, CA (US)

(21) Appl. No.: 19/154,937

(22) PCT Filed: Jan. 26, 2024
(86) PCT No.: PCT/US2024/013133

§ 371 Date: Aug. 8, 2025

Related U.S. Application Data
(60) Provisional application No. 63/483,859, filed on Feb. 8, 2023.

(51) Int. Cl. C12N 1/14; C07K 14/805; C07K 14/81; C12N 15/80; C12R 1/645

(52) U.S. Cl. CPC C12N 1/14; C07K 14/805; C07K 14/81; C12N 15/80; C12R 2001/645

(57) ABSTRACT

The present disclosure is generally related to methods and compositions for producing heterologous globin proteins of interest in recombinant filamentous fungal cells. Certain embodiments are therefore, directed to compositions and methods for the production of globin proteins, recombinant filamentous fungal strains comprising enhanced globin protein productivity phenotypes, polynucleotides (e.g., expression constructs) encoding one or more globin proteins, industrial scale fermentation, globin protein recovery processes and the like.

### FIELD

The present disclosure is generally related to the fields of biology, microbiology, molecular biology, filamentous fungi, food proteins, industrial protein production the like. Certain embodiments are related to methods and compositions for producing heterologous globin proteins in filamentous fungal strains. As described herein, the recombinant fungal strains of the disclosure are particularly well-suited for growth in submerged cultures for the large-scale production of heterologous globin proteins.

### CROSS REFERENCE TO RELATED APPLICATIONS

This application claims benefit to U.S. Provisional Patent Application No. 63/483,859, filed Feb. 8, 2024, which is incorporated herein by referenced in its entirety.

### REFERENCE TO A SEQUENCE LISTING

The contents of the electronic submission of the text file Sequence Listing, named “NB42149-WO-PCT_SequenceListing.xml” was created on Jan. 23, 2024 and is 227 KB in size, which is hereby incorporated by reference in its entirety.

### BACKGROUND

As appreciated by one skilled in the relevant arts, over the past 10-15 years there has been an ongoing shift to develop and produce plant-based meat substitutes which have similar taste and/or aroma profiles to animal-based meats. In particular, as described in PCT Publication WO2013/010042, animal farming has a profound negative environmental impact, wherein an estimated 30% of Earth's land surface is dedicated to animal farming, such that livestock account for more than 20% of total terrestrial animal biomass. As further described in the WO2013/010042 publication, due to the massive scale animal farming, such practices account for more than 18% of net greenhouse gas emissions, suggesting that animal farming may be the largest source of water pollution and the world's largest threat to biodiversity. For example, it has been estimated that if the human population could shift from a meat containing diet to a diet free of animal products (vegetarian diet), more than 26% of Earth's land surface would be freed up for other uses and massively reduce water and energy consumption.

In certain aspects, the WO2013/010042 publication speculates that one or more plant-based (meat) proteins may be isolated and purified from genetically modified organisms (e.g., genetically modified bacteria or yeast cells), wherein the one or more isolated and purified plant proteins include hemoglobins, myoglobins, leghemoglobins, non-symbiotic hemoglobins, and the like. For example, the leghemoglobin protein derived from soybean is a key food additive that imparts meaty flavor and color to meat analogues. In particular, the WO2013/010042 specification and experimental examples section describe the construction of a muscle replica (composition), a muscle tissue analogue, a fat tissue analogue, and a connective tissue analogue, wherein the muscle replica/tissue analogues were each constructed from one or more plant proteins (hemoglobins, myoglobins, leghemoglobins, etc.) isolated and purified from the one or more native plant source(s), i.e., as opposed to being expressed and recovered from a genetically modified organism.

PCT Publication No. WO2014/110532 describes methods and compositions for modulating the flavor and aroma profiles of consumable food products using so-called “plant-based meat substitutes” having properties similar to animal-based meat compositions, wherein the plant-based meat substitutes contain one or more flavor precursors (e.g., sugars, oils, FFAs, amino acids, nucleosides, vitamins, etc.) and one or more highly conjugated heterocyclic rings complexed to an iron complex (i.e., heme prosthetic group). US Patent Publication No. US2014/0161958 describes a meat substitute product comprising a vegetable protein blended with a starch, a hydrocolloid, and an oil from a vegetable source. US Patent Publication No. US2021/0289813 describes meat substitutes comprising two or more sources of plant protein, or meat substitutes comprising one or more sources of plant protein and a fruit, fruit powder, or chia seed extract, or a low allergen meat substitute that is optionally free of soy and optionally free of other allergenic ingredients.

In other aspects, attempts to produce certain globin proteins (e.g., leghemoglobins, cyanoglobins) in recombinant microbial host cells (e.g., E. coli, cyanobacteria, yeast) have been described. In certain aspects, PCT Publication No. WO2016/183163 generally describes methods for constructing modified methylotrophic yeast cells (P. pastoris) for expression of recombinant proteins, wherein the modified yeast (cells) co-express the entire heme biosynthetic pathway from methanol inducible promoters. For instance, the WO2016/183163 publication teaches the use of P. pastoris strains overexpressing the transcriptional activator Mxr1 under the control of the alcohol oxidase 1 (AOX1) promoter element to increase expression/co-expression of recombinant proteins and the heme biosynthetic pathway.

However, as reviewed in Kramer et al. (2015), insufficient incorporation of heme is considered a central impeding cause in the recombinant production of active heme proteins. In particular, Kramer et al. (2015) investigated the effects of both pathway engineering, and medium supplementation, to optimize the recombinant production of the heme protein horseradish peroxidase (HRP) in the yeast P. pastoris. As summarized by Kramer et al. (2015), in contrast to other studies, (a) co-overexpression of genes of the endogenous heme biosynthesis pathway in P. pastoris did not improve the recombinant production of active heme (HRP) enzyme, (b) medium supplementation with the commonly used precursor 5-aminolevulonic acid (ALA) did not affect yield of the active heme (HRP) enzyme, whereas (c) medium supplementation with hemin increased the yield of active heme (HRP) enzyme, concluding that the yield of active peroxidase enzyme from P. pastoris can be easily enhanced by supplementation of the cultivation medium with hemin.

PCT Publication No. WO2019/079135 generally describes non-animal derived meat-like materials/ingredients obtained from genetically modified cyanobacteria comprising polynucleotides encoding heterologous globin proteins (e.g., leghemoglobins, cyanoglobins). PCT Publication No. WO2023/278968 describes non-heme iron-binding protein pigment compositions for meat substitutes which provide a pink and/or red color to the meat substitute composition.

Despite current knowledge related to globin proteins, their production and/or uses and applications, there remain ongoing and unmet needs in the art. In particular, due to growing demands for plant-based meat substitutes, plant-based (meat) proteins, non-animal derived meat-like products and the like, there continue to be ongoing and unmet needs in the art for enhanced globin (protein) expression systems suitable for cost-efficient large-scale production of globin proteins.

### SUMMARY

As described hereinafter, the instant disclosure addresses ongoing and unmet needs in the art related to the production of heterologous globin proteins. More particularly, as set forth and described herein, certain one or more embodiments of the disclosure provide, inter alia, novel methods and compositions for the production of globin proteins, recombinant filamentous fungal strains having enhanced globin protein productivity phenotypes, polynucleotides (e.g., expression cassettes) encoding globin proteins, polynucleotide (linker DNA) sequences encoding protein/amino acid cleavage sites, industrial scale fermentation processes and the like. Thus, certain one or more embodiments are directed to recombinant filamentous fungal cells capable of producing heterologous globin proteins for use in, inter alia, food materials, food ingredients, flavor modifiers, aroma modifiers and the like.

In certain embodiments, the disclosure is related to recombinant filamentous fungal cells expressing heterologous globin proteins. In related embodiments, the disclosure provides recombinant filamentous fungal cells expressing and secreting heterologous globin proteins into the fermentation broth when fermented under suitable conditions. In one or more other embodiments, recombinant filamentous fungal cells comprise introduced expression cassettes encoding the globin proteins.

In certain embodiments, expression cassettes comprise at least an upstream promoter (pro) sequence operably linked to a downstream nucleic acid (sig-seq) encoding a protein (signal) secretion sequence operably linked to a downstream (3′) nucleic acid (globin CDS) encoding the globin protein (e.g., 5′-[pro]-[sig-seq]-[globin CDS]). In other related embodiments, the nucleic acid (globin CDS) encoding the globin protein may comprise an upstream nucleic acid (N-fusion) encoding a N-terminal protein fusion and/or an upstream nucleic acid (N-linker) encoding a N-terminal protein cleavage site and/or a downstream nucleic acid (C-fusion) encoding a C-terminal protein fusion and/or a downstream nucleic acid (C-linker) encoding a C-terminal protein cleavage site, and/or an upstream nucleic acid (N-fusion) encoding a N-terminal protein fusion and/or a downstream nucleic acid (C-fusion) encoding a C-terminal protein fusion and combinations thereof.

In certain other embodiments, the one or more expression cassettes are integrated into the genome of the cell. In other embodiments, the recombinant filamentous fungal cells comprise one or more introduced expression cassettes encoding one or more protease inhibitor proteins. In other embodiments, the recombinant fungal cells are fermented for at least about 96 hours to about 300 hours for the production of the globin protein. In related embodiments, the recombinant fungal cells are fermented for at least about 96 hours to about 300 hours and produce at least 0.1, 0.2, 0.3, 0.4 or 0.5 grams of globin protein per liter of fermentation broth (g/L). In certain other embodiments, the recombinant fungal cells are fermented for about 180-190 hours and produce at least 1.0 grams of globin protein per liter of fermentation broth (g/L). In related embodiments, the globin proteins produced are selected from the group consisting of leghemoglobins, myoglobins, hemoglobins, cyanoglobins and non-symbiotic hemoglobins.

In other embodiments, the disclosure provides methods for producing heterologous globin proteins in filamentous fungal cell. In certain embodiments, the methods include, but are not limited to, introducing an expression cassette encoding a globin protein into the filamentous fungal cell, wherein the cassette comprises at least an upstream promoter (pro) sequence operably linked to a downstream nucleic acid (sig-seq) encoding a protein (signal) secretion sequence operably linked to a downstream (3′) nucleic acid (globin CDS) encoding the globin protein and fermenting the modified cell under suitable conditions for the production of the globin protein. In certain embodiments of the methods, the filamentous fungal cells are selected from the group consisting of Acremonium sp. cells, Aspergillus sp. cells, Emericello sp. cells, Fusarium sp. cells, Humicola sp. cells, Mucor sp. cells, Myceliophthora sp. cells, Neurospora sp. cells, Penicillium sp. cells, Scytalidium sp. cells, Talaromyces sp. cells, Thielavia sp. cells, Tolypocladium sp. cells and Trichoderma sp. cells. In other embodiments of the methods, one or more expression cassettes encoding globin proteins are integrated into the genome of the cell. In other embodiments of the methods, the fungal cells comprise an introduced expression cassette encoding at least two globin proteins and/or comprise at least two introduced expression cassette encoding at least two globin proteins. In certain preferred embodiments of the methods, the recombinant fungal cells comprise an introduced expression cassette encoding a protease inhibitor. In yet other embodiments of the methods, the recombinant fungal cells are fermented for at least about 96 hours to about 300 hours and produce at least 0.1, 0.2, 0.3, 0.4 or 0.5 grams of globin protein per liter of fermentation broth (g/L). In certain other embodiments, the fungal cells are fermented for about 180-190 hours and produce at least 1 grams of globin protein per liter of fermentation broth (g/L). In yet other embodiments of the methods, the expressed globin is secreted and recovered from the fermentation broth, wherein the recovered globin protein is optionally purified. Thus, in certain other embodiments of the methods, the secreted globin protein is selected from the group consisting of leghemoglobins, myoglobins, hemoglobins, cyanoglobins and non-symbiotic hemoglobins.

### BRIEF DESCRIPTION OF DRAWINGS

FIG. 1 shows the amino acid and codon optimized DNA sequences encoding exemplary globin proteins. In particular, FIG. 1 presents the amino acid sequences of a native soybean leghemoglobin (SEQ ID NO: 1) encoded by DNA of SEQ ID NO: 2, a native bovine myoglobin (SEQ ID NO: 3) encoded by DNA of SEQ ID NO: 4, and a native French bean leghemoglobin (SEQ ID NO: 18) encoded by DNA of SEQ ID NO: 19.

FIG. 2 presents the amino acid sequences of a native BASI protein (SEQ ID NO: 5), a codon optimized DNA sequence (SEQ ID NO: 6) encoding the native BASI protein, the Cbh1 core domain protein (SEQ ID NO: 7) containing the seventeen (17) amino acid signal peptide at the N-terminus, and the full-length, Cbh1 protein containing the 17 amino acid signal peptide at the N-terminus (SEQ ID NO: 9).

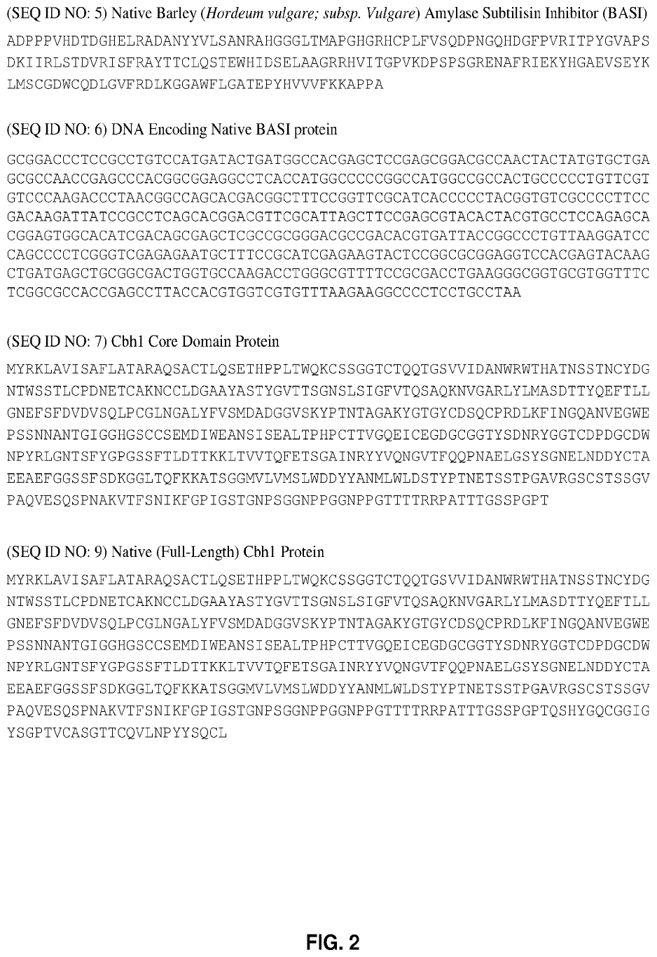

FIG. 3 shows centrifuged culture broths harvested at twenty-four (24) hour intervals in the fed-batch fermentation of T. reesei strain BFZ28, which expresses/produces the soybean leghemoglobin by secretion. As shown in FIG. 3, fermentation broth samples were collected at 21 hours, 48 hours, 67 hours, 91 hours, 120 hours, 143 hours, 167 hours, and 188 hours from the start of the fermentation run.

FIG. 4 shows the total secreted protein titers from the fermentation run of strain BFZ28 (e.g., see FIG. 3). The titer represents the presence of total soluble proteins present in the culture supernatant, including the CBH1 core domain protein, the soybean leghemoglobin protein and other background proteins secreted by the T. reesei host strain.

FIG. 5 shows an SDS-PAGE analysis for the BFZ28 strain fermentation run, wherein molecular weight markers (kDa) are shown on the left of the gel and the Cbh1 core protein, leghemoglobin protein/BASI protease inhibitor are shown with labels on the right side of the gel. More particularly, as presented in FIG. 5, the CBH1 core protein has an approximate molecular weight (Mw) of about 49 kDa, the leghemoglobin protein has an approximate Mw of about 15.5 kDa, and the BASI protease inhibitor has an approximate Mw of about 20 kDa.

FIG. 6 shows the HPLC analysis of the 188-hour supernatant sample from the BFZ28 strain fermentation run. In particular, the heme group of the leghemoglobin protein was detected at a wavelength of 410 nm (heme prosthetic group), which co-migrated with a protein peak detected at 280 nm with a 6.4-minute retention time. The insert drawing (FIG. 6) shows the spectral scan of the leghemoglobin protein, with a peak at about 6.4 minutes retention time (wavelength 410 nm), confirming the peak absorbance at 410 nm for the heme containing leghemoglobin protein.

FIG. 7 shows the total secreted protein titers from fermentation runs of strains BFZ28 (Pcbh1-CBH1 core-KEX2-LegGm1b), BGJ74 (Pcbh1-LegGm1b, Pcbh2-BASI), BGJ75 (Pcbh1-Pv1b, Pcbh2-BASI), and BGJ76 (Pcbh1-LegGm1b.Pep1ss, Pcbh2-BASI). The titer represents the presence of total soluble proteins present in the culture supernatant, including the CBH1 core domain protein (in BFZ28), the BASI protein (in BGJ74, BGJ75, and BGJ76), the soybean leghemoglobin protein (in BFZ28, BGJ74, BGJ75, and BGJ76) and other background proteins secreted by the T. reesei host strain.

FIG. 8 shows SDS-PAGE analysis for the fermentation runs of strains BGJ74, BGJ75, and BGJ76. As shown in FIG. 8, the red arrow indicates the BASI and leghemoglobin bands co-migrating at approximately 20 kDa.

FIG. 9 shows the chromatogram of the HPLC analysis for the 188-hour supernatant samples from the 188-hour end-of-fermentation runs of strains BGJ74, BGJ75, and BGJ76. As presented in FIG. 9, the protein peaks are detected at 280 nm. The heme prosthetic group of the leghemoglobin protein is detected at 410 nm in FIG. 9, which co-migrated with a protein peak detected at 280 nm with a 6.4-minute retention time.

FIG. 10 shows the SDS-PAGE gel analysis of the fermentation run of strain BHX46. The first sample lane (labeled “C”) contains equine myoglobin. The subsequent lanes contain the culture supernatant samples harvested at 43 h, 72 h, 94 h, 114 h, 137 h, 161 h and 186 h.

### BRIEF DESCRIPTION OF THE BIOLOGICAL SEQUENCES

SEQ ID NO: 1 is the amino acid sequence of a native Glycine max (soybean) leghemoglobin protein (NCBI Accession: NP_001235248).

SEQ ID NO: 2 is a nucleic acid (DNA) sequence encoding the native leghemoglobin protein of SEQ ID NO: 1, wherein SEQ ID NO: 2 has been codon optimized for expression in T. reesei fungal cells.

SEQ ID NO: 3 is the amino acid sequence of a native Bos taurus (bovine) myoglobin protein (NCBI Accession: NP_776306.1; GI: 27806939).

SEQ ID NO: 4 is a DNA sequence encoding the native myoglobin protein of SEQ ID NO: 3, wherein SEQ ID NO: 4 has been codon optimized for expression in T. reesei fungal cells.

SEQ ID NO: 5 is the amino acid sequence of a native barley amylase subtilisin inhibitor (BASI) protein (NCBI Accession: 1210227A).

SEQ ID NO: 6 is a DNA sequence encoding the native BASI protein of SEQ ID NO: 5, wherein SEQ ID NO: 6 has been codon optimized for expression in T. reesei fungal cells.

SEQ ID NO: 7 is the amino acid sequence of a Cbh1 core domain protein.

SEQ ID NO: 8 is a DNA sequence encoding the Cbh1 core domain.

SEQ ID NO: 9 is the amino acid sequence of the native (full-length) Cbh1 protein (NCBI Accession: A0A024RXP8).

SEQ ID NO: 10 is a DNA sequence encoding the full-length Cbh1 protein.

SEQ ID NO: 11 is a cbh1 promoter region (DNA) sequence of the T. reesei chb1 gene.

SEQ ID NO: 12 is a cbh2 promoter region (DNA) sequence of the T. reesei chb2 gene.

SEQ ID NO: 13 is a DNA sequence encoding the Cbh1 signal peptide sequence.

SEQ ID NO: 14 is a DNA sequence encoding the signal peptide sequence of a Pep1 protein.

SEQ ID NO: 15 is a terminator (DNA) sequence of the cbh1 gene.

SEQ ID NO: 16 is a T. reesei pyr2 gene (marker) encoding for orotate phosphoribosyl transferase.

SEQ ID NO: 17 is a DNA sequence encoding an A. nidulans acetamidase.

SEQ ID NO: 18 is a DNA sequence encoding a Kex2 protease cleavage site.

SEQ ID NO: 19 is the amino acid sequence of a native French bean (Phaseolus vulgaris) leghemoglobin (NCBI Accession: AAA337671).

SEQ ID NO: 20 is a DNA sequence encoding the native leghemoglobin protein of SEQ ID NO: 19, wherein SEQ ID NO: 20 has been codon optimized for expression in T. reesei fungal cells.

SEQ ID NO: 21 is a synthetic DNA primer sequence named OT4268.

SEQ ID NO: 22 is a synthetic DNA primer sequence named OT4269.

SEQ ID NO: 23 is a synthetic DNA primer sequence named OT4337.

SEQ ID NO: 24 is a synthetic DNA primer sequence named OT4338.

SEQ ID NO: 25 is a synthetic DNA primer sequence named OT4333.

SEQ ID NO: 26 is a synthetic DNA primer sequence named OT4334.

SEQ ID NO: 27 is an integration cassette named HG1.

SEQ ID NO: 28 is an integration cassette named HG2.

SEQ ID NO: 29 is an integration cassette named HG3.

SEQ ID NO: 30 is an integration cassette named HG4.

SEQ ID NO: 31 is an integration cassette named HG5.

SEQ ID NO: 32 is an integration cassette named HG6.

SEQ ID NO: 33 is an integration cassette named HG7.

SEQ ID NO: 34 is an integration cassette named HG8.

SEQ ID NO: 35 is an integration cassette named HG9.

SEQ ID NO: 36 is a synthetic cassette named pCHL853.

SEQ ID NO: 37 is a synthetic cassette named pCHL856.

SEQ ID NO: 38 is a synthetic cassette named pLH1088.

SEQ ID NO: 39 is a synthetic cassette named pLH1104.

SEQ ID NO: 40 is a synthetic cassette named pLH1105.

SEQ ID NO: 41 is a synthetic cassette named pLH1106.

SEQ ID NO: 42 is a synthetic cassette named pLH1107.

SEQ ID NO: 43 is a synthetic cassette named pLH1108.

SEQ ID NO: 44 is a synthetic cassette named pLH1109.

SEQ ID NO: 45 is a synthetic single guide RNA named “sgRNA-TrC144F”.

SEQ ID NO: 46 is a synthetic DNA sequence comprising a TrpC transcriptional terminator sequence.

SEQ ID NO: 47 is a synthetic cassette named pCHL852.

SEQ ID NO: 48 is an integration cassette named HG10.

SEQ ID NO: 49 is a second codon optimized bovine myoglobin gene (Mb1c) encoding the bovine same myoglobin protein of SEQ ID NO: 3.

SEQ ID NO: 50 is a synthetic DNA comprising a T. reesei Egl1 terminator (Tegl1)

SEQ ID NO: 51 is a tandem-copy expression vector named “pLHX143”.

### DETAILED DESCRIPTION

As described herein, certain embodiments of the disclosure provide, inter alia, compositions and methods for the production of globin proteins, recombinant filamentous fungal strains comprising enhanced globin protein productivity phenotypes, polynucleotides (e.g., expression constructs) encoding one or more globin proteins, industrial scale fermentation and recovery processes of globin proteins and the like. Thus, in one or more embodiments or aspects, the disclosure is related to recombinant (modified) filamentous fungal cells capable of producing heterologous globin proteins. In certain embodiments or aspects, recombinant filamentous fungal cells comprise introduced expression cassettes encoding one or more heterologous globin proteins of interest. In certain embodiments, one or more cassettes comprise an upstream (5′) promoter (pro) region sequence operably linked to a downstream nucleic acid (sig-seq) encoding a protein secretion (signal) sequence operably linked to downstream (3′) nucleic acid (globin CDS) encoding a globin protein. In other one or more embodiments or aspects, recombinant filamentous fungal cells comprising one or more introduced cassettes are fermented under suitable conditions for the production the globin protein. In certain other embodiments, the secreted globin proteins are recovered from the end of fermentation (EOF) broth. More particularly, as set forth and described hereinafter, the recombinant fungal strains of the instant disclosure are particularly well-suited for growth in submerged cultures for the large-scale production of heterologous globin proteins.

### I. Definitions

Prior to describing the present strains and methods in detail, the following terms are defined for clarity. Terms not defined should be accorded their ordinary meanings as used in the relevant art. Unless defined otherwise, all technical and scientific terms used herein have the same meaning as commonly understood by one of ordinary skill in the art to which the present compositions and methods apply.

All publications and patents cited in this specification are herein incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. For example, in connection with a numerical value, the term “about” refers to a range of ⁻10% to ⁺10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

In accordance with this Detailed Description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely”, “only”, “excluding”, “not including” and the like in connection with the recitation of claim elements, or use of a “negative” limitation.

It is further noted that the term “comprising”, as used herein, means “including, but not limited to”, the component(s) after the term “comprising”. The component(s) after the term “comprising” are required or mandatory, but the composition comprising the component(s) may further include other non-mandatory or optional component(s).

It is also noted that the term “consisting of,” as used herein, means “including and limited to”, the component(s) after the term “consisting of”. The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the terms “wild-type” and “native” are used interchangeably and refer to genes, proteins, fungal cells or strains as found in nature.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell, or may provide an altered expression pattern of endogenous genes, such as being over-expressed, under-expressed, minimally expressed, or not expressed at all.

“Recombination”, “recombining” or generating a “recombined” nucleic acid is generally the assembly of two or more nucleic acid fragments wherein the assembly gives rise to a chimeric gene.

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the term “gene” means the segment of DNA involved in producing a polypeptide (protein) chain, that may or may not include regions preceding and following the coding region (e.g., 5′ untranslated (5′ UTR) or “leader” sequences, 3′ UTR or “trailer” sequences, promoter sequences, terminator sequences and the like) as well as intervening sequences (introns) between individual coding segments (exons). For example, a gene (DNA) sequence of interest (GOI) may encode a globin protein of interest, a structural protein, commercially important industrial proteins or peptides, such as enzymes (e.g., proteases, mannanases, xylanases, amylases, glucoamylases, cellulases, oxidases, phytases, lipases) and the like. The gene of interest may be a naturally occurring gene, a mutated (modified) gene or a synthetic gene.

As used herein, the term “promoter” refers to a nucleic acid sequence that functions to direct transcription of a downstream gene, or an open reading frame (ORF) thereof. The promoter will generally be appropriate to the host cell (e.g., a filamentous fungal cell) in which the target gene is being expressed. The promoter together with other transcriptional and translational regulatory nucleic acid sequences (also termed “control sequences”) is necessary to express a given gene. In general, the transcriptional and translational regulatory sequences include, but are not limited to, promoter and terminator sequences including a core promoter and enhancer or activator or repressor sequences, transcriptional and translational start and stop sequences. In certain embodiments, the promoter is an inducible promoter, or a constitutive promoter. In certain embodiments, the inducible promoter is an inducible cellulase gene promoter.

As used herein, the term “promoter activity” is the ability of a nucleic acid to direct transcription of a downstream (3′) polynucleotide in a host cell. To test promoter activity, the (promoter) nucleic acid may be operably linked to a downstream polynucleotide to produce a recombinant nucleic acid. The recombinant nucleic acid may be introduced into a cell, and transcription of the polynucleotide may be evaluated. In certain cases, the polynucleotide may encode a protein, and transcription of the polynucleotide can be evaluated by assessing production of the protein in the cell.

As used herein, the term “operably linked” refers to a functional linkage between two or more nucleic acid sequences. Thus, a nucleic acid sequence is operably linked when it is placed into a functional relationship with another nucleic acid sequence. For example, a promoter sequence or a terminator sequence is operably linked to a coding sequence if it affects the transcription of the coding sequence; a ribosome binding site is operably linked to a coding sequence if it is positioned so as to facilitate translation; a nucleic acid sequence encoding a secretory leader (i.e., a signal peptide) is operably linked to a nucleic acid sequence (e.g., an ORF) encoding a polypeptide if it is expressed as a pre-protein that participates in the secretion of the polypeptide. Generally, “operably linked” means that the DNA (nucleic acid) sequences being linked are contiguous, and, in the case of a secretory leader, contiguous and in reading phase. However, enhancers do not have to be contiguous. Linking two or more nucleic acid sequences (i.e., operably linking) is accomplished using any of the methods to one of skill in the art.

As used herein, a “functional gene” is a gene capable of being used by cellular components to produce an active gene product, typically a protein. In contrast, a “non-functional gene” cannot be used by cellular components to produce an active gene product (i.e., a functional protein), or has a reduced ability to be used by cellular components to produce an active gene product (i.e., a functional protein).

As used herein, a “functional protein” is a protein that possesses a function or activity, such as an enzymatic function/activity, a binding function/activity (e.g., DNA binding), a surface-active property, and the like, and which has not been mutagenized, truncated, or otherwise modified to abolish or reduce that function/activity.

As used herein, the phrases “modified filamentous fungal cell(s)”, “mutant or variant filamentous fungal cell(s)”, “recombinant fungal cell(s)”, “modified filamentous fungal strain(s)”, and the like may be used interchangeably and refer to filamentous fungal cells that are derived (obtained) from a control or parental filamentous fungal cell belonging to the Pezizomycotina subphylum. For example, a “modified” filamentous fungal cell may be derived (obtained) from a control or parental filamentous fungal cell, wherein the modified cell comprises at least one genetic modification which is not found in the control or parental cell.

As used herein, the term “Ascomycete fungal cell” refers to any organism in the Division Ascomycota in the Kingdom Fungi. Examples of Ascomycetes fungal cells include, but are not limited to, filamentous fungi in the subphylum Pezizomycotina, such as Trichoderma sp., Aspergillus sp., Myceliophthora sp. and Penicillium sp.

As used herein, the term “filamentous fungus” refers to all filamentous forms of the subdivision Eumycota and Oomycota. For example, filamentous fungi include, without limitation, Acremonium, Aspergillus, Emericella, Fusarium, Humicola, Mucor, Myceliophthora, Neurospora, Penicillium, Scytalidium, Talaromyces, Thielavia, Tolypocladium, or Trichoderma species. In some embodiments, the filamentous fungus may be an Aspergillus aculeatus, Aspergillus awamori, Aspergillus foetidus, Aspergillus japonicus, Aspergillus nidulans, Aspergillus niger, or Aspergillus oryzae.

In some embodiments, the filamentous fungus is a Fusarium sp. such as Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, and the like. In other embodiments, the filamentous fungus is Humicola insolens, Humicola lanuginosa, Mucor miehei, Myceliophthora thermophila, Neurospora crassa, Scytalidium thermophilum, Thielavia terrestris and the like. In certain other embodiments, a filamentous fungus is a Trichoderma harzianum, Trichoderma koningii, Trichoderma longibrachiatum, Trichoderma reesei, Trichoderma viride and the like.

As used herein, exemplary parental Trichoderma reesei strains include, but are not limited to, T. reesei strain QM6a (ATCC Deposit No. 13631), T. reesei strain RL-P37 (NRRL Deposit No. 15709) and T. reesei strain RUT-C30 (ATCC Deposit No. 56765); exemplary parental Aspergillus niger strains include, but are not limited to, A. niger strain designated as ATCC Deposit No. 1015; exemplary parental Aspergillus oryzae strains include, but are not limited to A. oryzae strain RIB40 (ATCC Deposit No. 42149); and exemplary parental Myceliophthora thermophila strains include, but are not limited to, M. thermophila strain designated as ATCC Deposit No.42464. For example, Trichoderma strains RUT-C30 and RL-P37 are mutagenized (cellulase overproducing) derivatives of Trichoderma natural isolate QM6a (Sheir-Neiss and Montenecourt, 1984), with strain NG14 being the last common ancestor. In certain aspects, suitable Trichoderma strains may be derived/obtained from T. reesei strains comprising a deletion of the T. reesei pyr2 gene (Δpyr2), as generally described by Sheir-Neiss and Montenecourt (1984) and PCT Publication No. WO2011/153449 (specifically incorporated herein by reference in its entirety). In certain embodiments one or more embodiments, T. reesei cells/strains of the disclosure are obtained/derived from T. reesei strain RL-P37. Thus, in certain one or more embodiments, T. reesei cells derived from strain RL-P37 are abbreviated herein as “Tr1 cells”, “Tr1 strains”, “Tr1 parental” cells, “Tr1 control” cells and the like.

As used herein, the phrases “lignocellulosic degrading enzymes”, “cellulase enzymes”, and/or “cellulases” are used interchangeably, and include glycoside hydrolase (GH) enzymes, such as cellobiohydrolases, xylanases, endoglucanases, and β-glucosidases, that hydrolyze the β-(1,4)-linked glycosidic bonds of cellulose (hemi-cellulose) to produce glucose.

In certain embodiments, cellobiohydrolases include enzymes classified under Enzyme Commission No. (EC 3.2.1.91), endoglucanases include enzymes classified under EC 3.2.1.4, endo-β-1,4-xylanases include enzymes classified under EC 3.2.1.8, β-xylosidases include enzymes classified under EC 3.2.1.37, and β-glucosidases include enzymes classified under EC 3.2.1.21.

As used herein, “endoglucanase” proteins may be abbreviated as “EG”, “cellobiohydrolase” proteins may be abbreviated “CBH”, “β-glucosidase” proteins may be abbreviated “BG” and “xylanase” proteins may be abbreviated “XYL”. Thus, as used herein, a gene (gene CDS or ORF) encoding a EG protein may be abbreviated “eg”, a gene (or ORF) encoding a CBH protein may be abbreviated “cbh”, a gene (or ORF) encoding a BG protein may be abbreviated “bg”, and a gene (or ORF) encoding a XYL protein may be abbreviated “xyl”.

As used herein, “globins” or “globin proteins” are metalloproteins comprising a “porphyrin prosthetic group”. In particular, globin proteins incorporate a series of α-helical segments known as globin folds, which accommodate/bind the porphyrin prosthetic group. Globin proteins include, but are not limited to, “leghemoglobin”, “myoglobin” and “hemoglobin”. In certain aspects, the porphyrin prosthetic group confers globin protein functionality, which functionality can include oxygen carrying or transport, oxygen reduction, electron transfer, and other processes.

As used herein, the term “porphyrins” has the same meaning as understood in the art, wherein porphyrins are a group of heterocyclic macrocycle organic compounds composed of four modified pyrrole subunits interconnected at their a carbon atoms via methine bridges. In particular, the porphyrin (ring structure) is often described as a highly conjugated aromatic, which strongly absorbs electromagnetic radiation in the visible region of the spectrum. For example, with a concomitant displacement of two (2) N—H protons, porphyrins bind metal ions in the N4 pocket, wherein the metal ions usually have a charge of 2⁺ or 3⁺. In particular, when there is no metal ion (or atom) bound to the nitrogens in the ring (structure) center, the compounds are referred to as “free porphyrins”, whereas if they are bonded to a metal ion (or atom) in the ring (structure) center, they are referred to as “bound porphyrins”. Examples of porphyrins with bound iron atoms include, but are not limited to, myoglobin and hemoglobin. In particular, one of the best-known families of porphyrin complexes are hemes.

As used herein, the term “hemin” refers to a porphyrin (protoporphyrin IX) comprising a ferric iron (Fe³⁺) ion with a coordinating chloride ligand.

As used herein, phrases such as “retaining globin protein function or activity”, “comprising globin protein function or activity”, and the like means the heme (porphyrin) is bound to the globin protein's heme (porphyrin) binding pocket, either in a penta-coordinated state for some globins (e.g., leghemoglobin), or in a hexa-coordinated state for other globins (e.g., cyanoglobin). Thus, in certain one or more embodiments or aspects of the disclosure, a functional globin protein may be assayed and detected according to the bound heme (porphyrin) prosthetic group, which can be measured/detected based on the UV-Vis absorbance of the porphyrin molecule. For example, the bound heme set forth in the instant examples has a signature UV-Vis absorption peak at 410 nm.

As used herein, a “native soybean leghemoglobin protein” comprises at least about 90% to 100% identity the native Glycine max (soybean) leghemoglobin protein of SEQ ID NO: 1. In certain embodiments, a native soybean leghemoglobin protein comprises at least about 90% to 100% identity to the native soybean leghemoglobin protein of SEQ ID NO: 1 and retains (comprises) native leghemoglobin function or activity.

As used herein, a “gene coding sequence (CDS)” encoding native soybean leghemoglobin protein encodes a leghemoglobin protein comprising at least about 40% to 100% identity the native protein of SEQ ID NO: 1. In certain one or more embodiments, a gene CDS encoding a native soybean leghemoglobin protein comprises at least about 90% to 100% identity to the SEQ ID NO: 1 and retains native leghemoglobin function or activity. In certain embodiments, the wild-type (WT) soybean leghemoglobin C2 gene CDS of the disclosure is abbreviated “LegGm1b”, wherein the WT LegGm1b gene CDS has been codon optimized for expression in T. reesei cells, as set forth in SEQ ID NO: 2.

As used herein, a “native French bean leghemoglobin protein” comprises at least about 90% to 100% identity the native Phaseolus vulgaris (French bean) leghemoglobin protein of SEQ ID NO: 19. In certain embodiments, a native French bean leghemoglobin protein comprises at least about 90% to 100% identity to the native French bean leghemoglobin protein of SEQ ID NO: 19 and comprises native leghemoglobin function or activity. In certain embodiments, the WT French bean leghemoglobin gene CDS of the disclosure is abbreviated “LegPv”, wherein the WT LegPv gene CDS has been codon optimized for expression in T. reesei cells, as set forth in SEQ ID NO: 20. In other embodiments, a gene CDS encoding native French bean leghemoglobin protein encodes a leghemoglobin protein comprising at least about 40% to 100% identity the native protein of SEQ ID NO: 19.

As used herein, a “native bovine (Bos taurus) myoglobin protein” comprises at least about 80% to 100% identity the native myoglobin protein of SEQ ID NO: 3. In certain embodiments, a native myoglobin protein comprises at least about 80% to 100% identity to the native myoglobin protein of SEQ ID NO: 3 and retains (comprises) native myoglobin function or activity. In certain one or more embodiments, a gene CDS encoding native myoglobin protein encodes a myoglobin protein comprising at least about 80% to 100% identity the wild-type protein of SEQ ID NO: 3. In certain one or more embodiments, a gene CDS encoding a native myoglobin protein comprises at least about 80% to 100% identity to the native myoglobin protein of SEQ ID NO: 3 and retains (comprises) native myoglobin function or activity. In certain aspects, the WT myoglobin gene CDS is abbreviated “Mb1b”, wherein the WT Mb1b gene CDS has been codon optimized for expression in T. reesei cells, as set forth in SEQ ID NO: 4. In certain other embodiments, a gene CDS encoding native bovine myoglobin protein encodes a myoglobin protein comprising at least about 40% to 100% identity the native protein of SEQ ID NO: 3. In certain other embodiments, a gene CDS encoding native bovine myoglobin protein encodes a myoglobin protein comprising at least about 40% to 100% identity the native protein of SEQ ID NO: 3.

As used herein, phrases such as “protease inhibitor protein”, “protein protease inhibitor” or “protease inhibitor” may be used interchangeably, wherein a protease inhibitor protein may be any peptide or protein that reversibly inhibits the protease in question. According to the MEROPS database (www.ebi.ac.uk/merops/) which is depository of proteases/peptidases and the proteins that inhibit these protein/peptide hydrolytic enzymes, protease inhibitors may be classified into 38 clans and subdivided into 78 families. In certain embodiments, protease inhibitors include, but are not limited to, trypsin inhibitor proteins and subtilisin inhibitor proteins. Examples are trypsin inhibitors and subtilisin inhibitors which are known in the art, as generally described in Laskowski and Kato (1980), Strickler et al, (1992), PCT Publication No. WO1992/03529, and the like. In certain embodiments, exemplary protease inhibitors are trypsin inhibitors of Family IV and subtilisin inhibitors of Families III, VI and VII. Examples include, but are not limited to, a native Streptomyces subtilisin inhibitor (SSI) and functional variants thereof, a native Streptomyces antifibrinolytics plasminostreptin inhibitor and functional variants thereof, a native barley subtilisin inhibitor (BASI) and functional variants thereof, a native potato subtilisin inhibitor and functional variants thereof, a native tomato subtilisin inhibitor and functional variants thereof, a native eglin C inhibitor and functional variants thereof, a native Vicia faba subtilisin inhibitor and functional variants thereof, a native leupeptin inhibitor and functional variants thereof, a native soy bean trypsin inhibitor and functional variants thereof, a native pea aspartic protease inhibitor and functional variants thereof, a serine protease inhibitor such as a Bowman-Birk inhibitors (BBI) and functional variants thereof, a native serpin inhibitor and functional variants thereof, a native phytocystatin inhibitor and functional variants thereof, a native Kunitz-type inhibitor (KTI) and functional variants thereof, bi-functional α-amylase-trypsin inhibitors and functional variants thereof, mustard-type inhibitors, a potato metallo-carboxypeptidase inhibitor and functional variants thereof, a native squash inhibitor and functional variants thereof, a native and cyclotide inhibitor and functional variants thereof, and the like.

As used herein, a “gene encoding a native barley amylase subtilisin (protease) inhibitor” protein comprises at least about 90% to 100% identity to the wild-type BASI gene set forth in SEQ ID NO: 6. In certain aspects, the wild-type barley amylase subtilisin inhibitor gene is abbreviated “BASI” (italicized). In certain one or more embodiments or aspects of the disclosure, the wild-type BASI gene has been codon optimized for expression in Trichoderma strains.

As used herein, a “native barley amylase subtilisin (protease) inhibitor” protein (abbreviated herein, “BASI” protein) comprises protease inhibitor activity and at least about 90% to 100% identity to the native BASI protein of SEQ ID NO: 6. For example, one or more variant BASI proteins may be derived from the native BASI protein (SEQ ID NO 6), wherein the native or variant BASI proteins are particularly suitable for reducing/mitigating certain unwanted protease activities as described herein.

As used herein, a modified T. reesei strain named “BFZ28” was derived from the Tr1 parental strain and comprises an introduced expression cassette (Pcbh1-CBH1 core-KEX2-LegGm1b) encoding a heterologous (soybean) leghemoglobin protein.

As used herein, a modified T. reesei strain named “BGJ74” was derived from the Tr1 parental strain and comprises an introduced cassette (Pcbh1-LegGm1b) encoding a heterologous (soybean) leghemoglobin protein and an introduced cassette (Pcbh2-BASI) encoding a protease inhibitor.

As used herein, a modified T. reesei strain named “BGJ75” was derived from the Tr1 parental strain and comprises an introduced cassette (Pcbh1-Pv1b) encoding a heterologous (French bean) leghemoglobin protein and an introduced cassette (Pcbh2-BASI) encoding a protease inhibitor.

As used herein, a modified T. reesei strain named “BGJ76” was derived from the Tr1 parental strain and comprises an introduced cassette (Pcbh1-LegGm1b.Pep1ss) encoding a heterologous (soybean) leghemoglobin protein and an introduced cassette (Pcbh2-BASI) encoding a protease inhibitor.

As set forth below in TABLE 1 of the Examples section, various plasmids (vectors) have been designed, constructed, and screened for secreted expression of globin proteins in filamentous fungal strains. In particular, TABLE 1 presents the names (1^{st} column) and descriptions (2^{nd} column) of the various vectors constructed, including relevant genetic elements, such as heterologous promoter regions (3^{rd} column), signal (secretion) sequences (4^{th} column), N-terminal fusion (protein) sequences (5^{th} column), gene CDS descriptions (6^{th} column) and C-terminal fusion (protein) sequences (7^{th} column) described and exemplified herein. TABLE 1 also includes the names of the 5′ PCR primers (9^{th} column) and 3′ PCR primers (10^{th} column) used herein, which primer DNA sequences are set forth in the Sequence Listing.

As used herein, chromosomal integration cassettes for expression of leghemoglobin include cassette “HG1” (SEQ ID NO: 27; pI1-Pcbh1-LegGm1b), cassette “HG2” (SEQ ID NO: 28; pI1-Pcbh1-LegGm1b-Pep1ss), cassette “HG3” (SEQ ID NO: 29; pI1-Pcbh1-Cbh1 core-LegGm1b), cassette “HG4” (SEQ ID NO: 30; pI1-Pcbh1-Cbh1FL-LegGm1b), cassette “HG5” (SEQ ID NO: 31; pI1-Pcbh1-Cbh1-LegGm1b-His6) and cassette “HG9” (SEQ ID NO: 35; pI1c-Pcbh1-BASI-LegGm1b).

As used herein, chromosomal integration cassettes for expression of myoglobin include cassette “HG6” (SEQ ID NO: 32; pI1-Pcbh1-Cbh1FL-Mb1b) and cassette “HG7” (SEQ ID NO: 33; pI1-Pcbh1-Cbh1FL-Mb1b-His6).

As used herein, a chromosomal integration cassette for expression of a protease inhibitor (BASI) is named cassette “HG8” (SEQ ID NO: 34; pKS923-Pcbh2-BASI).

As used herein, plasmids named “pCHL852” and “pCHL853” comprise codon optimized genes encoding a French bean leghemoglobin and soybean leghemoglobin, respectively. More particularly, plasmid pCHL852 (SEQ ID NO: 47) comprises an upstream (5′) cbh1 promoter sequence operably linked to a downstream DNA sequence encoding a Cbh1 signal sequence operably linked to a downstream gene CDS (LegPv1b) encoding the leghemoglobin protein and plasmid pCHL853 (SEQ ID NO: 36) comprises an upstream (5′) cbh1 promoter sequence operably linked to a downstream DNA sequence encoding a Cbh1 (protein) signal sequence operably linked to a downstream gene CDS (LegGm1b) encoding the leghemoglobin protein.

As used herein, a plasmid named “pCHL856” comprises a codon optimized gene encoding a soybean leghemoglobin. More particularly, plasmid pCHL856 (SEQ ID NO: 37) comprises an upstream (5′) cbh1 promoter sequence (SEQ ID NO: 11) operably linked to a downstream DNA sequence encoding a Pep1 (protein) signal sequence (SEQ ID NO: 14) operably linked to a downstream gene CDS (LegGm1b) encoding the leghemoglobin protein (SEQ ID NO: 1).

As used herein, a plasmid named “pLH1088” comprises a codon optimized gene encoding a soybean leghemoglobin. More particularly, plasmid pLH1088 (SEQ ID NO: 38) comprises an upstream (5′) cbh1 promoter sequence (SEQ ID NO: 11) operably linked to a downstream DNA sequence encoding a Cbh1 signal sequence (SEQ ID NO: 13) operably linked to a downstream DNA sequence encoding a Cbh1 protein core domain (abbreviated, “Cbh1 core”; SEQ ID NO: 7) operably linked to a downstream gene CDS (LegGm1b) encoding the leghemoglobin protein (SEQ ID NO: 1).

As used herein, a plasmid named “pLH1104” comprises a codon optimized gene encoding a soybean leghemoglobin. More particularly, plasmid pLH1104 (SEQ ID NO: 39) comprises an upstream (5′) cbh1 promoter sequence (SEQ ID NO: 11) operably linked to a downstream DNA sequence encoding a Cbh1 signal sequence (SEQ ID NO: 13) operably linked to a downstream DNA sequence encoding a full-length Cbh1 protein (abbreviated, “Cbh1 FL”; (SEQ ID NO: 9) operably linked to a downstream gene CDS (LegGm1b) encoding the leghemoglobin protein (SEQ ID NO: 1).

As used herein, a plasmid named “pLH1105” comprises a codon optimized gene encoding a soybean leghemoglobin. More particularly, plasmid pLH1105 (SEQ ID NO: 40) comprises an upstream (5′) cbh1 promoter sequence (SEQ ID NO: 11) operably linked to a downstream DNA sequence encoding a Cbh1 signal sequence (SEQ ID NO: 13) operably linked to a downstream DNA sequence encoding a Cbh1 FL protein (SEQ ID NO: 9) operably linked to a downstream gene CDS (LegGm1b) encoding the leghemoglobin protein (SEQ ID NO: 1) operably linked to a downstream six-histidine (6-His) tag.

As used herein, a plasmid named “pLH1106” comprises a codon optimized gene encoding a bovine myoglobin. More particularly, plasmid pLH1106 (SEQ ID NO: 41) comprises an upstream (5′) cbh1 promoter sequence (SEQ ID NO: 11) operably linked to a downstream DNA sequence encoding a Cbh1 signal sequence (SEQ ID NO: 13) operably linked to a downstream DNA sequence encoding a Cbh1 FL protein (SEQ ID NO: 9) operably linked to a downstream gene CDS (Mb1b) encoding the myoglobin protein (SEQ ID NO: 3).

As used herein, a plasmid named “pLH1107” comprises a codon optimized gene encoding a bovine myoglobin. More particularly, plasmid pLH1107 (SEQ ID NO: 42) comprises an upstream (5′) cbh1 promoter sequence (SEQ ID NO: 11) operably linked to a downstream DNA sequence encoding a Cbh1 signal sequence (SEQ ID NO: 13) operably linked to a downstream DNA sequence encoding a Cbh1 FL protein (SEQ ID NO: 9) operably linked to a downstream gene CDS (Mb1b) encoding the myoglobin protein (SEQ ID NO: 3) operably linked to a downstream 6-Histidine tag.

As used herein, a plasmid named “pLH1108” comprises a gene encoding barley amylase subtilisin inhibitor (BASI) protein. More particularly, plasmid pLH1108 (SEQ ID NO: 43) comprises an upstream (5′) cbh2 promoter sequence (SEQ ID NO: 12) operably linked to a downstream DNA sequence encoding a Pep1 signal sequence (SEQ ID NO: 14) operably linked to a downstream DNA sequence (BASI) encoding the BASI protein (SEQ ID NO: 5).

As used herein, a plasmid named “pLH1109” comprises a codon optimized gene encoding a BASI protein. More particularly, plasmid pLH1109 (SEQ ID NO: 44) comprises an upstream (5′) cbh1 promoter sequence (SEQ ID NO: 11) operably linked to a downstream DNA sequence encoding a Pep1 signal sequence (SEQ ID NO: 14) operably linked to a downstream DNA sequence (BASI) encoding the BASI protein (SEQ ID NO: 5) operably linked to a downstream gene CDS (LegGm1b) encoding the leghemoglobin protein (SEQ ID NO: 1).

As used herein, the terms “polypeptide” and “protein” (and/or their respective plural forms) are used interchangeably to refer to polymers of any length comprising amino acid residues linked by peptide bonds. The conventional one-letter or three-letter codes for amino acid residues are used herein. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As further described below in Sections II-III and the Examples, in certain embodiments, one or more globin proteins of interest may be designed and constructed as fusion proteins. For example, in certain non-limited aspects, globin fusion proteins may comprise a N-terminus fusion (“N-fusion”) of one or more amino acid residues and/or a C-terminus fusion (“C-fusion”) of one or more amino acid residues. For instance, certain globin fusion proteins having N-term and/or C-term fusions have been designed, constructed, and described herein, as generally set forth in TABLES 1-2 of the Examples.

As used herein, the term “derivative polypeptide/protein” refers to a protein which is derived or derivable from a protein by addition of one or more amino acids to either or both the N- and C-terminal end(s), substitution of one or more amino acids at one or a number of different sites in the amino acid sequence, deletion of one or more amino acids at either or both ends of the protein or at one or more sites in the amino acid sequence, and/or insertion of one or more amino acids at one or more sites in the amino acid sequence. The preparation of a protein derivative can be achieved by modifying a DNA sequence which encodes for the native protein, transformation of that DNA sequence into a suitable host, and expression of the modified DNA sequence to form the derivative protein.

Related (and derivative) proteins include “variant proteins”. Variant proteins differ from a reference/parental protein (e.g., a wild-type protein) by substitutions, deletions, and/or insertions at a small number of amino acid residues. The number of differing amino acid residues between the variant and parental protein can be one or more, for example, 1, 2, 3, 4, 5, 6, 7, 8, 9, 10, 15, 20, 30, 40, 50, or more amino acid residues. Variant proteins can share at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a reference protein. A variant protein can also differ from a reference protein in selected motifs, domains, epitopes, conserved regions, and the like.

As used herein, the term “analogous sequence” refers to a sequence within a protein that provides similar function, tertiary structure, and/or conserved residues as the protein of interest (i.e., typically the original protein of interest). For example, in epitope regions that contain an α-helix or a β-sheet structure, the replacement amino acids in the analogous sequence preferably maintain the same specific structure. The term also refers to nucleotide sequences, as well as amino acid sequences. In some embodiments, analogous sequences are developed such that the replacement of amino acids result in a variant enzyme showing a similar or improved function. In some embodiments, the tertiary structure and/or conserved residues of the amino acids in the protein of interest are located at or near the segment or fragment of interest. Thus, where the segment or fragment of interest contains, for example, an α-helix or a 3-sheet structure, the replacement amino acids preferably maintain that specific structure.

As used herein, the term “homologous protein” refers to a protein that has similar activity and/or structure to a reference protein. It is not intended that homologues necessarily be evolutionarily related. Thus, it is intended that the term encompass the same, similar, or corresponding protein(s) (i.e., in terms of structure and function) obtained from different organisms. In some embodiments, it is desirable to identify a homologue that has a quaternary, tertiary and/or primary structure similar to the reference protein.

The degree of homology between sequences can be determined using any suitable method known in the art (e.g., programs such as GAP, BESTFIT, FASTA, and TFASTA in the Wisconsin Genetics Software Package (Genetics Computer Group, Madison, WI). As presented and described below in Section II, other globin gene/protein homologues may be identified by reference to one or more exemplary globin proteins, which globin proteins are well suited for production in one or more modified filamentous fungal strains of the disclosure.

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (1987). Useful PILEUP parameters including a default gap weight of 3.00, a default gap length weight of 0.10, and weighted end gaps. Another example of a useful algorithm is the BLAST algorithm. One particularly useful BLAST program is the WU-BLAST-2 program. Parameters “W,” “T,” and “X” determine the sensitivity and speed of the alignment. The BLAST program uses as defaults a word-length (W) of 11, the BLOSUM62 scoring matrix alignments (B) of 50, expectation (E) of 10, M′5, N′-4, and a comparison of both strands.

As used herein, the phrases “substantially similar” and “substantially identical”, in the context of at least two nucleic acids or polypeptides, typically means that a polynucleotide or polypeptide comprises a sequence that has at least about 40% to 100% sequence identity. Thus, in one or more embodiments, a substantially similar or substantially identical nucleic acid or polypeptide of the disclosure comprises at least about 40%, 50%, 60%, 70%, 80%, 90% or 100% identity to one or more sequences set forth herein. In certain related embodiments, one or more nucleic acid sequences and/or one or more protein sequences of the disclosure comprise at least about 40%, 41%, 42%, 43%, 44%, 45%, 46%, 47%, 48%, 49%, 50%, 50%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity to one or more sequences set forth herein. Sequence identity can be determined using known programs such as BLAST, ALIGN, and CLUSTAL using standard parameters. Software for performing BLAST analyses is publicly available through the National Center for Biotechnology Information. Also, databases can be searched using FASTA. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the production of the polypeptide including, but not limited to, transcription, post-transcriptional modification, translation, post-translational modification, secretion and the like.

As used herein, the terms “modification” and “genetic modification” are used interchangeably and include, but are not limited to: (a) the introduction, substitution, or removal of one or more nucleotides in a gene, or the introduction, substitution, or removal of one or more nucleotides in a regulatory element required for the transcription or translation of the gene, (b) gene disruption, (c) gene conversion, (d) gene deletion, (e) the down-regulation of a gene (e.g., antisense RNA, siRNA, miRNA, and the like), (f) specific mutagenesis (including, but not limited to, CRISPR/Cas9 based mutagenesis) and/or (g) random mutagenesis of any one or more the genes disclosed herein.

As used herein, “the introduction, substitution, or removal of one or more nucleotides in a gene encoding a protein”, such genetic modifications include the gene's coding sequence (i.e., exons) and non-coding intervening (introns) sequences.

As used herein, “disruption of a gene”, “gene disruption”, “inactivation of a gene” and “gene inactivation” are used interchangeably and refer broadly to any genetic modification that substantially disrupts/inactivates a target gene. Exemplary methods of gene disruptions include, but are not limited to, the complete or partial deletion of any portion of a gene, including a polypeptide coding sequence (CDS), a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the expression/production of the functional gene product. In certain embodiments of the disclosure, such gene disruptions prevent a host cell from expressing/producing the encoded lov gene product.

In other embodiments, a protein of interest (e.g., a globin POI) expressed/produced by the fungal cells of the disclosure may be detected, measured, assayed and the like, by protein quantification methods, gene transcription methods, mRNA translation methods and the like, including, but not limited to, protein migration/mobility (SDS-PAGE), mass spectrometry, HPLC, size exclusion, ultracentrifugation sedimentation velocity analysis, transcriptomics, proteomics, fluorescent tags, epitope tags, fluorescent protein (GFP, RFP, etc.) chimeras/hybrids and the like.

As used herein, functionally and/or structurally similar proteins are considered to be “related proteins”. Such related proteins can be derived from organisms of different genera and/or species, or even different classes of organisms (e.g., bacteria and fungi). Related proteins also encompass homologues and/or orthologues determined by primary sequence analysis, determined by secondary or tertiary structure analysis, or determined by immunological cross-reactivity.

The term “promoter” as used herein refers to a nucleic acid sequence capable of controlling the expression of a coding sequence (CDS) or functional RNA. In general, a coding sequence (CDS) is located downstream (3′) to a promoter (pro) sequence. Promoters may be derived in their entirety from a native gene or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As defined herein, the term “introducing”, as used in phrases such as “introducing into a fungal cell” at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in the cell that is to be transformed).

As used herein, “transformation” refers to introducing an exogenous DNA into a host cell so that the DNA is maintained as a chromosomal integrant or a self-replicating extra-chromosomal vector. As used herein, “transforming DNA”, “transforming sequence”, and “DNA construct” refer to DNA that is used to introduce sequences into a host cell. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein “an incoming sequence” refers to a DNA sequence that is introduced into the fungal cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, an incoming sequence is a non-functional sequence inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the fungal cell chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the fungal cell chromosome a DNA construct is integrated and directs what part of the fungal cell chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

As defined herein, a host cell “genome”, a fungal cell “genome”, or a filamentous fungus cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “vector” refers to any nucleic acid that can be replicated (propagated) in cells and can carry new genes or DNA segments (e.g., an “incoming sequence”) into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously) or can integrate into the chromosome of a host cell.

A used herein, a “transformation cassette” refers to a specific vector comprising a gene and having elements in addition to the gene that facilitate transformation of a particular host cell.

As used herein, “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” refers to a nucleic acid construct generated recombinantly or synthetically, with a series of specified nucleic acid elements that permit transcription of a particular nucleic acid in a target cell (e.g., vectors or vector elements described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing genetic modifications into the chromosome of a host cell through homologous recombination. In some embodiments, a targeting vector comprises other non-homologous sequences, e.g., added to the ends (i.e., stuffer sequences or flanking sequences). The ends can be closed such that the targeting vector forms a closed circle, such as, for example, insertion into a vector.

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, heat treatment, ammonium sulphate precipitation or other protein salt precipitation, crystallization, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “protein preparation” is any material, typically a solution, generally aqueous, comprising one or more proteins.

As used herein, the terms “broth”, “cultivation broth”, “fermentation broth” and/or “whole fermentation broth” may be used interchangeably and refer to a preparation produced by cellular fermentation that undergoes no processing steps after the fermentation is complete. For example, whole fermentation broths are typically produced when microbial cultures are grown to saturation, incubated under carbon-limiting conditions to allow protein synthesis (e.g., expression of proteins by host cells; and optionally, secretion of the proteins into cell culture medium). Typically, the whole fermentation broth is unfractionated and comprises spent cell culture medium, metabolites, extracellular polypeptides, and microbial cells.

As used herein, the phrase “treated broth” refers to broth that has been conditioned by making changes to the chemical composition and/or physical properties of the broth. Broth “conditioning” may include one or more treatments such as cell lysis, pH modification, heating, cooling, addition of chemicals (e.g., calcium, salt(s), flocculant(s), reducing agent(s), enzyme activator(s), enzyme inhibitor(s), and/or surfactant(s)), mixing, and/or timed hold (e.g., 0.5 to 200 hours) of the broth without further treatment.

As used herein, a “cell lysis” process includes any cell lysis technique known in the art, including, but not limited to, enzymatic treatments (e.g., lysozyme, proteinase K treatments), chemical means (e.g., ionic liquids), physical means (e.g., French pressing, ultrasonic), simply holding culture without feeds, and the like.

The terms “recovery”, “recovered” and “recovering” as used herein refer to at least partial separation of a protein from one or more components of a microbial broth and/or at least partial separation from one or more solvents in the broth (e.g., water or ethanol).

In certain aspects, broths in which host cells have been fermented for the production of globin proteins, with or without broth treatment, are clarified. As used herein, a “clarified” broth means a broth which has been subjected to at least one clarification process to remove cell debris and/or other insoluble components. Clarification processes, as understood in the art include, but are not limited to, centrifugation techniques, cross-flow membrane filtration techniques, solid/liquid filtration techniques, and the like.

“Cell debris” refers to cell walls and other insoluble components that are released or formed after disruption of the cell membrane (e.g., after performing a cell lysis process).

In certain aspects, separation of solvents, as understood in the art include, but are not limited to ultrafiltration, evaporation, spray drying, freezer drying. The obtained solution is referred to as “clarified broth concentrate”, “UF concentrate”, or “ultrafiltrate concentrate”.

As used herein, the term “cell mass” refers to the cell component (including intact and lysed cells) present in a liquid (submerged) culture. Cell mass can be expressed in dry cell weight (DCW) or wet cell weight (WCW).

### II. Recombinant Filamentous Fungal Cells Producing Heterologous Globins

As briefly set forth above, despite current knowledge related to globin proteins (e.g., leghemoglobins, cyanoglobins, hemoglobins, myoglobins, leghemoglobins, etc.), the isolation and/or production globin proteins and their various uses and applications, there remain ongoing and unmet needs in the art. For instance, due to growing demands for so-called “non-animal” derived meat substitutes, certain leguminous (plant) derived globins (i.e., leghemoglobins) have been contemplated for use. In certain examples, isolation of leghemoglobins for use as a meat-substitute have been described, wherein the leghemoglobin proteins were isolated and purified from plant (legume) sources (PCT Publication No. WO2013/010042). In other instances, the expression of leghemoglobin proteins in yeast cells (e.g., S. cerevisiae; P. pastoris) have been attempted, which generally require extensive genetic modifications of the host cell to co-express the entire heme biosynthesis pathway and the leghemoglobin protein (PCT Publication No. WO2016/183163).

As described in Krainer et al. (2015), insufficient incorporation of heme is considered a central impeding cause in the recombinant production of active heme proteins. In particular, Krainer et al. (2015) investigated the effects of both pathway engineering and medium supplementation to optimize the recombinant production of the heme protein horseradish peroxidase (HRP) in methylotrophic yeast (P. pastoris). As summarized in Krainer et al. (2015), in contrast to studies with other host organisms, (a) co-overexpression of genes of the endogenous heme biosynthesis pathway in P. pastoris did not improve the recombinant production of active heme (HRP) enzyme, (b) medium supplementation with the commonly used precursor 5-aminolevulonic acid (ALA) did not affect yield of the active heme (HRP) enzyme, whereas (c) medium supplementation with hemin increased the yield of active heme (HRP) enzyme, concluding that the yield of active peroxidase enzyme from P. pastoris can be easily enhanced by supplementation of the cultivation medium with hemin.

More recently, Shao et al. (2022) have described a P. pastoris strain capable of high-yield secretory production of functional leghemoglobin, which was developed through gene dosage optimization and heme pathway consolidation. In particular, the heme biosynthetic pathway was engineered by increasing the heterologous leghemoglobin copy numbers and consolidating the native heme biosynthesis pathway to address challenges in heme depletion and leghemoglobin secretion, wherein such P. pastoris strain engineering strategies increased secretion of leghemoglobin without the need to supplement the media with expensive precursors (e.g., hemin; Shao et al., 2022).

In other instances, non-animal derived meat-like materials/ingredients obtained from genetically modified cyanobacteria (blue-green algae) comprising polynucleotides encoding heterologous heme-containing proteins (e.g., leghemoglobin, cyanoglobin) have been described (PCT Publication WO2019/079135). As generally set forth in the WO2019/079135 publication, certain genetic modifications of the cyanobacterial host strain were required to improve the levels of the globin proteins, such as genetic modifications reducing (lowering) the level of heme oxygenase in the host strain, genetic modifications that knockdown the gene encoding magnesium chelatase (MgCh) in the host strain, genetic modifications that overexpress ferrochelatase (FeCh) in the host strain, genetic modifications that knockdown the level of gun4 (MgCh activator) in the host strain and the like.

In certain other instances, the heme biosynthesis pathway of an Aspergillus niger strain was evaluated in the context of producing lignin degrading peroxidases (i.e., heme containing class II peroxidases; Franken et al., 2011). As summarized by Franken et al. (2011), cofactor availability and incorporation has been shown to be a limiting factor in the production of fungal peroxidases in A. niger, which require heme as a cofactor. In particular, Franken et al. (2011) states that peroxidase production can be increased by the supplementation of hemoglobin or hemin to the fermentation medium, but the mechanism behind heme uptake is poorly understood and the approach is too costly to be suited for industrial purposes.

Thus, as generally described above, it is evident that there remain ongoing uncertainties and discrepancies in the art as related to, inter alia, identifying optimal host cell genetic modifications necessary for the enhanced production of globin proteins, the need or requirement for media (broth) supplementations (e.g., hemin, ALA) for the enhanced production of globin proteins, and the like. For instance, as demonstrated in certain yeast strains (e.g., S. cerevisiae; P. pastoris), depending upon the strain of yeast screened and the particular globin protein expressed thereby, the need or requirement for the up-regulation (e.g., over-expression) and/or down-regulation of one or more heme pathway biosynthesis genes varies. Likewise, in other aspects, depending upon the strain of yeast being fermented vis-à-vis the globin protein expressed thereby, the need or requirement for media supplementations can vary significantly. As reviewed in Franken et al. (2011), the use of an Aspergillus sp. fungal strain for production of lignin degrading peroxidases requires media supplementation (e.g., hemin), suggesting that a more thorough understanding of the heme biosynthesis pathway and its regulation in A. niger are required for further strain improvements, most desirably without the need for media supplementation.

Thus, it generally remains unknown if filamentous fungal strains such as Aspergillus can produce (heme containing) globin proteins at sufficiently high levels desired in the art. In particular, the design, construction, identification, cultivation/fermentation, and the like of genetically modified (recombinant) host organisms comprising enhanced globin production phenotypes (e.g., protein titers, specific productivity, protein yields, volumetric productivity, carbon conversion efficiency, etc.) and methods thereof are important economic factors of globin protein production costs, particularly under large-scale (industrial) fermentation conditions.

Based on the foregoing, Applicant has surprisingly observed that recombinant filamentous fungal cells are particularly well-suited for large scale production of heterologous globin proteins. More specifically, as set forth in the Examples section below, Applicant designed, constructed, evaluated and the like recombinant polynucleotides (e.g., expression cassettes) encoding heterologous globin proteins, wherein the cassettes were introduced into filamentous fungal cells for the expression and secretion of the globin proteins. In particular, as set forth in Example 1, Applicant constructed vectors for the expression of a soybean leghemoglobin (SEQ ID NO: 1) as a secreted protein (Example 1A), the expression of a French bean leghemoglobin (SEQ ID NO: 19) as a secreted protein (Example 1B), and the expression of the soybean leghemoglobin (SEQ ID NO: 1) as a secreted fusion-protein (Example 1C). As presented and described in Example 2, Applicant further constructed vectors for the expression of a heterologous bovine myoglobin protein (SEQ ID NO: 3) in filamentous fungal cells. For example, the various nucleic acid (DNA) sequences and genetic elements used for the construction of one or more modified (recombinant) filamentous fungal strains of the disclosure are set forth below in TABLE 1 of the Examples section. Likewise, certain chromosomal integration vectors for the expression of leghemoglobin, myoglobin, and a protease inhibitor protein (BASI; SEQ ID NO: 5) are set forth in TABLE 2 of the Examples section.

As set forth in Example 3, Applicant further designed and constructed recombinant filamentous fungal cells via targeted integration of one or more expression cassettes. In particular, leghemoglobin and myoglobin expressing filamentous fungal strains were generated by Cas9 guided targeted integration into the genome (Example 3). In addition, to assess possible proteolytic degradation of the secreted globin proteins (e.g., hydrolytic degradation via native proteases secreted by the host strain), an expression cassette encoding an exemplary secreted protease inhibitor protein (BASI) was integrated into certain strains for the co-expression of a globin protein (e.g., leghemoglobin) and a protease inhibitor (e.g., BASI).

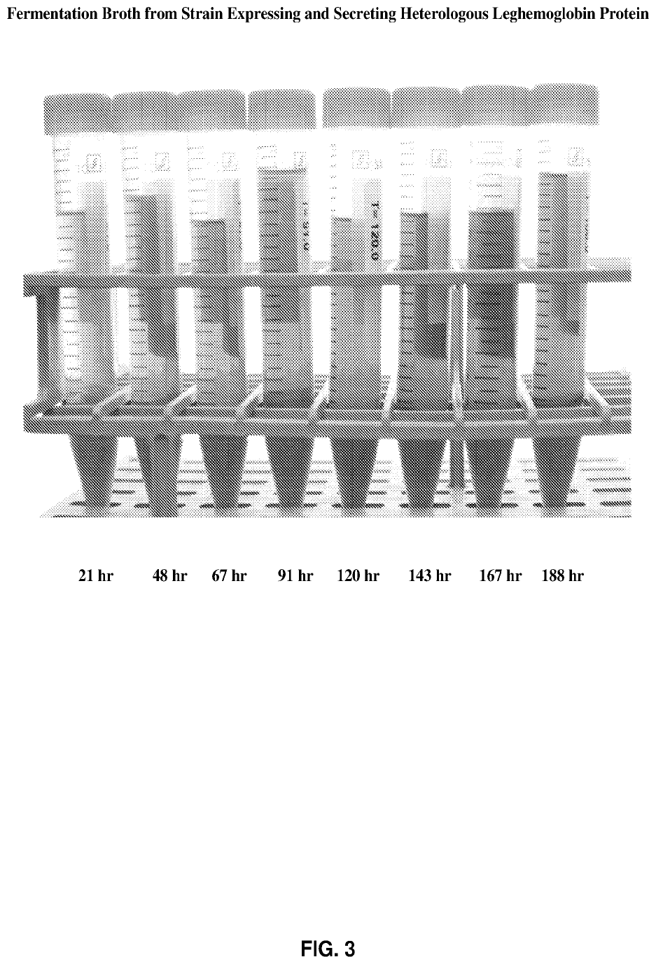

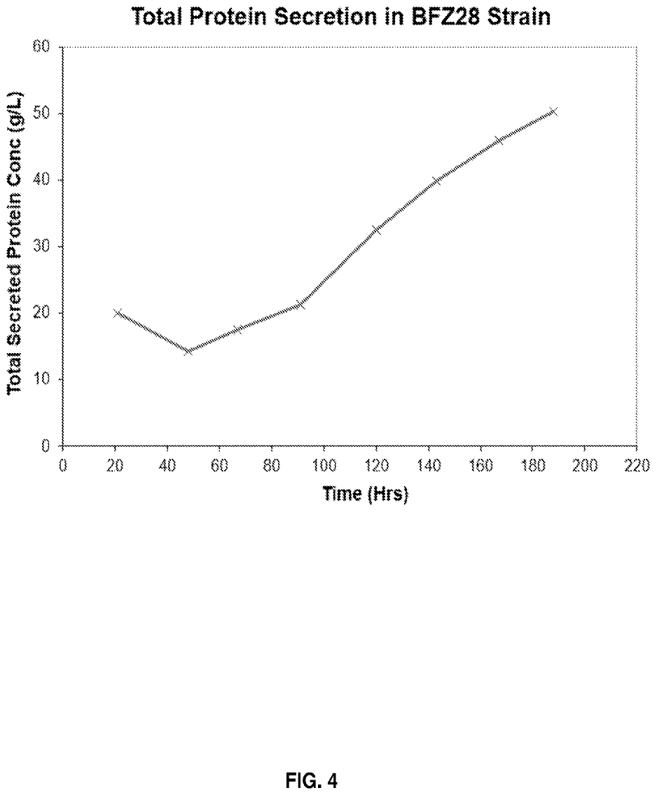

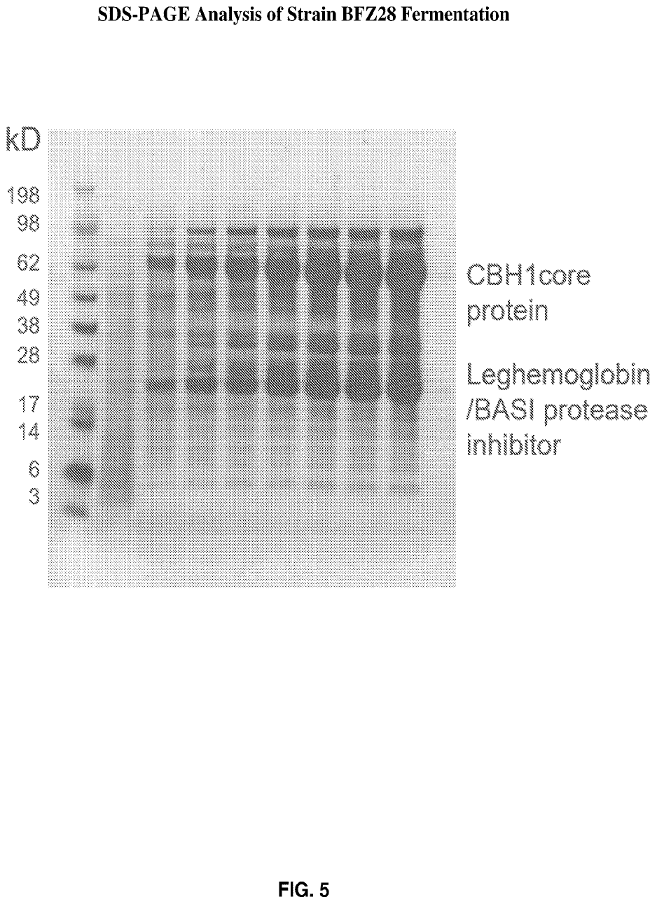

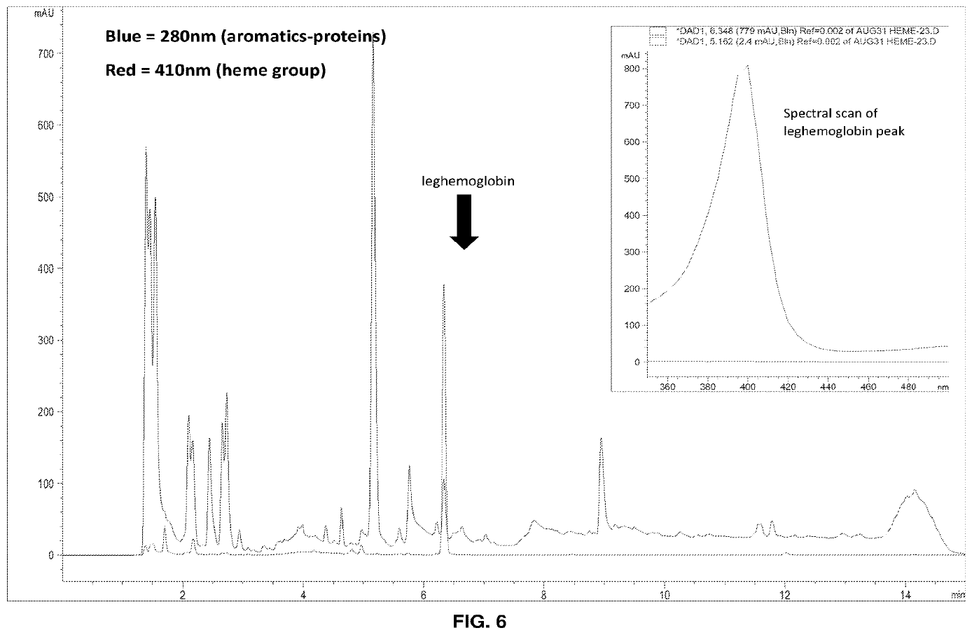

As generally set forth and described in Example 4, a fed-batch fermentation was performed in a two-liter (2 L) bioreactor for the filamentous fungal strain BFZ28 (Pcbh1-CBH1 core-KEX2-LegGm1b), wherein ten milliliter (10 mL) whole broth samples were taken every twenty-four (24) hours and frozen at −20° C. More particularly, as shown in FIG. 3, the red (dark) color formation in the supernatant indicates secretion of the globin proteins into the culture supernatants. In particular, the total protein secretion of the BFZ28 is shown in FIG. 4, wherein the total protein concentration after about 188 hours fermentation is approximately 50 grams/liter (50 g/L). As shown in FIG. 5, supernatants from strain BFZ28 were analyzed by SDS-PAGE, wherein the upper protein band of approximately (~) 50 kDa is the Cbh1 core protein, the leghemoglobin band (~16 KDa) is either co-migrating with the BASI protease inhibitor at ~20 kDa, or present as a minor band −17 kDa. In addition, to verify/confirm expression of leghemoglobin, the fermentation supernatant was analyzed by HPLC analysis of the 188-hour supernatant sample from the BFZ28 fermentation run. As shown in FIG. 6, the porphyrin (heme) prosthetic group of the leghemoglobin protein is detected at wavelength of 410 nm, which co-migrated with a protein peak detected at 280 nm with a 6.4-minute retention time. The inset drawing of FIG. 6 shows the spectral scan of the leghemoglobin peak at 6.4-minutes retention time, confirming the peak absorbance of the leghemoglobin protein at 410 nm.

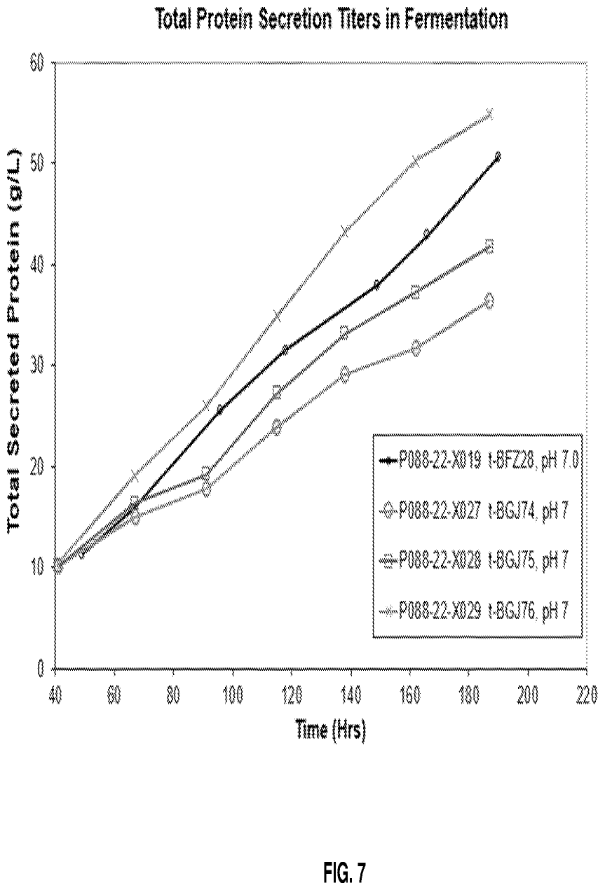

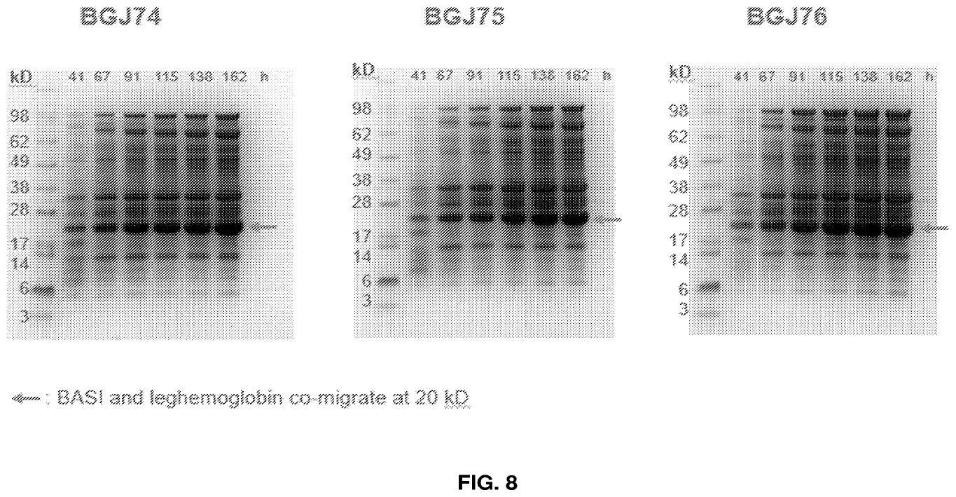

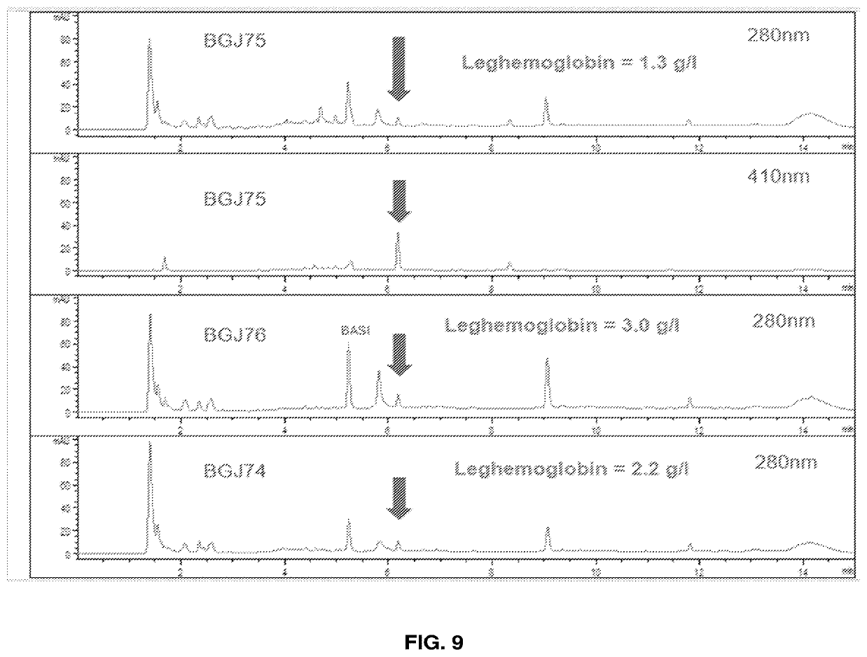

As briefly set forth above, to assess the role of proteolytic degradation on the secreted globin proteins, an expression cassette encoding an exemplary secreted protease inhibitor (BASI) was integrated into certain strains for the co-expression of the globin protein with the (BASI) protease inhibitor. More particularly, modified strains “BGJ74” (Pcbh1-LegGm1b, Pcbh2-BASI), “BGJ75” (Pcbh1-Pv1b, Pcbh2-BASI) and “BGJ76” (Pcbh1-LegGm1b.Pep1ss, Pcbh2-BASI) were constructed and fermented as generally described in Example 5. For instance, as presented in FIG. 7, the total soluble proteins secreted in the fermentation runs were plotted versus the effective fermentation time (EFT, hours), together with the data from the BFZ28 fermentation run (Example 4). As shown in FIG. 8, the fermentation supernatants were analyzed via SDS-PAGE, wherein the presence of the (heme-containing) leghemoglobin was confirmed via HPLC as shown in FIG. 9. As described in Example 5, the total protein secretion titers at the end of the 188-hour fermentation run are presented TABLE 4, with leghemoglobin titers (g/L) calculated based on the protein peak areas at 280 nm. For example, recombinant filamentous fungal strains expressing the soybean leghemoglobin (BFZ28, BGJ74, BGJ76) and the French bean leghemoglobin (BGJ75) were capable of secreting the leghemoglobin at high titers (g/L) during fermentation, as shown in TABLE 4.

Thus, certain one or more embodiments of the disclosure are related to recombinant (modified) filamentous fungal cells capable of producing heterologous globin proteins for use in food materials, food ingredients, flavor modifiers, aroma modifiers and the like. In certain embodiments, globin proteins are selected from the group consisting of leghemoglobins, myoglobins, hemoglobins, cyanoglobins and non-symbiotic hemoglobins. In certain other one or more embodiments, globin proteins include the group consisting of leghemoglobins, hemoglobins, non-symbiotic hemoglobins, myoglobins, neuroglobins, cytoglobins, protoglobins, truncated 2/2 globins, HbN, cyanoglobin, HbO, Glb3, and Hell's gate globins, bacterial hemoglobins and ciliate myoglobins. For example, as generally known in the art, members of the globin-like superfamily include a wide variety of all-helical proteins that bind porphyrins and play various roles in all three kingdoms of life, including sensors or transporters of oxygen. The globin-like superfamily includes M/myoglobin-like, S/sensor globin, and T/truncated globin (TrHb) families, and the phycobiliproteins (PBPs).

In certain other one or more embodiments, the disclosure therefore provides methods for identifying and obtaining suitable globin (DNA/protein) sequences for expression in one or more modified filamentous fungal cells described herein. In certain aspects, one or more suitable leghemoglobin sequences can be identified and obtained from a variety of plant sources, such as various legume species and their varieties, including but not limited to, soybeans, fava beans, lima bean, cowpeas, English peas, yellow peas, lupines, kidney beans, garbanzo beans, peanuts, alfalfa, vetch hay, clover, lespedeza, pinto beans and the like. In certain embodiments, a leghemoglobin protein comprises at least about 90% identity to a leghemoglobin protein derived from a plant selected from the group consisting of soybeans, fava beans, lima beans, cowpeas, English peas, French beans, yellow peas, lupines, kidney beans, garbanzo beans, peanuts, alfalfas, vetch hays, clovers, lespedezas, and pinto beans. In certain other one or more embodiments, the leghemoglobin protein comprises at least about 90% identity to the soybean leghemoglobin protein SEQ ID NO: 1. In certain other one or more embodiments, the leghemoglobin protein comprises at least about 90% identity to the French bean leghemoglobin protein SEQ ID NO: 19. In other one or more embodiments, a myoglobin protein comprises at least about 90% identity to SEQ ID NO: 3. In other one or more embodiments, recombinant filamentous fungal cells of the disclosure comprise an introduced polynucleotide (expression cassette) encoding a protease inhibitor protein.

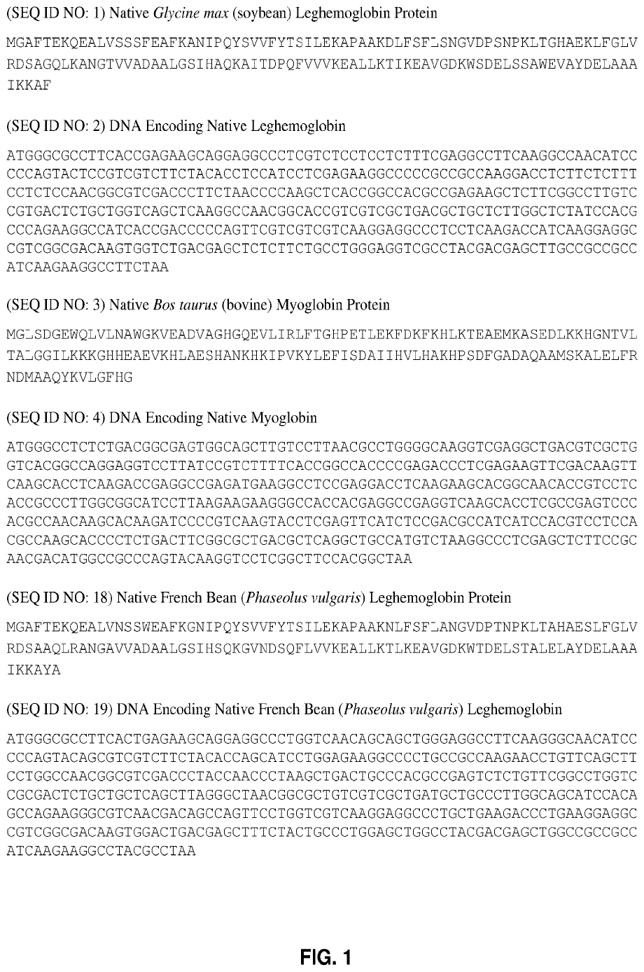

For example, the native soybean leghemoglobin protein (SEQ ID NO: 1; FIG. 1) comprises 145 amino acid residues, wherein amino acid positions 8-111 comprise a globin-like superfamily domain. In addition, the native soybean leghemoglobin protein (FIG. 1) comprises a class 1-2 nonsymbiotic hemoglobin domain at residue positions 4-144 of SEQ ID NO: 1. The leghemoglobin protein heme binding sites include amino acid positions L44, F45, S46, F47, K58, H62, K65, L66, F67, L69, V70, A88, L89, I92, H93, K96, 198, Q102, F103, Y134, L137, A138 and I141 (FIG. 1; SEQ ID NO: 1, shown as bold residues).

Thus, in certain embodiments, one or more leghemoglobin protein sequences may be derived from organisms including, but not limited to, Abrus precatorius (Accession No. XP_027365674.1), Astragalus canadensis (Accession No. QAX32739.1), Astragalus sinicus (Accession No. ABB13622.1), Cajanus cajan (Accession No. XP_020222796.1), Canavalia lineata (Accession No. P42511.1), Cicer arietinum (Accession No. XP_004490880.1), Galega orientalis (Accession No. QAX32752.1), Glycine soja Accession No. KAG4983849.1), Glycyrrhiza uralensis (Accession No. QAX32708.1), Lotus japonicus (Accession No. AFK42883.1), Medicago sativa (Accession No. AAA32657.1), Medicago truncatula (Accession No. XP_003616494.1), Mucuna pruriens (Accession No. RDX62803.1), Onobrychis viciifolia (Accession No. QAX32720.1), Ononis spinosa (Accession No. QAX32757.1), Phaseolus vulgaris (Accession No. XP_007144265.1), Pisum sativum (Accession No. XP_050917485.1), Psophocarpus tetragonolobus (Accession No. P27199.1), Sesbania rostrata (Accession No. P14848.2), Trifolium pratense (Accession No. XP_045786945.1), Trifolium subterraneum (Accession No. GAU42435.1), Vicia faba (Accession No. P93849.3), Vigna angularis (Accession No. XP_017409386.1), Vigna radiata var. radiata (Accession No. XP_014513332.1), Vigna umbellate (Accession No. XP_047166207.1) and Vigna unguiculata (Accession No. NP_001363034.1). In certain aspects, leghemoglobin proteins will comprise a nearly identical absorbance spectrum and visual appearance to myoglobin proteins derived from animal muscle.

As presented in FIG. 1, the native bovine myoglobin protein (SEQ ID NO: 3) comprises 154 amino acid residues, wherein amino acid positions 7-112 comprise a globin-like superfamily domain. Thus, in certain embodiments, one or more myoglobin protein sequences may be derived from organisms including, but not limited to, Ailuropoda melanoleuca (Accession No. XP_002925619.1), Balaena mysticetus (Accession No. R9RZK8.1), Balaenoptera acutorostrata scammony (Accession No. XP_007165766.1), Balaenoptera musculus (Accession No. XP_036722853.1), Bos mutus (Accession No. MXQ80090.1), Bos taurus (Accession No. NP_776306.1), Bubalus bubalis (Accession No. XP_006074486.1), Camelus ferus (Accession No. XP_006183931.1), Capra hircus (Accession No. XP_005680660.1), Ceratotherium simum simum (Accession No. XP_004418145.1), Cervus elaphus (Accession No. P02191.2), Crocuta (Accession No. KAF0881531.1), Delphinapterus leucas (Accession No. XP_022455612.1), Delphinus capensis (Accession No. AMN15044.1), Enhydra lutris kenyoni (Accession No. XP_022374654.1), Equus asinus (Accession No. XP_014688187.1), Equus caballus (Accession No. NP_001157488.1), Globicephala melas (Accession No. XP_030711253.1), Gorilla gorilla (Accession No. XP_018874109.1), Gulo gulo (Accession No. VCW91177.1), Hexaprotodon liberiensis (Accession No. AGM75766.1), Hyaena hyaena (Accession No. XP_039086409.1), Hyperoodon ampullatus (Accession No. AGM75769.1), Indopacetus pacificus (Accession No. Q0KIY9.3), Inia geoffrensis (Accession No. P02181.2), Lagenorhynchus obliquidens (Accession No. XP_026964057.1), Lemur catta (Accession No. XP_045408951.1), Lepilemur mustelinush (Accession No. P02169.2), Lipotes vexillifer (Accession No. XP_007456317.1), Lontra canadensis (Accession No. XP_032692617.1), Lutra lutra (Accession No. P11343.3), Manis pentadactyla (Accession No. XP_036747296.1), Megaptera novaeangliae (Accession No. P02178.2), Meles meles (Accession No. XP_045868724.1), Mesoplodon carlhubbsi (Accession No. P02183.2), Microcebus murinus (Accession No. XP_012642551.1), Monodon monoceros (Accession No. XP_029061405.1), Neophocaena asiaeorientalis (Accession No. XP_024599230.1), Orcinus orca (Accession No. XP_004286254.1), Oryctolagus cuniculus (Accession No. XP_008255312.3), Ovis aries (Accession No. NP_001072126.1), Pan paniscus (Accession No. XP_008973239.1), Phacochoerus africanus (Accession No. XP_047642382.1), Phoca vitulina (Accession No. XP_032272566.1), Physeter catodon (Accession No. 5YCG_A), Procyon lotor (Accession No. AGM75763.1), Propithecus coquereli (Accession No. XP_012500179.1), Stenella attenuate (Accession No. QOKIY6.3), Suricata suricatta (Accession No. XP_029811662.1), Sus scrofa (Accession No. NP_999401.1), Tupaia chinensis (Accession No. XP_006170382.1), Tursiops truncatus (Accession No. AMN15042.1), Ursus maritimus (Accession No. NP_001288305.1) and Ziphius cavirostris (Accession No. P02182.2).

As described in Section III below, in other one or more embodiments, recombinant filamentous fungal cells of the disclosure comprise an introduced polynucleotide (expression cassette) encoding a protease inhibitor protein. In certain other one or more embodiments, expression cassettes include or comprise nucleic acid (DNA) sequence(s) encoding fusion proteins, such as upstream (5′) DNA fused sequences (N-terminal fusions) and/or downstream (3′) DNA fused sequences (C-terminal fusions), and the like. In other embodiments, one or more cassettes are integrated into the genome of the cell. In certain other embodiments, recombinant filamentous fungal cells comprise at least two introduced expression cassettes encoding the same or different globin proteins.

In certain other embodiments, recombinant filamentous fungal cells comprise deletions or disruptions of one or more endogenous genes encoding one or more secreted proteases. In certain embodiments, deletions or disruptions of endogenous genes encoding one or more secreted proteases include, but are not limited to, subtilisin-like serine proteases, aspartic proteases, trypsin-like serine proteases, glutamic proteases, and aminopeptidases.

In yet other embodiments, recombinant filamentous fungal cells of the disclosure comprise one or more deletions of highly expressed endogenous genes. For example, in the case of T. reesei cells, highly expressed endogenous genes include, but are not limited to, one or more secreted lignocellulosic degrading enzymes (e.g., cellobiohydrolases, xylanases, endoglucanases, β-glucosidases). Thus, in certain embodiments, filamentous fungal cells of the disclosure are genetically modified to be deficient in the production of one or more highly expressed lignocellulosic degrading enzymes. For instance, in T. reesei cells, suitable genetic modifications include rendering the cell deficient in the production of one or more endogenous genes selected from the cellobiohydrolase 1 (cbh1) gene, the cellobiohydrolase 2 (cbh2) gene, the endoglucanase 1 (egl1) gene and the endoglucanase 2 (egl2) gene.

### III. Polynucleotide Constructs and Molecular Biology

As set forth above, certain embodiments of the disclosure are related to modified filamentous fungal strains comprising enhanced globin protein productivity phenotypes (e.g., protein titers, specific productivity, protein yields, volumetric productivity, carbon conversion efficiency, etc.). Thus, certain embodiments of the disclosure are related to, inter alia, molecular biology, genetic modifications, polynucleotides, genes, gene coding sequences (CDS), ORFs, vectors, expression cassettes, fusion proteins, protein linker sequences, cleavable protein linker sequences and the like. In certain embodiments, the disclosure provides recombinant nucleic acids (polynucleotides) comprising a gene or gene CDS encoding a globin protein. In particular, certain embodiments provide polynucleotide constructs (e.g., expression cassettes) encoding globin proteins for the expression and secretion of the globin protein into the media/fermentation broth. In certain aspects, one or more expression cassettes for the secretion of a globin protein may be generically presented by one or more schematics.

For example, an expression cassette encoding a secreted globin protein may be presented schematically as: 5′-[pro]-[sig-seq]-[globin CDS]-3′; wherein the expression cassette comprises (in the 5′ to 3′ direction) a promoter (pro) region sequence operably linked to a nucleic acid (sig-seq) encoding a protein (signal) secretion sequence operably linked to a nucleic acid (globin CDS) encoding the globin protein.

In certain embodiments, the promoter (pro) region sequence is a strong promoter functional in the host filamentous fungal strain. As an example, such strong promoters (pro) region sequences functional in Trichoderma sp. filamentous fungal cells include, but are not limited to, the T. reesei CBH1 promoter, CBH2 promoter, Xyn3 promoter, Gla1 promoter, Egl2 promoter, etc. In certain embodiments, a strong promoter may be referred to as a promoter over-expressing a globin protein.

Although certain protein (signal) secretion sequences are exemplified herein (e.g., Cbh1 secretion sequence (SEQ ID NO: 13), Pep1 secretion sequence (SEQ ID NO: 14), one of skill in the art may screen, identify, and select other suitable protein signal/secretion sequences functional in filamentous fungal cells. For example, in certain aspects, globin protein secretion in one or more filamentous fungal cells of the disclosure can be identified using one or more signal (secretion) peptide sequences from highly secreted filamentous fungal proteins known in the art. In certain other embodiments, globin protein secretion in one or more filamentous fungal cells may be screened and identified by reference to one or more native protein secretion/signal sequences and functional variants thereof, including, but not limited to, a Talaromyces sp. β-mannanase secretion sequence, a Talaromyces sp. glucoamylase secretion sequence, a Trichoderma sp. Cbh2 secretion sequence, a Trichoderma sp. glucoamylase secretion sequence, a Humicola sp. Cel45 secretion sequence, a Neurospora sp. chitin synthase secretion sequence, an Aspergillus sp. α-galactosidase (GlaA) secretion sequence, an Aspergillus sp. PepN secretion sequence, a Trichoderma harzianum aspartyl protease (PapA) secretion sequence, a Myceliophthora sp. IMI 387099 Xylanase secretion Sequence, and the like.

As further presented in the Examples, one or more expression cassettes encoding a secreted globin protein may include or comprise nucleic acid (DNA) sequence(s) encoding fusion proteins, protein linker sequences, cleavable (protein) linker sequences and the like. For example, an expression cassette encoding a secreted globin protein having a N-terminus fusion (N-fusion) may be presented schematically as: 5′-[pro]-[sig-seq]-[N-fusion]-[globin CDS]-3′; wherein the cassette comprises (in the 5′ to 3′ direction) a promoter (pro) region operably linked to a nucleic acid (sig-seq) encoding a protein (signal) secretion sequence operably linked to a nucleic acid (N-fusion) encoding the N-terminal fusion protein operably linked to a nucleic acid (globin CDS) encoding the globin protein. Likewise, an expression cassette encoding a secreted globin protein having a C-terminus fusion (C-fusion) may be presented schematically as: 5′-[pro]-[sig-seq]-[globin CDS]-[C-fusion]-3′; wherein the expression cassette comprises (in the 5′ to 3′ direction) a promoter (pro) region sequence operably linked to a nucleic acid (sig-seq) encoding a protein (signal) secretion sequence operably linked to a nucleic acid (globin CDS) encoding a globin protein operably linked to a nucleic acid (C-fusion) encoding the C-terminal fusion protein.

Thus, in certain other embodiments, an expression cassette encoding a secreted globin protein having a N-terminus fusion (N-fusion) and a C-terminus fusion (C-fusion) may be presented schematically as: 5′-[pro]-[sig-seq]-[N-fusion]-[globin CDS]-[C-fusion]-3′; wherein the expression cassette comprises (in the 5′ to 3′ direction) a promoter (pro) region sequence operably linked to a nucleic acid (sig-seq) encoding a protein (signal) secretion sequence operably linked to a nucleic acid (N-fusion) encoding the N-terminal fusion protein operably linked to a nucleic acid (globin CDS) encoding a globin protein operably linked to a nucleic acid (C-fusion) encoding the C-terminal fusion protein.

For instance, as shown in TABLES 1-2 (Example 2), the T. reesei Cbh1 protein is an exemplary N-terminal fusion protein, wherein the nucleic acid (leghemoglobin CDS) encoding the leghemoglobin protein comprises a nucleic acid (Cbh1) encoding the Cbh1 protein positioned upstream (5′) and operably linked to the nucleic acid encoding the leghemoglobin protein (e.g., 5′-[pro]-[sig-seq]-[Cbh1]-[leghemoglobin CDS]-3′). In other embodiments, a six-histidine amino acid (6-His) tag is an exemplary C-terminal fusion protein (peptide), wherein the nucleic acid (leghemoglobin CDS) encoding the leghemoglobin protein comprises a nucleic acid (6-His) encoding the 6-His peptide positioned downstream (3′) and operably linked to the nucleic acid encoding the leghemoglobin protein (e.g., 5′-[pro]-[sig-seq]-[leghemoglobin CDS]-[6-His]-3′). In certain other embodiments, the Cbh1 protein and 6-His peptide are exemplary N-terminal and C-terminal proteins fused the leghemoglobin protein (e.g., 5′-[pro]-[sig-seq]-[Cbh1]-[leghemoglobin CDS]-[6-His]-3′).

In certain other embodiments, one or more cassettes comprise one or more upstream (N-linkers) and/or downstream (C-linkers) nucleic acids encoding one or more (protein/peptide/amino acid) linker amino acid sequences. In certain embodiments, a protein/peptide/amino acid linker sequence is a cleavable sequence (e.g., a KEX2 cleavage site). As described in Example 4, strain BFZ28 comprises an introduced cassette (Pcbh1-CBH1 core-KEX2-LegGm1b) encoding a Cbh1-lectin fusion protein, wherein the nucleic acid (KEX2) encoding the cleavable KEX2 linker is placed between the nucleic acid (Cbh1) encoding the Cbh1 protein and the nucleic acid (leghemoglobin CDS) encoding the leghemoglobin protein (e.g., 5′-[pro]-[sig-seq]-[Cbh1]-[KEX2]-[leghemoglobin CDS]-3′). For example, during protein secretion in a fungal cell, certain proteins are cleaved by KEX2, a member of the KEX2 or “kexin’ family of serine peptidase (EC 3.4.21.61). As described in US Patent Publication No. US2014/0024067 and U.S. Pat. No. 8,936,917 (each incorporated herein by reference in its entirety), KEX2 is a highly specific calcium-dependent endopeptidase that cleaves the peptide bond immediately C-terminal to a pair of basic amino acids (the “KEX2 site”) in a protein substrate (e.g., globin) during secretion of that (globin) protein. For example, KEX2 (cleavage) sites may be included in the construction of expression cassettes encoding one or more globin proteins, as generally described in US Patent Publication No. US2014/0024067. Likewise, U.S. Pat. No. 8,936,917 describes a modified KEX2 cleavage site with a pre-sequence (VAVE) that improves the cleavage efficiency at the KEX2 site following the pre-sequence. Although the KEX2 cleavage site has been exemplified, other protease cleavage sites functional in filamentous fungal cells can be used for the cleavage of a peptide linker between a protein fusion partner and the globin protein. Examples of additional protease cleavable linkers include, but are not limited to, STE13 described in La Maquer et al. (2019) and the self-cleaving 2A peptide described in Subramanian et al. (2017).

In other embodiments, one or more cassettes encoding a globin protein comprise a terminator region sequence (term) operably linked and positioned at the 3′ end.

In certain embodiments, a polynucleotide of the disclosure may comprise one or more selectable markers. Selectable markers for use in filamentous fungi include, but are not limited to, als1, amdS, hygR, pyr2, pyr4, pyrG, sucA, a bleomycin resistance marker, a blasticidin resistance marker, a pyrithiamine resistance marker, a chlorimuron ethyl resistance marker, a neomycin resistance marker, an adenine pathway gene, a tryptophan pathway gene, a thymidine kinase marker and the like. In a particular embodiment, the selectable marker is pyr2, which compositions and methods of use are generally set forth in PCT Publication No. WO2011/153449.

Standard techniques for transformation of filamentous fungi and culturing the fungi (which are well known to one skilled in the art) are used to transform a fungal host cell of the disclosure. Thus, the introduction of a DNA construct or vector into a fungal host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated micro-projectiles, gene gun or biolistic transformation, protoplast fusion and the like. General transformation techniques are known in the art.

Often, transformation of Trichoderma sp. fungal cells uses protoplasts or cells that have been subjected to a permeability treatment, typically at a density of 10⁵ to 10⁷ per mL, particularly about 2×10⁶/mL. A volume of 100 μL of these protoplasts or cells in an appropriate solution (e.g., 1.2 M sorbitol and 50 mM CaCl₂) is mixed with the desired DNA. Generally, a high concentration of polyethylene glycol (PEG) is added to the uptake solution. Additives, such as dimethyl sulfoxide, heparin, spermidine, potassium chloride and the like, may also be added to the uptake solution to facilitate transformation. Similar procedures are available for other fungal host cells (e.g., see U.S. Pat. Nos. 6,022,725 and 6,268,328, both of which are incorporated by reference).

Thus, the methods and compositions of instant disclosure generally rely on routine techniques in the field of recombinant genetics. For example, in certain embodiments, a gene encoding a heterologous globin protein of interest is introduced into a filamentous fungal (host) cell. In certain embodiments, the gene (or gene CDS) is cloned into an intermediate vector, before being transformed into a filamentous fungal (host) cell for replication and/or expression. These intermediate vectors can be prokaryotic vectors, such as, e.g., plasmids, or shuttle vectors. In certain embodiments, the expression of the gene or gene CDS encoding the globin protein is under the control of a heterologous promoter, which can be a heterologous constitutive promoter or a heterologous inducible promoter, particularly a strong promoter capable of over-expressing the globin protein.

The expression vector typically contains a transcription unit or “expression cassette” that contains all the additional elements required for the expression of the heterologous sequence. For example, a typical expression cassette contains an upstream (5′) promoter operably linked to a nucleic acid sequence encoding a protein of interest and may further comprise nucleic acid sequences encoding protein (signal) secretion sequences, nucleic acid sequences required for efficient polyadenylation of the transcript, ribosome binding sites, and translation termination sequences. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes. Although any fungal terminator is likely to be functional in the present invention, preferred terminators include: the terminator from Trichoderma cbh1 gene, the terminator from Aspergillus nidulans trpC gene and the Aspergillus awamori or Aspergillus niger glucoamylase genes.

The particular expression vector used to transport the genetic information into the cell is not particularly critical. Any of the conventional vectors used for expression in eukaryotic or prokaryotic cells may be used. Standard bacterial expression vectors include bacteriophages X and M13, as well as plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences. The particular antibiotic resistance gene chosen is not dispositive either, as any of the many resistance genes known in the art may be suitable. The prokaryotic sequences are preferably chosen such that they do not interfere with the replication or integration of the DNA in the filamentous fungal host.

The methods of transformation of the present invention may result in the stable integration of all or part of the transformation vector into the genome of the filamentous fungus. However, transformation resulting in the maintenance of a self-replicating extra-chromosomal transformation vector is also contemplated. Many standard transfection methods can be used to produce filamentous fungal cell lines that express large quantities of the heterologous protein, and as such, any of the known procedures for introducing foreign nucleotide sequences into fungal host cells may be used. These include the use of calcium phosphate transfection, polybrene, protoplast fusion, electroporation, biolistics, liposomes, microinjection, plasma vectors, viral vectors and any of the other known methods for introducing cloned genomic DNA, cDNA, synthetic DNA or other foreign genetic material into a host cell. Also of use is the Agrobacterium-mediated transfection method such as the one described in U.S. Pat. No. 6,255,115.

After the expression vector is introduced into the cells, the transformed cells are cultured under conditions favoring expression of gene. Large batches of transformed cells can be cultured as described herein. Finally, the protein product is recovered from the culture using standard techniques. Thus, the disclosure provides for the expression and enhanced production of desired proteins of interest, as described herein.

In certain one or more embodiments or aspects of the disclosure, filamentous fungal cells (strains) may comprise one or more genetic modifications, including, but is not limited to, (a) the introduction, substitution, or removal of one or more nucleotides in a gene (gene CDSs, or ORF thereof), or the introduction, substitution, or removal of one or more nucleotides in a regulatory element required for the transcription or translation of the gene (gene CDS or ORF), (b) a gene disruption, (c) a gene conversion, (d) a gene deletion, (e) a gene down-regulation, (f) specific mutagenesis and/or (g) random mutagenesis of a gene (gene CDS or ORF thereof).

As generally set forth above and described hereinafter, one skilled in the art may readily perform one or more genetic modifications and construct recombinant/modified/variant filamentous fungal strains thereof, by reference to one or more nucleic acid sequences and/or protein sequence disclosed herein. For example, gene deletion techniques enable the partial or complete removal of the gene, thereby eliminating or reducing expression/production of the protein, and/or thereby eliminating or reducing expression/production the encoded protein. In such methods, the deletion of the gene may be accomplished by homologous recombination using an integration plasmid/vector that has been constructed to contiguously contain the 5′ and 3′ regions flanking the gene. The contiguous 5′ and 3′ regions may be introduced into a filamentous fungal cell, for example, on an integrative plasmid/vector in association with a selectable marker to allow the plasmid to become integrated in the cell.

In other embodiments, a modified strain of filamentous fungus comprises genetic modifications which disrupt or inactivate a gene of interest. Exemplary methods of gene disruption/inactivation include disrupting any portion of the gene, including the polypeptide coding sequence (CDS), promoter, enhancer, or another regulatory element, which disruption includes substitutions, insertions, deletions, inversions, and combinations thereof and variations thereof. A non-limiting example of a gene disruption technique includes inserting (integrating) into one or more of the genes of the disclosure an integrative plasmid containing a nucleic acid fragment homologous to the gene of interest, which will create a duplication of the region of homology and incorporate (insert) vector DNA between the duplicated regions. In certain other non-limiting examples, a gene disruption technique includes inserting into a gene of interest an integrative plasmid containing a nucleic acid fragment homologous to the gene of interest, which will create a duplication of the region of homology and incorporate (insert) vector DNA between the duplicated regions, wherein the vector DNA inserted separates, e.g., the promoter of the gene from the protein coding region, or interrupts (disrupts) the coding, or non-coding, sequence of the gene, resulting in an enhanced protein productivity phenotype. A disrupting construct may be a selectable marker gene (e.g., pyr2) accompanied by 5′ and 3′ regions homologous to the gene of interest. The selectable marker enables identification of transformants containing the disrupted gene. Thus, in certain embodiments, gene disruption includes modification of control elements of the gene, such as the promoter, ribosomal binding site (RBS), untranslated regions (UTRs), codon changes, and the like.

In other embodiments, a modified strain of filamentous fungus is constructed (i.e., genetically modified) by introducing, substituting, or removing one or more nucleotides in the gene, or a regulatory element required for the transcription or translation thereof. For example, nucleotides may be inserted or removed so as to result in the introduction of a pre-mature stop codon, the removal of the start codon, or a frame-shift of the open reading frame (ORF). Such a modification may be accomplished by site-directed mutagenesis or PCR generated mutagenesis in accordance with methods known in the art.

In other embodiments, a modified strain of filamentous fungus is constructed by the process of gene conversion. For example, in the gene conversion method, a nucleic acid sequence corresponding to the target gene is mutagenized in vitro to produce a defective nucleic acid sequence, which is then transformed into the parental cell to produce a variant cell comprising a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is affected by selection for the marker under conditions not permitting plasmid replication. Selection for a second recombination event leading to gene replacement is affected by examination of colonies for loss of the selectable marker and acquisition of the mutated gene.

In other embodiments, a modified strain of filamentous fungus is constructed by established anti-sense (gene-silencing) techniques, using a nucleotide sequence complementary to the nucleic acid sequence of the gene of interest. More specifically, expression of a gene by a filamentous fungus strain may be reduced (down-regulated) or eliminated by introducing a nucleotide sequence complementary to the nucleic acid sequence of the gene, which is transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to, RNA interference (RNAi), small interfering RNA (siRNA), microRNA (miRNA), antisense oligonucleotides, and the like, all of which are well known to the skilled artisan.

In other embodiments, a modified strain of filamentous fungus is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions and selecting for mutant cells exhibiting reduced or no expression of the gene.

In certain other embodiments, a modified strain of filamentous fungus is constructed by means of site-specific gene editing techniques. For example, in certain embodiments, a variant strain of filamentous fungus is constructed (i.e., genetically modified) by use of transcriptional activator like endonucleases (TALENs), zinc-finger endonucleases (ZFNs), homing (mega) endonuclease and the like. More particularly, the portion of the gene to be modified (e.g., a coding region, a non-coding region, a leader sequence, a pro-peptide sequence, a signal sequence, a transcription terminator, a transcriptional activator, or other regulatory elements required for expression of the coding region) is subjected genetic modification by means of ZFN gene editing, TALEN gene editing, homing (mega) endonuclease and the like, which modification methods are well known and available to one skilled in the art.

In certain other embodiments, a modified strain of filamentous fungus is constructed by means of CRISPR/Cas9 editing. More specifically, compositions and methods for fungal genome modification by CRISPR/Cas9 systems are described and well known in the art (e.g., see, PCT Publication Nos: WO2016/100571, WO2016/100568, WO2016/100272, WO2016/100562 and the like). Thus, a gene of interest can be disrupted, deleted, mutated or otherwise genetically modified by means of nucleic acid guided endonucleases, that find their target DNA by binding either a guide RNA (e.g., Cas9) or a guide DNA (e.g., NgAgo), which recruits the endonuclease to the target sequence on the DNA, wherein the endonuclease can generate a single or double stranded break in the DNA. This targeted DNA break becomes a substrate for DNA repair and can recombine with a provided editing template to disrupt or delete the gene. For example, the gene encoding the nucleic acid guided endonuclease (e.g., a Cas9 from S. pyogenes, or a codon optimized gene encoding the Cas9 nuclease) is operably linked to a promoter active in the filamentous fungal cell and a terminator active in filamentous fungal cell, thereby creating a filamentous fungal Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a filamentous fungal expression cassette for the gRNA is created by operably linking the DNA encoding the gRNA to a promoter active in filamentous fungal cells and a terminator active in filamentous fungal cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the filamentous fungal host's machinery to repair the DNA break generated by the RGEN (RNA-guided endonuclease).

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR, by amplifying the target locus with a forward and reverse primer. These primers can amplify the wild-type locus or the modified locus that has been edited by the RGEN. These fragments are then sequenced using a sequencing primer to identify edited colonies.

Another way in which a gene of interest can be genetically modified is by altering the expression level of the gene of interest. For example, nuclease-defective variants of such nucleotide-guided endonucleases (e.g., Cas9 D10A, N863A or Cas9 D10A, H840A) can be used to modulate gene expression levels by enhancing or antagonizing transcription of the target gene. These Cas9 variants are inactive for all nuclease domains present in the protein sequence but retain the RNA-guided DNA binding activity (i.e., these Cas9 variants are unable to cleave either strand of DNA when bound to the cognate target site). Thus, the nuclease-defective proteins (i.e., Cas9 variants) can be expressed as a filamentous fungus expression cassette and when combined with a filamentous fungus gRNA expression cassette, such that the Cas9 variant protein is directed to a specific target sequence within the cell. The binding of the Cas9 (variant) protein to specific gene target sites can block the binding or movement of transcription machinery on the DNA of the cell, thereby decreasing the amount of a gene product produced. Thus, any of the genes disclosed herein can be targeted for reduced gene expression using this method. Gene silencing can be monitored in cells containing the nuclease defective Cas9 expression cassette and the gRNA expression cassette(s) by using methods such as RNAseq.

### IV. Fermentation and Recovery of Globin Proteins

As briefly stated in the preceding sections, the present strains and methods find use in the production of commercially important globin proteins in submerged cultures of filamentous fungi. For example, in certain embodiments, a recombinant (modified) filamentous fungal cell comprising an introduced expression cassette produces at least about 30 grams total protein per liter of broth (g/L) when fermented under suitable conditions for the production of the globin protein. In other one or more embodiments, a modified filamentous fungal cell comprising an introduced expression cassette produces at least about 0.5 grams of globin protein per liter of broth (g/L), when fermented under suitable conditions for the production of the globin protein. Thus, in certain embodiments, total protein titer and globin protein titer may be defined as the amount of total protein per volume (g/L) and the amount of globin protein per volume (g/L), respectively. For example, protein titers can be measured by methods known in the art (e.g., ELISA, HPLC, Bradford assay, LC/MS and the like).

In certain other embodiments, a modified filamentous fungal cell comprising an introduced cassette may be described by volumetric productivity, which is defined as the amount of protein produced (g) during the fermentation per nominal volume (L) of the bioreactor per total fermentation time (h). For example, volumetric productivities can be measured by methods known in the art (e.g., ELISA, HPLC, Bradford assay, LC/MS and the like).

In certain other embodiments, a modified filamentous fungal cell comprising an introduced cassette may be described according to total protein yield, wherein total protein yield is defined as the amount of protein produced (g) per gram of carbohydrate fed, relative to the (unmodified) parental strain. Thus, as used herein, total protein yield (g/g) may be calculated using the following equation:

$$Yf = Tp / Tc$$

wherein “Yf” is total protein yield (g/g), “Tp” is the total protein produced during the fermentation (g) and “Tc” is the total carbohydrate (g) fed during the fermentation (bioreactor) run.

Total protein yield may also be described as carbon conversion efficiency/carbon yield, for example, as in the percentage (%) of carbon fed that is incorporated into total protein. Thus, in certain embodiments, a modified filamentous fungal cell comprising an introduced cassette may be described according to carbon conversion efficiency (e.g., an increase in the percentage (%) of carbon fed that is incorporated into total protein).

In certain other embodiments, a modified filamentous fungal cell comprising an introduced cassette may be described according to specific productivity (Qp) of the globin protein. For example, the detection of specific productivity (Qp) is a suitable method for evaluating rate of globin protein production, wherein the Qp can be determined using the following equation:

$$“ Qp = g P / g DCW \cdot hr ”$$

wherein, “gP” is grams of protein produced in the tank; “gDCW” is grams of dry cell weight (DCW) in the tank and “hr” is fermentation time in hours from the time of inoculation, which includes the time of production as well as growth time.

In certain embodiments, the disclosure provides, inter alia, compositions and methods for producing globin proteins comprising fermenting a filamentous fungal cell comprising one or more introduced globin protein cassettes, wherein the fungal cell expresses and secrets (i.e., produces) the globin proteins. In general, fermentation methods well known in the art are used to ferment the fungal cells. In some embodiments, the fungal cells are grown under batch or continuous fermentation conditions.

A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen, and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. With filamentous fungi, the pH normally is within the range of about 2.5 to 8.0; with T. reesei, the pH normally is within the range of about 3.0 to 7.0. Preferences for pH range of microorganisms are dependent on the media employed to some extent, as well as the particular microorganism, and thus change somewhat with change in media as can be readily determined by those skilled in the art. [0170] In certain aspects, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

The fermentation can be conducted as a batch or continuous operation, fed batch operation may be preferred for ease of control, production of uniform quantities of products, and most economical uses of all equipment.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

The collection and purification of globin proteins from the fermentation broth can be done by procedures known to one of skill in the art. For instance, as described above, the recombinant fungal strains of the disclosure can be constructed to secret one or more globin proteins into the fermentation broth, simplifying the globin protein recovery process (e.g., no cell lysis required), thereby reducing costs of globin protein production. The fermentation broth will generally contain cellular debris, including cells, various suspended solids and other biomass contaminants, as well as the desired globin proteins, which are removed from the fermentation broth by means known in the art.

In certain other one or more embodiments or aspects, purified globin (protein) preparations may be derived or recovered from fermentation broths collected and harvested.

As used herein, the terms “purified”, “isolated” or “enriched” with regard to a globin (protein) means that the globin is transformed from a less pure state by virtue of separating it from some, or all of, the contaminants with which it is associated. Contaminants include, but are not limited to, microbial cells, metabolites, solvents, chemicals, color, aggregates, process aids, inhibitors, fermentation media, cell debris, nucleic acids, proteins other than the target leghemoglobin, host cell proteins, cross-contaminants from the production equipment and the like.

Thus, in the context of a “purified globin” as used herein, purification may be accomplished by any art-recognized separation techniques, including, but not limited to, ion exchange chromatography, affinity chromatography, hydrophobic separation, dialysis, protease treatment, heat treatment, ammonium sulphate precipitation or other protein salt precipitation, crystallization, centrifugation, size exclusion chromatography, filtration, microfiltration, gel electrophoresis, or separation on a gradient to remove whole cells, cell debris, impurities, extraneous proteins, or enzymes undesired in the final composition.

It is further possible to then add constituents to a purified or isolated globin composition which provide additional benefits, for example, activating agents, anti-inhibition agents, desirable ions, compounds to control pH or other enzymes or chemicals.

As used herein, globin “purity” is a relative term, and is not meant to be limiting, when used in phrases such as a “recovered globin is of higher purity, the same purity, or lower purity than prior to the recovery process”. For example, the relative “purity” of a globin (protein), before and after a recovery process, may be determined using methods known in the art, including but not limited to, general quantification methods (e.g., Bradford, UV-Vis, activity assays), electrophoretic analysis (SDS-PAGE), analytical HPLC, mass spectrometry, hydrophobic interaction chromatography and the like.

Non-limiting examples for accessing the relative purity of a globin, include, but are not limited to, SDS-PAGE analysis and/or the A₂₈₀ ratio of non-globin (impurities) relative to the globin. For example, the relative globin purity via SDS-PAGE can be determined by visual abundance of globin (protein) band compared to non-globin protein (unwanted contaminants; impurities) bands present in the preparation. Alternatively, the relative purity of a globin can be determined by the non-globin to globin (A₂₈₀) ratio.

For example, the A₂₈₀ ratio is a measure of amount of 280 nm absorbance contributed by non-globin impurities for 1 unit 280 nm absorbance contributed by globin in a protein preparation (e.g., non-globin A₂₈₀/globin A₂₈₀), wherein a smaller number means higher purity. More particularly, the method for determining the globin (A₂₈₀) concentration can be measured by HPLC using a purified globin as the standard, wherein the concentration by HPLC is converted to globin A₂₈₀ using 1 mg/mL globin=1.00 at 280 nm absorbance. The method for determining the non-globin (A₂₈₀) concentration in the preparation can be measured using a 1 cm path glass cuvette zeroed with MilliQ water, diluted to A₂₈₀<1 with MilliQ water as needed, wherein non-globin A₂₈₀ concentration is calculated by subtracting the globin A₂₈₀ from the preparation measurement.

Thus, in one or more embodiments or aspects of the disclosure, globin proteins are recovered from the fermentation broths of filamentous fungal cells fermented under suitable conditions for the production of the globin proteins. In certain embodiments, a filamentous fungal host cell are constructed for the secretion of globin protein into the fermentation broth, wherein the globin proteins are recovered from the end of fermentation (EOF) broth. In certain other embodiments, such as when a filamentous fungal host cell has been constructed for intracellular globin expression, the EOF fermentation broth is subjected to a cell lysis process, wherein the globin proteins are recovered from the lysed cell broth.

Thus, in certain one or more embodiments or aspects, recovered globins are of higher purity after performing one or more recovery processes described herein. For example, a fermentation broth (e.g., a whole broth at the end of fermentation) may be subjected to one or more protein recovery processes including, but not limited to, broth conditioning processes, broth clarification processes, protein enrichment and/or protein purification processes (e.g., protein concentration, filtration, precipitation, crystallization, crystal separation, crystal sludge dissolution processes and the like), buffer exchange processes, sterile filtration processes and the like. In certain aspects, the fermentation broth is subjected to a broth treatment (broth conditioning) process to improve subsequent broth handling properties. In certain other embodiments, such as when a filamentous fungal host cell has been constructed for intracellular globin expression, the fermentation broth is subjected to a cell lysis process prior to recovering the globin. Cell lysis processes include without limitation, enzymatic treatments (e.g., lysozyme, proteinase K treatments), chemical means (e.g., ionic liquids), physical means (e.g., French pressing, ultrasonic), simply holding culture without feeds, and the like.

Thus, as described herein, the methods/processes of the disclosure are not meant to be limiting, as one of skill may readily adapt or modify one or more of the compositions and/or methods disclosed herein for the recovery of specific globin proteins, and/or combinations thereof. In certain aspects, a fermentation broth obtained by fermenting filamentous fungal cells expressing and secreted globin proteins can be processed by harvesting, clarifying and concentrating the broth, as generally described herein.

### V. Exemplary Embodiments

Non-limiting embodiments of the disclosure include, but are not limited to:
1. A recombinant filamentous fungal cell expressing a heterologous globin protein.
2. A recombinant filamentous fungal cell expressing and secreting a heterologous globin protein when fermented under suitable conditions.
3. The recombinant cell of embodiment 1 or embodiment 2, selected from the group consisting of an Acremonium sp. cell, an Aspergillus sp. cell, an Emericella sp. cell, a Fusarium sp. cell, a Humicola sp. cell, a Mucor sp. cell, a Myceliophthora sp. cell, a Neurospora sp. cell, a Penicillium sp. cell, a Scytalidium sp. cell, a Talaromyces sp. cell, a Thielavia sp. cell, a Tolypocladium sp. cell and a Trichoderma sp. cell.
4. The recombinant cell of any one of embodiments 1-3, comprising an introduced expression cassette encoding the globin protein, wherein the cassette comprises an upstream (5′) promoter (pro) sequence operably linked to a downstream nucleic acid (sig-seq) encoding a protein (signal) secretion sequence operably linked to a downstream (3′) nucleic acid (globin CDS) encoding the globin protein.
5. The recombinant cell of embodiment 4, wherein the nucleic acid (globin CDS) encoding the globin protein comprises an upstream (5′) nucleic acid (N-fusion) encoding a N-terminal protein fusion.
6. The recombinant cell of embodiment 4, wherein the nucleic acid (globin CDS) encoding the globin protein comprises an upstream (5′) nucleic acid (N-linker) encoding a N-terminal protein cleavage site.
7. The recombinant cell of embodiment 4, wherein the nucleic acid (globin CDS) encoding the globin protein comprises a downstream (3′) nucleic acid (C-fusion) encoding a C-terminal protein fusion.
8. The recombinant cell of embodiment 4, wherein the nucleic acid (globin CDS) encoding the globin protein comprises a downstream (3′) nucleic acid (C-linker) encoding a C-terminal protein cleavage site.
9. The recombinant cell of embodiment 4, wherein the nucleic acid (globin CDS) encoding the globin protein comprises an upstream (5′) nucleic acid (N-fusion) encoding a N-terminal protein fusion and a downstream (3′) nucleic acid (C-fusion) encoding a C-terminal protein fusion.
10. The recombinant cell of embodiment 4, wherein the nucleic acid (globin CDS) encoding the globin protein comprises an upstream (5′) nucleic acid (N-linker) encoding a N-terminal protein cleavage site and a downstream (3′) nucleic acid (C-linker) encoding a C-terminal protein cleavage site.
11. The recombinant cell of any one of claims 4-10, wherein the nucleic acid (globin CDS) encoding the globin protein comprises a combination of upstream nucleic acids encoding a N-terminal protein fusion and a N-terminal protein cleavage site in either order, and/or a combination of downstream nucleic acids encoding a C-terminal protein fusion and C-terminal protein cleavage site in either order.
12. The recombinant cell of any one of embodiments 4-11, wherein the cassette further comprises a terminator (term) sequence operably linked and positioned at the 3′ end of the cassette.
13. The recombinant cell of any one of embodiments 4-12, wherein the cassette is integrated into the genome of the cell.
14. The recombinant cell of any one of embodiments 4-13, comprising at least two introduced polynucleotides encoding the same or different globin proteins and/or at least two introduced expression cassettes encoding the same or different globin proteins.
15. The recombinant cell of any one of embodiments 1-14, further comprising an introduced expression cassette encoding a protease inhibitor.
16. The recombinant cell of embodiment 15, wherein the protease inhibitor is selected from the group consisting of a native barley amylase subtilisin inhibitor (BASI) protein and functional variants thereof, a native soybean trypsin inhibitor (STI) protein and functional variants thereof, a native Bowman-Birk inhibitor (BBI) protein and functional variants thereof, a native Kunitz-type inhibitor (KTI) protein and functional variants thereof, a native potato metallo-carboxypeptidase inhibitor protein and functional variants thereof.
17. The recombinant cell of any one of embodiments 1-16, further comprising deletions of one or more endogenous genes encoding one or more secreted proteases.
18. The recombinant cell of embodiment 17, wherein the one or more proteases are selected from the group consisting of a subtilisin-like serine protease, an aspartic protease, a trypsin-like serine protease, a glutamic protease, and an aminopeptidase.
19. The recombinant cell any one of embodiments 1-18, fermented for at least about 96 hours to about 300 hours.
20. The recombinant cell of embodiment 19, wherein the cell produces at least 0.1 grams of globin protein per liter of fermentation broth (g/L).
21. The recombinant cell of embodiment 19, wherein the cell produces at least 0.2 to 0.5 grams of globin protein per liter of fermentation broth (g/L).
22. The recombinant cell of embodiment 19, fermented for about 180-190 hours.
23. The recombinant cell of embodiment 22, wherein the cell produces at least 1 grams of globin protein per liter of fermentation broth (g/L).
24. The recombinant cell of embodiment 1 or embodiment 2, wherein the globin protein is selected from the group consisting of leghemoglobins, myoglobins, hemoglobins, cyanoglobins and non-symbiotic hemoglobins.
25. The recombinant cell of embodiment 24, wherein the leghemoglobin protein comprises at least about 60% to 100% sequence identity to the native soybean leghemoglobin protein (SEQ ID NO: 1) or at least 60% to 100% sequence identity to the native French bean leghemoglobin protein (SEQ ID NO: 19).
26. The recombinant cell of embodiment 24, wherein the myoglobin protein comprises at least about 60% to 100% sequence identity to the native bovine myoglobin protein (SEQ ID NO: 3).
27. The recombinant cell of embodiment 24, wherein the globin protein comprises a globin superfamily domain and a functional porphyrin (heme) binding site.
28. A method for producing a heterologous globin protein in a filamentous fungal cell comprising: introducing an expression cassette encoding a globin protein into the filamentous fungal cell, wherein the cassette comprises an upstream (5′) promoter (pro) sequence operably linked to a downstream nucleic acid (sig-seq) encoding a protein (signal) secretion sequence operably linked to a downstream (3′) nucleic acid (globin CDS) encoding the globin protein, and fermenting the modified cell under suitable conditions for the production of the globin protein.
29. The method of embodiment 28, wherein the filamentous fungal cell is selected from the group consisting of an Acremonium sp. cell, an Aspergillus sp. cell, an Emericella sp. cell, a Fusarium sp. cell, a Humicola sp. cell, a Mucor sp. cell, a Myceliophthora sp. cell, a Neurospora sp. cell, a Penicillium sp. cell, a Scytalidium sp. cell, a Talaromyces sp. cell, a Thielavia sp. cell, a Tolypocladium sp. cell and a Trichoderma sp. cell.
30. The method of embodiment 28, wherein the nucleic acid (globin CDS) encoding the globin protein comprises an upstream (5′) nucleic acid (N-fusion) encoding a N-terminal protein fusion.
31. The method of embodiment 28, wherein the nucleic acid (globin CDS) encoding the globin protein comprises an upstream (5′) nucleic acid (N-linker) encoding a N-terminal protein cleavage site.
32. The method of embodiment 28, wherein the nucleic acid (globin CDS) encoding the globin protein comprises a downstream (3′) nucleic acid (C-fusion) encoding a C-terminal protein fusion.
33. The method of embodiment 28, wherein the nucleic acid (globin CDS) encoding the globin protein comprises a downstream (3′) nucleic acid (C-linker) encoding a C-terminal protein cleavage site.
34. The method of embodiment 28, wherein the nucleic acid (globin CDS) encoding the globin protein comprises an upstream (5′) nucleic acid (N-fusion) encoding a N-terminal protein fusion and a downstream (3′) nucleic acid (C-fusion) encoding a C-terminal protein fusion.
35. The method of embodiment 28, wherein the nucleic acid (globin CDS) encoding the globin protein comprises an upstream (5′) nucleic acid (N-linker) encoding a N-terminal protein cleavage site and a downstream (3′) nucleic acid (C-linker) encoding a C-terminal protein cleavage site.
36. The method of any one of embodiments 28-35, wherein the nucleic acid (globin CDS) encoding the globin protein comprises a combination of upstream nucleic acids encoding a N-terminal protein fusion and a N-terminal protein cleavage site in either order, and/or a combination of downstream nucleic acids encoding a C-terminal protein fusion and C-terminal protein cleavage site in either order.
37. The method of any one of embodiments 28-36, wherein the cassette further comprises a terminator (term) sequence operably linked and positioned at the 3′ end of the cassette.
38. The method of any one of embodiments 28-37, wherein the cassette is integrated into the genome of the cell.
39. The method of any one of embodiments 28-38, comprising at least two introduced polynucleotides encoding the same or different globin proteins and/or comprising at least two introduced expression cassettes encoding the same or different globin proteins.
40. The method of any one of embodiments 28-39, further comprising an introduced expression cassette encoding a protease inhibitor.
41. The method of embodiment 40, wherein the protease inhibitor is selected from the group consisting of a native barley amylase subtilisin inhibitor (BASI) protein and functional variants thereof, a native soybean trypsin inhibitor (STI) protein and functional variants thereof, a native Bowman-Birk inhibitor (BBI) protein and functional variants thereof, a native Kunitz-type inhibitor (KTI) protein and functional variants thereof, a native potato metallo-carboxypeptidase inhibitor protein and functional variants thereof.
42. The method of any one of embodiments 28-41, further comprising deletions of one or more endogenous genes encoding one or more secreted proteases.
43. The method of embodiment 42, wherein the one or more proteases are selected from the group consisting of a subtilisin-like serine protease, an aspartic protease, a trypsin-like serine protease, a glutamic protease, and an aminopeptidase.
44. The method of any one of embodiments 28-43, wherein the cell is fermented for at least about 96 hours to about 300 hours.
45. The method of embodiment 44, wherein the cell produces at least 0.1 grams of globin protein per liter of fermentation broth (g/L).
46. The method of embodiment 44, wherein the cell produces at least 0.2 to 0.5 grams of globin protein per liter of fermentation broth (g/L).
47. The method of embodiment 44, fermented for about 180-190 hours.
48. The method of embodiment 47, wherein the cell produces at least 1 grams of globin protein per liter of fermentation broth (g/L).
49. The method of embodiment 28, wherein fermenting the modified cell under suitable conditions for the production of the globin protein does not require or include hemin media supplementation and/or does not require 5-aminolevulonic acid (ALA) media supplementation.
50. The method of embodiment 28, wherein the modified cell does not require or include heme biosynthesis pathway engineering for the production of the globin protein.
51. The method of any one of embodiments 28-50, wherein the expressed globin is secreted and recovered from the fermentation broth.
52. The method of embodiment 51, wherein the recovered globin protein is purified.
53. The method of embodiment 28, wherein the globin protein is selected from the group consisting of leghemoglobins, myoglobins, hemoglobins, cyanoglobins and non-symbiotic hemoglobins.
54. The method of embodiment 53, wherein the leghemoglobin protein comprises at least about 60% to 100% sequence identity to the native soybean leghemoglobin protein (SEQ ID NO: 1) or comprises at least about 60% to 100% to the native French bean leghemoglobin protein (SEQ ID NO: 19).
55. The method of embodiment 53, wherein the myoglobin protein comprises at least about 60% to 100% sequence identity to the native bovine myoglobin protein (SEQ ID NO: 3).

### EXAMPLES

Certain aspects of the present invention may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Expression and Secretion of Heterologous Leghemoglobin Proteins

As briefly described above, Applicant of the instant disclosure has contemplated, designed, and constructed recombinant (modified) filamentous fungal cells capable of producing heterologous globin proteins. In certain one or more embodiments, recombinant polynucleotides (e.g., expression cassettes) encoding one or more heterologous globin proteins are introduced into a filamentous fungal cell of the disclosure. For example, in certain embodiments, an expression cassette encoding a secreted globin protein comprises (in the 5′ to 3′ direction) a promoter (pro) region sequence operably linked to a nucleic acid (sig-seq) encoding a protein (signal) secretion sequence operably linked to a nucleic acid (globin CDS) encoding the globin protein, and the like.

### A. Construction of Vectors for the Expression of Soybean Leghemoglobin as a Secreted Protein

One strategy for improving the expression of heterologous globin proteins in filamentous fungal strains is to express the gene of interest (GOI) with a signal sequence, and a strong promoter, such as the T. reesei cellobiohydrolase I gene promoter (Pcbh1). In particular, for expression of soybean leghemoglobin C2 gene (NP_001235248.2, GI: 1229203762), the gene was codon optimized for expression in T. reesei (SEQ ID NO: 2). For example, a synthetic DNA sequence comprising the codon optimized leghemoglobin C2 gene (named “LegGm1b”) was synthesized (Twist Biosciences, San Francisco, CA) that comprises a native Cbh1 signal sequence (SEQ ID NO: 13) or a native Pep1 signal sequence (SEQ ID NO: 14) using the GeneArt Seamless Cloning and Assembly Enzyme Mix (Thermo Fisher Scientific, Carlsbad, CA).

The backbone of the expression vector was amplified from a plasmid that comprises the following features: a 1 kb upstream (5′) flanking homology sequence suitable for integration into a genomic locus of the T. reesei strain, a cbh1 promoter (Pcbh1) sequence (SEQ ID NO: 11), a Cbh1 protein secretion sequence (SEQ ID NO:13) or a Pep1 protein secretion sequence (SEQ ID NO:14), a cbh1 terminator (Tcbh1) region sequence (SEQ ID NO:15), a T. reesei pyr2 gene marker sequence (SEQ ID NO:16) for transformation in T. reesei, a 1 kb downstream (3′) flanking homology suitable for integration into a genomic locus of the T. reesei strain, and bacterial vector sequences for the selection and maintenance of the plasmid in E. coli. The synthetic DNA encoding the leghemoglobin (LegGm1b) contains a 25 bp 5′ flanking sequence that overlaps with cbh1 promoter (Pcbh1) region and contains a 25 bp 3′ flanking sequence that overlaps with the cbh1 terminator (Tcbh1) region. This vector construction generated leghemoglobin expression vectors named pCHL853 (pI1-Pcbh1-LegGm1b; SEQ ID NO: 36) and pCHL856 (pI1-Pcbh1-LegGm1b-pep1ss; SEQ ID NO: 37), as shown below in TABLE 1.

### B. Construction of Vectors for the Expression of French Bean Leghemoglobin as a Secreted Protein

The French bean leghemoglobin (SEQ ID: 19, NCBI Accession: AAA33767.1) is a second exemplary globin protein, wherein Applicant has constructed and screened recombinant filamentous fungal strains for the expression and secretion the French bean leghemoglobin protein. More particularly, a synthetic DNA sequence encoding the French bean leghemoglobin (LegPv1b; SEQ ID NO: 19), wherein SEQ ID NO: 19 has been codon optimized for expression in T. reesei cells. For example, the French bean leghemoglobin (LegPv1b) was synthesized (Twist Biosciences), and the expression construct was built similarly as those for LegGm1b into pCHL852.

### C. Construction of Vectors for the Expression of Soybean Leghemoglobin as a Secreted Fusion Protein

Another strategy for improving expression/production of heterologous globin proteins in filamentous fungal strains is to express the globin gene of interest (GOI) with an N-terminus fusion protein, typically a highly expressed native filamentous fungal protein, such as T. reesei lignocellulosic degrading enzymes Cbh1, Cbh2, Eg1, Eg2, Glucoamylases, and the like. The globin GOI encoding the globin protein can be linked (fused) to a gene of a highly expressed native filamentous fungal protein (e.g., Cbh1) via linker peptide sequences that can be cleaved by native proteases (e.g., Kex2 site; SEQ ID NO: 18; Goller et al. 1998) before secretion. The globin protein can also be linked to other stable domains of highly expressed filamentous fungal proteins, such as the Cbh1 core domain (i.e., Cbh1 without the C-terminus cellulose binding domain; SEQ ID NO: 7).

For the expression of soybean leghemoglobin and bovine myoglobin, either the Cbh1 full-length, mature protein (Cbh1 FL; SEQ ID NO: 9) or the Cbh1 core protein domain (Cbh1 core; SEQ ID NO: 7) can be used as exemplary N-terminus fusion partners (see TABLE 1). The leghemoglobin C2 gene was codon optimized for the expression in T. reesei. A synthetic DNA sequence comprising the codon optimized leghemoglobin C2 gene (LegGm1b; SEQ ID NO: 2) was synthesized (Twist Biosciences, San Francisco, CA), which comprises upstream (5′) and downstream (3′) flanking sequences for construction of leghemoglobin fusion protein expression vectors, using the GeneArt Seamless Cloning and Assembly Enzyme Mix (Thermo Fisher Scientific, Carlsbad, CA). More particularly, the backbone of the expression vector was amplified from a plasmid that comprises the following features: a 1 kb upstream (5′) flanking homology sequence suitable for integration into a pre-selected genomic locus “TrC114F”, located at the targeting site of the single guide RNA sgRNA-TrC144F (SEQ ID NO: 45; 5′-GCUUUCGCCUUACUUCUGCAGGG-3′) (Synthego, Redwood City, CA) of the T. reesei strain, a cbh1 promoter (Pcbh1) region sequence (SEQ ID NO: 11) a DNA sequence encoding a Cbh1 secretion signal (SEQ ID NO: 13), a DNA sequence encoding a Cbh1 protein core domain (Cbh1 core; (SEQ ID NO: 7), DNA sequence encoding a Kex2 protease cleavage site (SEQ ID NO: 18) and a cbh1 terminator (Tcbh1) region sequence (SEQ ID NO: 15). A T. reesei pyr2 gene (marker) cassette as a selection marker for transformation in T. reesei, the 1 kb 3′ flanking homology sequence downstream of the “TrC144F” locus, and bacterial vector sequences for the selection and maintenance of the plasmid in E. coli. The synthetic DNA sequence (LegGm1b; SEQ ID NO: 2) encoding the leghemoglobin protein (SEQ ID NO: 1) contains a 25-base pair (bp) upstream (5′) flanking sequence that overlaps with the Cbh1 core-KEX2 region, and a 25-bp downstream (3′) flanking sequence that overlaps with the cbh1 terminator (Tcbh1) region. This vector construction generated the leghemoglobin expression vector named “pLH1088” (SEQ ID NO: 38).

A second leghemoglobin expression vector was constructed for the expression of a fusion leghemoglobin protein with the complete (full-length, mature) Cbh1 protein. This vector comprises the following features: a 1 kilobase (kb) upstream (5′) flanking homology sequence at the “TrC114F” locus as described above, the cbh1 promoter region sequence (SEQ ID NO: 11), the full-length Cbh1 sequence (Cbh1 FL) that includes the Cbh1 signal sequence, as well as the C-terminus cellulose binding domain, the KEX2 sequence (SEQ ID NO: 18), followed by the leghemoglobin gene CDS (LegGm1b), the cbh1 terminator region (SEQ ID NO: 15), and a 1 kb downstream (3′) flanking homology sequence at the genomic locus “TrC114F”. The rest of the expression vector contains T. reesei pyr2 gene and bacterial vector sequences for the selection and maintenance of the plasmid in E. coli. The resulting expression vector was named “pLH1104” (SEQ ID NO: 39).

A similar vector was constructed with the same sequence as pLH1104, except that the leghemoglobin was expressed with both an N-terminus fusion of Cbh1 and a C-terminus fusion of a 6-His tag which vector was named “pLH1105” (SEQ ID NO: 40).

### Example 2

### Expression and Secretion of Heterologous Bovine Myoglobin Proteins

The bovine myoglobin gene (Mb; NP_776306.1, GI: 27806939) was codon optimized (Mb1b, SEQ ID NO: 4) for the expression in T. reesei, and the synthetic DNA sequence fragment containing Mb1b was obtained from Twist Biosciences. A Mb1b expression vector was constructed for the expression of a fusion protein with the full-length Cbh1 (Cbh1 FL) protein. This vector comprises the following features: a 1 kb 5′ flanking homology sequence at the targeted genomic locus, the Pcbh1 promoter, the full-length Cbh1 sequence (Cbh1 FL), the KEX2 sequence, followed by the synthetic Mb1b coding sequence, the Tcbh1 terminator, the pyr2 selection marker, and a 1 kb 3′ flanking homology sequence at the targeted genomic locus. The rest of the expression vector contains bacterial vector sequences for the selection and maintenance of the plasmid in E. coli. The full-length Cbh1-myoglobin (fusion) protein expression vector (Cbh1FL-Mb1b) was named “pLH1106” (pI1-Pcbh1-Cbh1FL-Mb1b) and the full-length Cbh1-myoglobin-His6 (fusion) protein expression vector was named “pLH1107” (pI1-Pcbh1-Cbh1FL-Mb1b-His6).

TABLE 1
GLOBIN EXPRESSION CASSETTE GENETIC ELEMENTS

| Name (Abbreviation) | Description/Function | SEQ ID |
|---|---|---|
| cbh1 promoter (Pcbh1) | Promote transcription of cbh1, LegGm1b, or Mb1b | 11 |
| cbh2 promoter (Pcbh2) | Promote transcription of BASI | 12 |
| Cbh1 signal sequence (cbh1ss) | Secretion of Cbh1, leghemoglobin or myoglobin | 13 |
| Pep1 signal sequence (pep1ss) | Modified Pep1 signal sequence (with an intron sequence | 14 |
| w/gla1 intron | from T. reesei glucoamylase gene for secretion of Cbh1, | |
| | leghemoglobin or myoglobin | |
| cbh1 core protein | Gene CDS of Cbh1 core protein | 8 |
| cbh1 Full-Length | Gene CDS full-length Cbh1 protein | 10 |
| Codon optimized LegGm1b | Codon optimized for expression in T. reesei cells | 2 |
| Codon optimized Mb1b | Codon optimized for expression in T. reesei cells | 4 |
| cbh1 terminator (Tcbh1) | Promote termination of transcription | 15 |
| Dyr2 | Selection marker for isolating transformants | 16 |
| amdS | Selection marker for isolating transformants | 17 |
| BASI | BASI Gene CDS, Inhibits proteolysis of secreted proteins | 6 |
| KEX2 | Modified protease cleavage sequence that improves | 18 |
| | cleavage of fusion protein by Kex2 protease. It contains a | |
| | pre-sequence of VAVE that precedes the dipeptide KR | |
| | sequences, which is the canonical recognition site for Kex2 | |
| | protease. | |

TABLE 2
CHROMOSOME INTEGRATION VECTORS FOR EXPRESSION OF LEGHEMOGLOBIN,
MYOGLOBIN AND BASI

| Plasmid | Plasmid Description | | | N-term | Gene | C-term | Integration | PCR 5′ | PCR 3′ |
|---|---|---|---|---|---|---|---|---|---|
| Name | (5′ → 3′) | Pro | Signal | Fusion | CDS | Fusion | Cassette | Primer | Primer |
| pCHL852 | pI1-Pcbh1-LegPv1b | Pcbh1 | Cbh1 | None | LegPv1b | None | HG10 | OT4268 | OT4269 |
| pCHL853 | pI1-Pcbh1-LegGm1b | Pcbh1 | Cbh1 | None | LegGm1b | None | HG1 | OT4268 | OT4269 |
| pCHL856 | pI1-Pcbh1-LegGm1b- | Pcbh1 | Pep1 | None | LegGm1b | None | HG2 | OT4268 | OT4269 |
| | Pep1ss | | | | | | | | |
| pLH1088 | pI1-Pcbh1-Cbh1core- | Pcbh1 | Cbh1 | Cbh1 core | LegGm1b | None | HG3 | OT4268 | OT4269 |
| | KEX2-LegGm1b | | | | | | | | |
| pLH1104 | pI1-Pcbh1-Cbh1- | Pcbh1 | Cbh1 | Cbh1 FL | LegGm1b | None | HG4 | OT4268 | OT4269 |
| | KEX2-LegGm1b | | | | | | | | |
| pLH1105 | pI1-Pcbh1-Cbh1- | Pcbh1 | Cbh1 | Cbh1 FL | LegGm1b | 6-His | HG5 | OT4268 | OT4269 |
| | KEX2-LegGm1b-His6 | | | | | | | | |
| pLH1106 | pI1-Pcbh1-Cbh1- | Pcbh1 | Cbh1 | Cbh1 FL | Mb1b | None | HG6 | OT4268 | OT4269 |
| | KEX2-Mb1b | | | | | | | | |
| pLH1107 | pI1-Pcbh1-Cbh1- | Pcbh1 | Cbh1 | Cbh1 FL | Mb1b | 6-His | HG7 | OT4268 | OT4269 |
| | KEX2-Mb1b-His6 | | | | | | | | |
| pLH1108 | pKS923-Pcbh2-BASI | Pcbh2 | Pep1 | None | BASI | None | HG8 | OT4337 | OT4338 |
| pLH1109 | pI1c-Pcbh1-BASI- | Pcbh1 | Pep1 | BASI | LegGm1b | None | HG9 | OT4337 | OT4338 |
| | KEX2-LegGm1b | | | | | | | | |

### Example 3

### Cas9 Guided Targeted Integration of Leghemoglobin Expression Cassettes into T. reesei Genome

In the instant example, leghemoglobin and myoglobin expressing T. reesei strains were generated by Cas9 guided targeted integration into the genome, via homologous recombination (HR) or non-homologous end joining (NHEJ) mechanisms. The Cas9-Ribonucleoprotein complex (Cas9RNP) composes of the Cas9 protein and a single chain guide RNA (sgRNA). Upon protoplast transformation of the Cas9RNP complex and the targeted integration cassette into T. reesei cells, the Cas9RNP enters the nucleus via the Nucleus Location Signal (NLS) at the C-terminus of the Cas9 protein. The guide RNA then directs the Cas9RNP to the targeted genomic locus to perform a double stranded cut, which is subsequently repaired by either the integration cassette that contains homologous sequences to both ends of the cutting site, or by various DNA repair mechanisms such as NHEJ (Non-homologous End Joining). For targeting via the homologous recombination approach, typically 50 bp to 1000 bp of sequences that are homologous to both the 5′ and 3′ ends of the integration locus are included in the integration cassette. For the integration cassettes constructed for leghemoglobin or myoglobin, 1 kb of 5′ and 3′ homologous sequences are included to improve the efficiency of chromosomal integration and homology-based recombination at the desired locus.

For the construction of the leghemoglobin and myoglobin expression strains, linear expression cassettes were amplified by PCR using primers OT4268 and OT4269 (see, TABLE 3 below), to generate the DNA fragments that contain 5′ and 3′ one (1) kb flanking sequences for Cas9 targeted chromosomal integration at the chromosome locus.

TABLE 3
PCR PRIMER FOR AMPLIFICATION OF CHROMOSOMAL INTEGRATION CASSETTES

| Primer | Sequence (5′→3′) | Purpose |
|---|---|---|
| OT4268 | TGTGCGAGATCCATGACTGCGTT | 5′ primer for pI1c integration cassette |
| | | at selected target locus (TrC114F) |
| OT4269 | ATAGTATCGTCTTGAGTTATTCCGAGTTCCG | 3′ primer for pI1c integration cassette |
| | | at target locus (TrC114F) |
| OT4337 | CCAACACAAACCTGATCGCGCCAGCTCCGTGCGAAAG | 5′ primer for Pcbh2-BASI cassette |
| OT4338 | TGACGAAGACCTCTAAACAAGTGTACCTGTGCATTC | 3′ primer for Pcbh2-BASI cassette |
| OT4333 | CCCAGATTCCGGCCAGATAG | 5′ diagnostic PCR primer for |
| | | integration cassettes at TrC114F |
| | | locus |
| OT4334 | TCGAACTGTTGTGAGCCCAG | 3′ diagnostic primer PCR for |
| | | integration cassettes at TrC114F |
| | | locus |

The PCR reaction was set up in 6×PCR strips, each strip containing 8 PCR tubes at 50 μL reaction per tube, in a total volume of 1.2 mL. The 5′ PCR primer (OT4268) and the 3′ PCR primer (OT4269) were each added to the final concentration of 0.5 μM, with 0.5 ug/mL of the template DNA plasmids (TABLE 2). The PCR reaction was performed using the NEB-NEXT PCR Master Mix (New England Biolabs, MA), with the following condition: 98° C., 30 seconds; 35 cycles (98° C., 10 seconds; 70° C., 30 seconds; 72° C., 4 minutes); 72° C., 4 minutes. The final PCR products (HG1-HG7, TABLE 2) were digested with DpnI enzyme (New England Biolabs) to remove the plasmid template DNA. The reaction mixture was purified using the Zymo DNA Clean and Concentrator following manufacture's protocols (Zymo Research, Irvine, CA), and dissolved in Elution buffer provided in the kit to 0.5-1.0 μg/L final concentration.

The Cas9RNP complex used for the targeted chromosomal integration of the above expression cassettes was produced as follows: The sgRNA targeting the locus (TrC114F, not including the protospacer adjacent motif (PAM) sequence “GGG”) in the T. reesei genome was obtained from Synthego (South San Francisco, CA), with the RNA sequence of SEQ ID NO: 45 (5′-GCUUUCGCCUUACUUCUGCA-3′), and dissolved to 100 μM in TE buffer (10 mM Tris, 1 mM EDTA, pH 8.0). The Cas9RNP assembly reaction contains 12 μM of Cas9 protein (New England Biolabs), 12 μM of sgRNA-EclipseA, in 1×NEB 3.1 buffer (New England Biolabs). The reaction is incubated at room temperature for 10 minutes and stored on ice until the protoplast transformation is performed. Protoplast transformation of T. reesei host was performed by combining 5 μL of the Cas9RNP complex, 4 μg of the PCR product of the linear integration cassette, and 300 μL of T. reesei protoplasts (108 per mL), following standard procedures. The transformation reactions were plated on Vogel agar media (for selection of the pyr2 marker) and incubated at 32° C. for 5 days. Single colonies were picked from the Vogel agar plates and transferred onto fresh Vogel agar plates and incubated at 32° C. for 3 days.

Colonies were then screened for the expression of leghemoglobin or myoglobin by inoculating 1 mL NREL media (pH 6.5) and growing in Microtiter plates for 4 days at 28° C., with the addition of 1× Halt protease inhibitor cocktail (Thermo Fisher Scientific). SDS-PAGE analysis was performed to screen for transformants that produced the full-length Cbh1 protein (54 kDa), the Cbh1 core domain protein (50 kDa), the leghemoglobin protein (16 kDa), the myoglobin protein (17 kDa), or the 6-histidine tagged fusion proteins. The integration of the linear expression cassettes at the desired locus was screened and verified by colony PCR amplification from the T. reesei transformants using OT4333 and OT4334 as primers.

To assess the reduction (mitigation) of proteolytic degradation of the secreted leghemoglobin and myoglobin proteins by native proteases secreted by T. reesei, an expression cassette for the barley amylase subtilisin (protease) inhibitor (BASI, SEQ ID NO: 6) was integrated into certain strains for the co-expression of leghemoglobin or myoglobin with BASI. The BASI expression cassette comprises a cbh2 promoter region (Pcbh2; SEQ ID NO: 12), a DNA sequence encoding the Pep1 signal sequence (SEQ ID NO: 14), the BASI gene CDS (SEQ ID NO: 6), and a TrpC terminator (TTrpC) region (SEQ ID NO: 46). Using pLH1108 as template, the Pcbh2-BASI cassette was PCR amplified using NEB-NEXT Master Mix (98° C., 30 s; 35 cycles (98° C., 10 s; 70° C., 30 s; 72° C., 4′); 72° C., 4′), using primers OT4337 and OT4338 to yield the 4 kb product (HG8).

To construct a T. reesei strain that co-expresses leghemoglobin and BASI, the linear integration cassettes HG3 from the PCR amplification of pLH1088 (PCR primers OT4268 and OT4269) and HG8 from pLH1108 amplification (PCR primers OT4337 and OT4338) were co-transformed into T. reesei protoplasts together with Cas9RNP-sgRNA-TrC114F. While HG3 cassette is integrated at the locus as confirmed by colony PCR using OT4333 and OT4334 as primers, the HG8 cassette, which does not contain 5′ or 3′ homologous sequences to the T. reesei genome, is integrated randomly into the genome. The transformants were used to inoculate 1 mL. wells in 24-Well Microtiter plates with NREL media (pH 6.5, 1× Halt protease inhibitor cocktail) for 4 days at 28° C., and the expression of Leghemoglobin (or myoglobin) and BASI were analyzed by SDS-PAGE.

### Example 4

### Expression and Production of a Cbh1-Leghemoglobin (N-Terminus) Fusion Protein by Secretion

A fed-batch fermentation run was performed in a two (2) L bioreactor for T. reesei strain BFZ28 (Pcbh1-CBH1 core-KEX2-LegGm1b), as generally described in PCT Publication No. WO2004/035070 (incorporated herein by reference). In particular, cells were first grown in minimal medium containing 75 g/L glucose until glucose is depleted. The production phase was initiated with the combined feeding of glucose and sophorose at pH 6.5 for the induction of protein expression under the control of the cbh1 promoter (Pcbh1), with the addition and 30 g/L VEG-Pro as nutritional supplement. For example, a 10 mL whole broth sample was taken every twenty-four (24) hours and frozen at −20° C., wherein the total fermentation time was about 180 hours. The whole broth samples harvested were thawed and centrifuged. As shown in FIG. 3, the red (dark) color formation in the supernatant indicates secretion of the globin proteins into the culture supernatants. Likewise, the supernatants were analyzed by SDS-PAGE as shown in FIG. 5, wherein the upper protein band of approximately (~) 55 kDa is the Cbh1 core protein and the leghemoglobin protein (~16 KDa) is either co-migrating with the BASI protease inhibitor at ~20 kDa, or present as a minor band at ~17 kDa.

To verify the expression of leghemoglobin, fermentation supernatant was further analyzed by HPLC analysis of the 188-hour supernatant sample (FIG. 6) from the BFZ28 strain fermentation run. The heme prosthetic group of the leghemoglobin protein is detected at 410 nm, which co-migrated with a protein peak detected at 280 nm with a 6.4-minute retention time. The FIG. 6 insert drawing shows the spectral scan of the leghemoglobin peak at 6.4 minutes retention time. This confirms the peak absorbance of the leghemoglobin protein at 410 nm.

### Example 5

### Co-Expression and Production of a Secreted Leghemoglobin and Secreted Protease Inhibitor

Three fed-batch fermentation runs were performed in two (2) L bioreactors for the following three T. reesei strains: (1) BGJ74 (Pcbh1-LegGm1b, Pcbh2-BASI) for direct expression of the soybean leghemoglobin under the cbh1 promoter, (2) BGJ75 (Pcbh1-Pv1b, Pcbh2-BASI) for direct expression of the French bean leghemoglobin under the cbh1 promoter and (3) BGJ76 (Pcbh1-LegGm1b.Pep1ss, Pcbh2-BASI), for direct expression of the soybean leghemoglobin under the cbh1 promoter.

Cells were first grown in minimal medium containing 75 g/L glucose until glucose was depleted. The production phase was initiated with the combined feeding of glucose and sophorose at pH 7 with 30 g/L VEG-Pro for the induction of protein expression under the control of the cbh1 promoter (Pcbh1). A 10 mL whole broth sample was taken every twenty-four (24) hours and frozen at −20° C., wherein the total fermentation time was 188 hours. For example, as presented in FIG. 7, the total soluble proteins secreted in the fermentation run is plotted versus the effective fermentation time (EFT, hours) for the three fermentation runs (strains BGJ74, BGJ75 and BGJ76) described above, together with the data from the previously run BFZ28 fermentation (BFZ28; Pcbh1-CBH1 core-KEX2-LegGm1b) described in Example 4. The fermentation supernatants were analyzed by SDS-PAGE as shown in FIG. 8, wherein the major protein band is detected at 20 kDa, which contains the BASI protease inhibitor and the leghemoglobin protein (~16 kDa) that co-migrates with the BASI protein. The presence of the heme-containing leghemoglobin was further confirmed by HPLC.

To estimate the expression of leghemoglobin in these fermentation runs, the 188-hour supernatant samples from the end of the fermentation runs were analyzed by HPLC (FIG. 9). The heme prosthetic group of the leghemoglobin protein is detected at 410 nm, which co-migrated with a protein peak detected at 280 nm with a 6.4-minute retention time (FIG. 9). The total protein secretion titers at the end of the 188-hour fermentation run are presented below in TABLE 4, with the leghemoglobin levels (g/L) calculated based on the protein peak areas at 280 nm.

TABLE 4
TOTAL SECRETED PROTEIN TITERS

| | Total Secreted | Leghemoglobin |
|---|---|---|
| | Protein Titer | Titer |
| Strain | (g/L) | (g/L) |
| BFZ28 | 50.7 | 2.0 |
| BGJ74 | 36.4 | 2.2 |
| BGJ75 | 41.8 | 1.3 |
| BGJ76 | 54.9 | 3.0 |

### Example 6

### Secreted Myoglobin Production in a Dual-Copy Expression Strain

### A. Construction of a Dual Copy Expression Vector for the Secretion of Bovine Myoglobin

To improve the expression of myoglobin, strains with two differently codon-optimized sequences of the bovine myoglobin gene were constructed. In particular, a tandem-copy expression vector (pLHX143) was constructed and contains two expression cassettes for bovine myoglobin. The first cassette contains the Pcbh1 promoter (SEQ ID NO: 11), a Pep1 signal sequence (Pep1ss, SEQ ID NO: 14), the first codon optimized bovine myoglobin gene (Mb1b, SEQ ID NO: 4), and the terminator sequence of the endoglucanase 1 gene (Tegl1, SEQ ID NO: 50). This sequence is immediately followed by a second cassette that contains the Pcbh2 promoter (SEQ ID NO: 12), a Cbh1 signal sequence (Cbh1ss, SEQ ID NO: 13), a second codon optimized bovine myoglobin gene (Mb1c, SEQ ID NO: 49) encoding for the same bovine myoglobin protein (SEQ ID NO: 3), and the terminator sequence of Cbh1 gene (Tcbh1, SEQ ID NO: 15). This vector also comprises the following features: a 1 kb 5′ flanking homology sequence at the targeted genomic locus, and a 1 kb 3′ flanking homology sequence at the targeted genomic locus. The rest of the expression vector contains bacterial vector sequences for the selection and maintenance of the plasmid in E. coli. This dual copy myoglobin expression vector was named “pLHX143” (pI1-Pcbh1-Mb1b-Pcbh2-Mb1c, SEQ ID NO: 51).

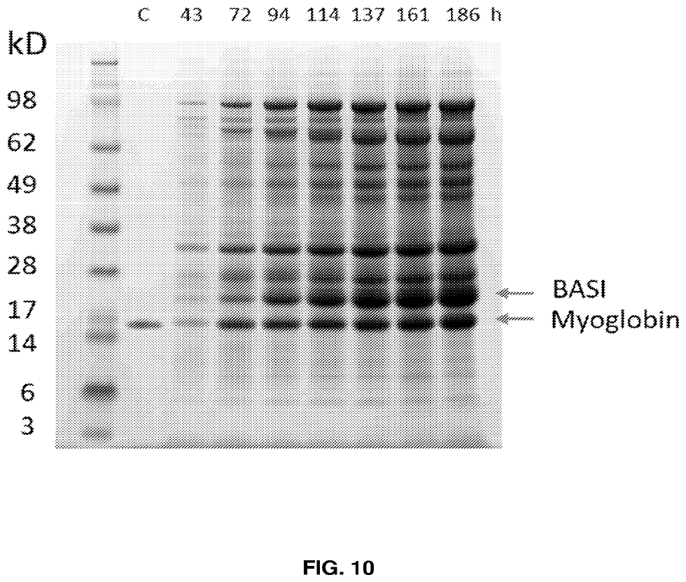

The linear dual copy myoglobin expression cassette was amplified by PCR using primers OT4268 and OT4269 (e.g., see TABLE 3), to generate the DNA fragments that contain 5′ and 3′ one (1) kb flanking sequences for Cas9 targeted chromosomal integration at the chromosome locus. This myoglobin cassette PCR fragment as well as the Pcbh2-BASI cassette PCR fragment were integrated into T. reesei genome as described in Example 3. Colonies were screened for the expression of myoglobin by inoculating 1 mL NREL media (pH 6.5) and growing in microtiter plates for four (4) days at 28° C., without addition of protease inhibitors. SDS-PAGE was performed to analyze the secreted proteins. One of the top expression strains (BHX46) was selected for a one-liter fermentation run, and samples were withdrawn at hours 43, 72, 94, 114, 137, 161, and 186. Culture supernatants from these samples were analyzed by SDS-PAGE (FIG. 10). As shown in FIG. 10, the myoglobin protein was detected as a band at 17 kDa, and the BASI protease inhibitor was detected at 20 kDa. The integration of the linear dual copy myoglobin expression cassette at the desired locus was verified by colony PCR amplification as well as genomic sequencing.

### REFERENCES

PCT Publication No. WO1992/03529
PCT Publication No. WO2004/035070
PCT Publication No. WO2011/153449
PCT Publication No. WO2016/100272
PCT Publication No. WO2016/100562
PCT Publication No. WO2016/100568
PCT Publication No. WO2016/100571
PCT Publication No. WO2021/092356
PCT Publication No. WO2023/278968
U.S. Pat. No. 6,255,115
U.S. Pat. No. 6,268,328
U.S. Pat. No. 7,713,725
U.S. Pat. No. 8,936,917
US Patent Publication No US2014/0024067
US Patent Publication No. US2014/0024067
US Patent Publication No. US2014/0161958
US Patent Publication No. US2021/0289813
U.S. Pat. No. 6,022,725
Cao et al., Science, 9: 991-1001, 2000.
Goller et al., “Role of endoproteolytic dibasic proprotein processing in maturation of secretory proteins in Trichoderma reesei”, Appl Environ Microbiol., 64(9):3202-3208, 1998.
Krainer et al., “Optimizing cofactor availability for the production of recombinant heme peroxidase in Pichia pastoris”, Microbial Cell Factories, 14:4, 2015.
Laskowski and Kato, “Protein Inhibitors of Proteinases”, Ann. Rev. Bioch., 49:593-626, 1980.
Le Marquer et al., “Identification of new signaling peptides through a genome-wide survey of 250 fungal secretomes”, BMC Genomics 20:64 2019.
Shao et al., “High-level secretory production of leghemoglobin in Pichia pastoris through enhanced globin expression and heme biosynthesis”, Bioresource Technology, Volume 363, 2022.
Strickler et al., “Two Novel Streptomyces Protein Protease Inhibitors. Purification, Activity, Cloning and Expression”, The Journal of Biological Chemistry, Vol. 267, No. 5, pages 3236-3241, 1992.
Subramanian et al., “A versatile 2A peptide-based bicistronic protein expressing platform for the industrial cellulase producing fungus, Trichoderma reesei”, Biotechnol for Biofuels 10:34 (2017).

1. A recombinant filamentous fungal cell expressing a protease inhibitor and a heterologous globin protein, wherein the cell secretes the globin protein into the fermentation broth when fermented under suitable conditions.

2. (canceled)

3. The recombinant cell of claim 1, comprising an introduced expression cassette encoding the globin protein, wherein the cassette encoding the globin protein is integrated into the genome of the cell.

4. (canceled)

5. The recombinant cell of claim 1, comprising at least two introduced expression cassettes encoding the globin protein, wherein the cassettes are integrated into the genome of the cell.

6. (canceled)

7. The recombinant cell of claim 1, wherein the expressed globin protein is selected from the group consisting of leghemoglobins, myoglobins, hemoglobins, cyanoglobins and non-symbiotic hemoglobins.

8. The recombinant cell of claim 1, wherein the expressed globin protein comprises a globin superfamily domain and a functional porphyrin (heme) binding site.

9. The recombinant cell claim 1, fermented for at least about 96 hours to about 300 hours, wherein the cell produces at least 0.1 grams of globin protein per liter of fermentation broth (g/L).

10. The recombinant cell claim 1, fermented for about 180 hours to about 190 hours, wherein the cell produces at least 1 grams of globin protein per liter of fermentation broth (g/L).

11. A method for producing a heterologous globin protein in a filamentous fungal cell comprising:
introducing an expression cassette encoding a globin protein and an expression cassette encoding a protease inhibitor into the filamentous fungal cell, and
fermenting the modified cell under suitable conditions for the production of the globin protein,
wherein the cell secretes the globin protein into the fermentation broth.

12. The method of claim 11, wherein the cassette encoding the globin protein is integrated into the genome of the cell.

13. The method of claim 11, wherein the cell comprises at least two introduced expression cassettes encoding the globin protein.

14. (canceled)

15. The method of claim 11, wherein the cell is fermented for at least about 96 hours to about 300 hours and produces at least 0.1 grams of globin protein per liter of fermentation broth (g/L).

16. The method of claim 11, wherein the cell is fermented for about 180 hours to about 190 hours and the cell produces at least 1 grams of globin protein per liter of fermentation broth (g/L).

17. The method of claim 11, wherein fermenting the modified cell under suitable conditions for the production of the globin protein does not require or include hemin media supplementation and/or does not or include require 5-aminolevulonic acid (ALA) media supplementation.

18. The method of claim 11, wherein the secreted globin is recovered from the fermentation broth, optionally wherein the recovered globin is purified.

19. The method of claim 11, wherein the globin protein is selected from the group consisting of leghemoglobins, myoglobins, hemoglobins, cyanoglobins and non-symbiotic hemoglobins.

20. The method of claim 19, wherein the leghemoglobin protein comprises at least 80% identity to the native soybean leghemoglobin protein of SEQ ID NO: 1 or at least 80% identity to the native French bean leghemoglobin protein of SEQ ID NO: 19.

21. The method of claim 19, wherein the myoglobin protein comprises at least 80% identity to the native bovine myoglobin protein of SEQ ID NO: 3.

22. The method of claim 19, wherein the globin protein comprises a globin superfamily domain and a functional porphyrin (heme) binding site.
